SEQUENCE LISTING (163 sequences)

SEQ ID NO: 1 - RNA, 14140 nt, Homo sapiens
  source 1..14140: mol_type = mRNA
gcactgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaacgatgccgcccgccgcgcccgcccgcctggcgctggccctgggcctgggcctgtggctcggggcgctggcggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagcctcgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccgggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagagcctcgacacccccccaacccccgcaccaagcagacaaagtcaataaaagagctgtctgactgcaa

SEQ ID NO: 2 - RNA, 209 nt, Homo sapiens
  source 1..209: mol_type = mRNA
gcactgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaacg

SEQ ID NO: 3 - RNA, 24 nt, Homo sapiens
  source 1..24: mol_type = mRNA
atgccgtccgcgggccccgcctga

SEQ ID NO: 4 - RNA, 18 nt, Homo sapiens
  source 1..18: mol_type = mRNA
atgcgcgcgctgccctaa

SEQ ID NO: 5 - RNA, 159 nt, Homo sapiens
  source 1..159: mol_type = mRNA
ctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaa

SEQ ID NO: 6 - RNA, 204 nt, Homo sapiens
  source 1..204: mol_type = mRNA
ctgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaa

SEQ ID NO: 7 - RNA, 14139 nt, Mus musculus
  source 1..14139: mol_type = mRNA
tccagtcccacctacagccaacttggaagcgcaggggcggagtctgagggcgccccgccccacgccggaggtgggggaggaggaggagctggcgctgggtctgcagtgcagcgcgaatgcgcgagcaggcggccaaggccctgaggtgcggcctgccccagagcgctgagcagctgtcgcaccgcagacgggccacggccgccgcggcattcagccccgtgccgcaccatgaggtccgcgggcccctcctgaactgcggctgccggcgcaaggggcctcggggaaccggggccatgcgcgggctgcgctgacgatgccgctcggcgcgcctgctctcctggcgctggccctaggcctgggcctgtggctcggagcgctggccggagaccctgggcgcggctgcgggccttgcccgctcccctgcttttgcggccctgcgcccgacgccgcctgccgcgtcaattgctccggccgctggctgcagacgctagggccgagtctgcgcatcccggctgacgccaccgcgctagacctgtcccacaacctacttcagacgctggacatagggctcctggtgaacctttcagcactggtggagctggacttaagcaacaataggatttctacattagaagaaggcgtgtttgctaatttatttaatttaagtgaaataaacctgagtgggaacccatttgagtgcaactgtggtctggcgtggctgccacgctgggcaaaggaacatcaggtgcatgtggtacagtctgaggcaaccacgtgcagggggcccattccactggccggccaacctctcctcagtatccccttattggacaatgcctgtggtgaggaatatgtcgcctgcctccctgacaacagctcaggtgctgtggcagcagtacccttctactttgcccatgagggaccactggagactgaggcctgcagtgccttctgcttctcagctggtgagggccttgcagccctctcggagcagaatcaatgcctctgtggggcaggccaggcctctaactcttctgcagcctgctcatcctggtgttccagcatctcactgtccctcaactctgcctgtgggggccccaccttgctccagcacacctttcccgcctccccaggggctaccctggtggggccccatggacccctggcctctggccagccagcagacttccatattacttcctccctgcccattagctcaacacgctggaactttggagatggctcacctgaggtggacatggctagccctgctgccacccacttctatgtgctacctgggagctatcacatgacagttgtgctggccttgggggctggctcagctctgctggagacagaggtgcaagtagaagcaacacctactgtcctggagcttgtgtgcccatcctttgtgcacagtaacgagagccttgaacttggcatacggcatcgtggaggctcagcccttgaggtcacctatagcatcctggctttggataaagagcctgcccaagtggtccacccactctgccccttggacacagagatcttccctggcaatgggcactgctaccgcctggtggcagagaaggcaccctggctgcaagcacaggagcaatgtcggacttgggctggagctgccttggctatggtggacagtcctgccatccagcacttcctagtctccaaggttaccaggagcctggatgtgtggatcggcttctcatctgtggaggggacagaaggcctggatccccggggtgaggccttcagcctggaaagctgccagaactggctgcctggggagccacacccagccacagcagagcattgtgtgcggttggggcctgctgggcaatgcaacacagacctgtgttcagcaccacatagttatgtctgcgagctgcggcctggagggcctgtgtgggatactgagaactttgtcatgggaatgtctggtggaggcctgtctggacctctgcatccattggcacagcaggaaactgtccagggcccgctgcgacctgtggaggtcatggtgttccctggcctgagccctagccgggaagcctttctcactgcagcagagttttctacccagaagcttgaagagcctgcccagatgcggctgcaggtgtaccggccctcaggaggagcagcagcggtcccagaaggcagcagtgagcctgacaacaggactgagccggcccccaagtgtgtgcctgaggaactctggtgtccaggagccaatgtctgcataccttttgatgcttcgtgtaactctcatgtctgcatcaatgggtctgtgtcaaggctgggactctctagggccagctacacactatggaaggagttctttttttctgtgcctgcagggcctcccacacagtacttggtcaccctccacagccaggatgttcctatgctccctggtgacctcattggcttacagcatgatgctggcccaggtactctcctacagtgtcccctggcttccagctgtcctggccaagccctgtacctttcaaccaatgcctcagattggatgaccaacctgcctgtccatctggaagaagcttgggctggccctgtctgctccctacagctgcttttggtcacagagcggcttacaccactgctgggccttgggcctaaccctggactacagcatcctgggcactatgaggtccgggctacagtgggcaacagcgtatccaggcaaaacctgtcctgcagcttcagtgtggtctctccaatagctgggctgagagtcattcaccccatcccccttgatggccacatctatgtgccaaccaacggctcagtcttggtgcttcaggtagattctggtgctaatgccactgccacagctcagtggtttgggggcaatattagtgctcccttcgaggatgcctgccctcctgaggtggattttctcaagcaagattgcacagaagaggccaatggcaccctgttctcagtgttgatgttgcccaggctcaaggagggggatcacacagtggagattgtggcgcaaaatggagccagccaggccaacctcagcctgagagtgacagctgaggagcccatctgtggcctccgtgctgtgccaagccctgaggcccgagtgctacagggcatcctggtgcggtacagccccatggtagaggctggctcagacgtagccttccggtggaccattgatgataagcagtctctgactttccacaacactgtcttcaatgtaatataccagagtgctgccatcttcaagctctcgctgacagcctctaaccatgtgagcaacatcaccgtgaactacaatgtcactgtggagcggatgaacaagatgcatgggctgtgggtatctgcagtaccgactgtgttaccccccaatgccacactggcactgacaggtggtgtgctggtggattcagctgtggaggtggccttcctgtggaactttggggatggggagcaggtactccgccagttcaagcctccatacgacgagtccttccaggttccagaccccacagtggcgcaggtgctggtggaacacaataccacgcatatttacactaccccaggtgagtacaacttgaccgtccttgtgtccaatacctatgagaatctgacgcagcaggtgacagtaagtgtgcgcactgttcttcccaacgtggctattggcatgagcagcaacgtcctggtggccggccaacccatcaccttttccccatatccgctgccctcaactgatggtgtcctatatacgtgggactttggggatggctcacctgtcctaatacagagccagccagtactcaaccacacctacagcatgacgggcgcttacagaatcaccttggaggttaacaacacagtgagcagcgtgacagcacatgcagacatccgtgtcttccaggagttgcatggcctgactgtgtatttgagcccatctgtggagcagggagcccccatggtagtcagtgcttctgtggagtcaggtgacaacattacatggacatttgacatgggagatggcacagtattcacaggccctgaggctacagtacaacatgtatacctgcgggcccagaacttcacagtgacagtagaagcagccaaccctgctggccacttgtcccagagcctgcatgtgcaagtgtttgtcctggaggtattacacatagagccctctacctgcatccccacacagcccagtgcccagcttatggcccatgtcactggggaccctgtccattacctctttgattggacttttggagatggctcctccaatgtgactgtccatgggcatccatcagtgacacacaacttcactaggagtggcatattcccactggcattggttttgtcaagccatgtgaacaaggcacattattttaccagcatctgtgtggaacctgagatacgtaacatcaccctgcagcctgagaggcagtttgtgaagctgggggatgaagcccggctggttgcctattcttggcccccattcccgtaccgatatacctgggactttggcactgaagacactacccacacccaaactgggggctctgaagtgaaatttatctaccgagaaccaggctcctacttggtgatagtcactgtgtccaacaacatctcttctaccaatgactcagcttttgtggaggtccaggagcctgtgttagtcactggcatcaggatcaatggctcccatgtgttagagctgcagcagccttacctgttgtcagccatgggcagtgggagccctgccacctacctatgggaactgggagacggcagccagagtgagggccctgaggtcacccatatctacagcagcaccggtgatttcactgtcagggtgtctggctggaatgaggtgagccgtagtgaggcccaactcaacatcaccgtaaagcagcgtgtgcggggcctcaccatcaatgccagccgcacagtggtgcccctgaatggcagtgtgagctttagtaccttgttggaggtagggagtgatgtgcattactcctgggtgctctgtgacagatgcacacccattccaggcggacccactatttcctacaccttccgctctgtgggcaccttcaatattattgtcacagctgagaatgaggtgggttctgcccaggacagcatcttcatctatgttctccagttcattgaggggctgcaggtggcagggggtgataacggctgctgcttccctaccaactacacactgcagctgcaagctgcagttagggatggcaccaacatctcttatagctggacagcgcagcaggagggcagcctcatcacactcttcggcagtggcaagtgcttttctctcacttcactcaaagctagcacctactatgtccatcttagggccactaacatgttgggtagtgccgcagccaaccgtactatagactttgtggaacctgtggagagtctaatcctatctgcatcccctaatccagctgctgtcaacatgagtctcaccctttgtgctgaattggctggtggcagtggtgttgtttacacttggtacctggaggaaggactgagttggaagacctccatgccatctaccacccacacctttgctgcacctggcctgcacttggtcagagtcacagctgagaaccagttgggctcagtcaatgccacagttgaagtggccatacaggtgcctgtgggtggtctgagtatcagaaccagtgagccagatagcatctttgtggcagctggctccactctacccttctggggtcagctggctgaaggtaccaatgtgacttggtgctggaccttgccaggtgggagtaaggacagccagtacattgctgtgcgcttctccacggctggcagtttctctctgcagctcaatgcttccaatgcagtcagttgggtctcagccatgtacaacctcacagtagaggaacccattgtgaacctgatgctgtgggccagcagcaaggtggtggcgcctgggcagccagtccactttgaaatcctgctggcagctggctctgctcttactttccgacttcaggttggtgggtctgtccctgaagtgctccctagcccccacttctcccacagcttctttcgggttggagaccatctggtgaatgtgcaggctgagaaccacgtgagtcatgcccaggcacaggttcgcatcctagtgctggaggctgttgttgggctgcaggtacccaactgctgtgagccaggcatggccacaggcactgagaagaacttcacagcccgggtgcagcgaggttcccgagttgcctatgcctggtatttctccttgcaaaaggttcagggtgactcattggttattttatctggccgtgatgtcacctacacacctgtggctgcagggttactggagatccatgtgcgtgcctttaatgaactgggtggtgtgaacctcacactcatggtggaagtccaggatatcatccagtatgtaacacttcagagtggccgctgctttaccaaccgctcagccaggtttgaggctgctacaagccccagtccacggcgtgtaacctaccactgggactttggggatgggactccggtacagaaaacagaggaattctgggctgatcactactacctgcgtcctggggactaccatgtagaggtgaatgctacaaacctagtgagtttttttgtggcacaggccacagtgactgtccaggtgctggcctgtagggagcctgaagtggaggtagcactgcctctgcaggtgctgatgcggcgttcccagcgcaactacctggaggcccatgttgatctacgcaactgtgtctcctaccagactgaatatcggtgggagatatatcgtactgccagctgccaacggccaggacgtatggctcaaatggtactgcctggtgtggatgtgagccgaccccagctggtggtgccacggctggcactacctgtgggccactactgctttgtgtttgtggtttcatttggggacacaccattggcacggagcatccaggccaatgtgacagtggctgctgagcgtctggtacccatcattgaaggtggctcataccgggtatggtcagacacgcaggacctggtgctggatgggagcaagtcctatgaccctaatttagaggatggtgatcagacaccgctcaacttccattgggcctgcgtggcttcaacacagagtgagacaggtggctgcgtgctaaactttgggccccgtgggagcagtgtggtcaccatccccctggaacgcctagaagctggtgtagaatataccttcaacctcatagtgtggaaagcaggtcggaaggaagaagccaccaaccagacagtactgatccgaagtgggcgagtccccattgtgtcccttgagtgtgtgtcctgcaaggcacaagcagtgtatgaagtgagccgaagttcctatgtgtacttggagggtcactgccacaactgcagcagaggctacaagcaagggtgttgggccgctcgcactttcagcaataagactctggtgctgaacgagacgacaacatccacgggtagcaccggcatgaatctggtagtgcggccaggtgcactgcgtgatggggagggctacatcttcacactcactgtgctgggccactcaggggaagaagagggctgtgcctcaatccgcctctcacccaaccgccccccacttggaggctcctgccgcctattcccattggattctgtgcgtggcctcaccactaaggttcacttcgagtgcacaggttggcgtgatgcagaagatggtggagccccactggtatatgctctgttgctgaagcgctgtcgtcagagctactgtgagaatttctgcatctataagggtagtctttccacctatggggctgtcctgcctcctggattccagcccctttttgtggtgagcctagctgtggttgtacaagaccagctgggtgctgctgttgttgcactcaacaggtctctgaccattgtcctaccagaacccagtggaaatcctgcagacctcgtaccctggctgcacagcctgactgccagtgtgctgcctgggctcctgaagcaggctgaccctcagcatgtcatcgagtactctttagccctcattactgtgctcaatgagtatgagcaggccccagatgtgtcagagcccaacgttgagcagcagctgagagcccaaatgcgtaagaacatcacggagaccctgatctccctgcgggtcaacactgtggatgacatccagcagattactgctgcgttagcccagtgcatggtgtccagcagggagctcatgtgccgctcctgcctgaagaagatgctgcagaagctggagggcatgatgcgcatcttgcaagctgaaaccactgagggcaccttgacacccaccaccattgctgacagcattctcaatatcacaggtgacctcatccacctggccagcttggacatgcagggcccacagcccttggagctgggggtggagccgccctccctgatggtggcttctaaggcctacaacctctcgtcggccctcatgcgtatcctcatgcgttcccgagtgctcaatgaggagcctctgactctggctggcgaggagattgtggccctaggcaagcgctcagacccactcagtcttctgtgttatggcaaagccttggggcccagctgccacttctccattcctgaggccttcagtggggccctgtcgaacctcagtgatgtggtgcagctcatcttccttgtggattccaaccccttcccctttggctacattagcaactacactgtctccaccaaagtggcatctatggcattccagacacagactggtacccagatccctattgagcagttggctgcagagcgtgccatcacagtgaaggtgccaaacaactcagaccaggctgcccagagctctcacaatcctgtgggttctaccattgtacagccccagacctctgtcagcgctgtggtcactgcagacaacagtaaccctcaagctggcctgcatctgaggatcacttacacagtgcttaatgagcgctatctgtccgcggagcctgagccctacctggctgtgtacctgcactcagtgtcccagcccaatgagtacaactgctcggccagcaggaggatcagcctggaggtgctggagggtgctgaccacaggctctacaccttcttcattgccccaggaactgggaccttggacaggagttactacctgaacctgacgagccatttccactggtctgccctggaggtgtctgtgggcctctatacatccctgtgccagtacttcagtgaggagatgatgatgtggagaactgagggcattgtacccctggaggagacctcacccagccaggctgtctgcctcacccgccacctcactgccttcggtgccagcctttttgtgcctcccagccatgtccagttcatctttcctgaaccatctgcaagcatcaactacattgtcctcctgacgtgtgtcatatgcctggtgacctatgtggtcatggctatgatcctgcgcaagctggaccagctggatgttagccgtgtccgtgtcatcccgttctgtgggaaggggggccgcttcaaatatgaaatacttgttaagaccggctggagccgaggttcaggtaccactgctcacgtcggcatcatgctgtatggagaggacaaccgtagtggtcaccggcacttagatggggacagagcctttcatcgaaacagcctggacatcttccaaattgccaccccgcacagcctgggcagcgtgtggaagatccgagtgtggcatgataacaaagggctcagccctgcctggttcctgcaacacattatcgtccgggacctacagagtgcccgcagcaccttctttttggtcaatgactggctgtctgtggagactgaggccaacgggggcctagtggagaaggaggtgcttgcagcaaatgaagctgcgctgtggcagttccagcggctcctcgtggctgagttgcagagaggcttctttgacaagcacatctggctctccatatgggaccggccgcctcgtagccgctttactagagtccagagggttacctgctgtgttctcctcctctgcctcttcctggctgccaatgctgtgtggtacggagttgtgagagacaccacctacagcatgggacctgtgtccagtctgatctctcctggtgttgacacagttgctattggccttgtgtccagtgtggtagtctaccctgtctatttggctgtcctgttcctcttccggatgtctcggagtaaggtgtctggggaccaaaaccccacacccacagggcagcaggccctagatgttgacagctacctggacccatctgtgctagacagctccctcctcacactctctggcctcacagaggccttcgctgggcaagtgaagaatgacttgtttctggaagatgctaaaagcctggtgtgctggccgtccagtgagggtactcttagttggccagacctgctcagtgacccatctgttgtgagcagcacactgcagcgattgacccagggcaggcctggctgcatgttgggctcagaggaagatggtgcctctctggttagcccttccttgcctgccaaatacttgtcggcctcagatgaagacctgatccatcaggtactggctgatggggccaacaacctggttcctacccaggacaccctcctagaaacagacctgcttactagcctgtccagtgttcctggagagaaaacagaaactctgatactgcagacagtgggggaggagagaccagccagcatgggcctcagttgggaacagtccccggtgacaaggctttccaggacaggactggtggaaggttttcagaagcgactgctgcctgcctggtgtgccccactggcccatgggctcagcctgctcttggtagctgtagctgtagcagtttcagggtggattggtgccagcttccctcccagcgtgagtgtcatgtggcttctctcaagcagctccagcttcctggcctcctttcttggctgggagccccttaaggtcctgctggaagccctctacttctcccttgtggccaagcggcttcaccctgatgaagatgacactctggtagagagcccagctgtaacccctgtaagtgagcgtgtgccccgtgtgcggcccccacatggttttgcactcttcttggcaaaggaggaagctcgaaaggtcaagaggctccatgacatgttgaagagactcttggtgtacatgcttttcttactggtgacgctgctggccaactatggggatgcttcttgccatggtcacgcctatcgcctgcagagcgccatcaagcaggagctggatagccaagcctttctggccatcacccggtctgatgagttctggccatggatgtcacatgtgttcttgccctatgtccatgggaaccagtctagccctgagctgggccctccacggctacggcaggtgcgactgcaggaagcattctgcccagacccttctagctcggagcacatgtgttcagcggcaggaagcttgagcaccagtgactatggcattggctggcagagtgtggtgcaaaatggctctgagacatgggcctactctgcaccggacctgctgggcgcctggtactggggctactgtgcagtgtatgacagtgggggctacatacaggagttaggactgagtctggaggagagccgtgcacgactgggcttcctgcagctgcacaactggcttgacagcaggagccgtgcagtgtttgtagaactcacccgctacagccctgctgtggggctccatgctgctgttacactgcgcctcgagttccctgtagccggccatgcactggctgccttcagtgtccgcccatttgcgttacggcgactcagcactggcctgtctctaccactgctcacctcggtgtgcttgctgctgtttgccctatacttttccatggccgaggtgcagacctggcgcaaagatgggtgtgcgtgcacagcaaggcctgacacctgggcacggtgcctactggtgatactgacagcagctacagggcttgtacgcctggcccagctgggcattgctgatcgccagtggacccattttgtgcaggaccatccacgccacttcactagcttcgaccaagtggctcagctgggctctgtggcccgtggcctggcagcttcactactcttcctgcttttggtcaaggctgcccagcagctgcggtttgtgcgccagtggtctgtttttggcaagacattgtgtcgggccttgcccgagctcatgggagccaccttgggcctggtgctgcttggtgtggcctatgcacagatggccatcctgcttatttcctctggtgctgacactctctacaacatggcccgtgccttcctggtgctgtgccctggggcgagggtccccactctgtgcccttcagagtcctggtacctgtcccctctgctgtgtgtggggctctgggctctgcgagtgtggggtgccctgaggttaggggctatcctcctgcgttggcggtaccacgccttgcgtggcgagctctaccgcccagcatgggaaccccaggactatgagatggtggagcttttcctgcgtaggcttcggctctggatgggcttcagcaaggtcaaggagttccgccacaaagtccgctttgaaggaatggatccactgccttcccgctcatccaggggctccaagtcatccccagttgtgctcccacctagctcaggctcagaagcttcacacccatccacctcgtccagccaaccagacgggccgagcgccagcttaagccgctcgacgctgaagctggaaccagagccctctcgcctccatgctgtgtttgaaagtctgcttgtccagtttgaccgactcaaccaggccacagaggacgtctaccagctggagcaacaactccagagccttcaaggccatgggcacaatggacctccttcctctccctcccctggctgcttcccaggctctcagccagctttgcccagccgcctttctcgggccagtcaggggctggatcagactgtaggccccaacagggtgtccctgtggcctaataacaaggtccaccccagcagcacttaggccctaggggtcttggccattcccttccctgggaatgcctgagcttcacactggcctctcagagccagggtggacaccactcagtattaccttctgctgccctctaggttgggccaggcagaacggctgcatgccagttcttttgggtacaggtattgctgccttccttacctgtccacatatggggcttctgcactttaaaaaggctgtgtggccagccaggacccagggtcccctccccacaggaggacacagcagtattggaccaagtgagtacccagcctccaagatgctaatttattcgccccccctccccccaagtcctcaggttcagcgggctgcgcccagctctacccctaggtggccatctcctcttgctaaaaatctaagctggagggagggctaagctacacttctcctttatccgtcccctaagttattacctctcttgtttctgcagcatacttgccctctagctaccatctgctttctatgtccaccatcaataatttatatggggttaaaatgtatatatttttgtatgtcagtattttctacttgggctgaaaatgggtccattgttacccagggcaggggtacaggggaagctgcagctattactggatccgagtctggcctcgggcaggtgctggacagcaattggggccaccgtggccttgttttcctcccttgtctggggccaggcagcacgcagatctgctggtttcagatctgagcaagggcaggactactgtgggatgaaagatgccaagaagttactggacaagcccaccaatggggcttgcccccatggcgggtgggggaaagagaatgtatgtgatggcacctgcctgcttctgtgggctcatctgcagctctagcctggattctgccccaaccccaaacagacaagacaaagtcaaataaagaagctgtctgactgc

SEQ ID NO: 8 - RNA, 313 nt, Mus musculus
  source 1..313: mol_type = mRNA
tccagtcccacctacagccaacttggaagcgcaggggcggagtctgagggcgccccgccccacgccggaggtgggggaggaggaggagctggcgctgggtctgcagtgcagcgcgaatgcgcgagcaggcggccaaggccctgaggtgcggcctgccccagagcgctgagcagctgtcgcaccgcagacgggccacggccgccgcggcattcagccccgtgccgcaccatgaggtccgcgggcccctcctgaactgcggctgccggcgcaaggggcctcggggaaccggggccatgcgcgggctgcgctgacg

SEQ ID NO: 9 - RNA, 24 nt, Mus musculus
  source 1..24: mol_type = mRNA
atgaggtccgcgggcccctcctga

SEQ ID NO: 10 - RNA, 18 nt, Mus musculus
  source 1..18: mol_type = mRNA
atgcgcgggctgcgctga

SEQ ID NO: 11 - protein, 30 aa, Homo sapiens
MPPAAPARLALALGLGLWLGALAGGPGRGC

SEQ ID NO: 12 - protein, 16 aa, Mus musculus
MPLGAPALLALALGLG

SEQ ID NO: 13 - RNA, 37 nt, Homo sapiens
  source 1..37: mol_type = mRNA
ccagccccgcgcccgccatgccgtccgcgggccccgc

SEQ ID NO: 14 - RNA, 20 nt, Homo sapiens
  source 1..20: mol_type = mRNA
ccagccccgcgcccgccatg

SEQ ID NO: 15 - RNA, 20 nt, Homo sapiens
  source 1..20: mol_type = mRNA
cagccccgcgcccgccatgc

SEQ ID NO: 16 - RNA, 20 nt, Homo sapiens
  source 1..20: mol_type = mRNA
agccccgcgcccgccatgcc

SEQ ID NO: 17 - RNA, 20 nt, Homo sapiens
  source 1..20: mol_type = mRNA
gccccgcgcccgccatgccg

SEQ ID NO: 18 - RNA, 20 nt, Homo sapiens
  source 1..20: mol_type = mRNA
ccccgcgcccgccatgccgt

SEQ ID NO: 19 - RNA, 20 nt, Homo sapiens
  source 1..20: mol_type = mRNA
cccgcgcccgccatgccgtc

SEQ ID NO: 20 - RNA, 20 nt, Homo sapiens
  source 1..20: mol_type = mRNA
ccgcgcccgccatgccgtcc

SEQ ID NO: 21 - RNA, 20 nt, Homo sapiens
  source 1..20: mol_type = mRNA
cgcgcccgccatgccgtccg

SEQ ID NO: 22 - RNA, 20 nt, Homo sapiens
  source 1..20: mol_type = mRNA
gcgcccgccatgccgtccgc

SEQ ID NO: 23 - RNA, 20 nt, Homo sapiens
  source 1..20: mol_type = mRNA
cgcccgccatgccgtccgcg

SEQ ID NO: 24 - RNA, 20 nt, Homo sapiens
  source 1..20: mol_type = mRNA
gcccgccatgccgtccgcgg

SEQ ID NO: 25 - RNA, 20 nt, Homo sapiens
  source 1..20: mol_type = mRNA
cccgccatgccgtccgcggg

SEQ ID NO: 26 - RNA, 20 nt, Homo sapiens
  source 1..20: mol_type = mRNA
ccgccatgccgtccgcgggc

SEQ ID NO: 27 - RNA, 20 nt, Homo sapiens
  source 1..20: mol_type = mRNA
cgccatgccgtccgcgggcc

SEQ ID NO: 28 - RNA, 20 nt, Homo sapiens
  source 1..20: mol_type = mRNA
gccatgccgtccgcgggccc

SEQ ID NO: 29 - RNA, 20 nt, Homo sapiens
  source 1..20: mol_type = mRNA
ccatgccgtccgcgggcccc

SEQ ID NO: 30 - RNA, 20 nt, Homo sapiens
  source 1..20: mol_type = mRNA
catgccgtccgcgggccccg

SEQ ID NO: 31 - RNA, 20 nt, Homo sapiens
  source 1..20: mol_type = mRNA
atgccgtccgcgggccccgc

SEQ ID NO: 32 - RNA, 18 nt, Homo sapiens
  source 1..18: mol_type = mRNA
agccccgcgcccgccatg

SEQ ID NO: 33 - RNA, 18 nt, Homo sapiens
  source 1..18: mol_type = mRNA
gccccgcgcccgccatgc

SEQ ID NO: 34 - RNA, 18 nt, Homo sapiens
  source 1..18: mol_type = mRNA
ccccgcgcccgccatgcc

SEQ ID NO: 35 - RNA, 18 nt, Homo sapiens
  source 1..18: mol_type = mRNA
cccgcgcccgccatgccg

SEQ ID NO: 36 - RNA, 18 nt, Homo sapiens
  source 1..18: mol_type = mRNA
ccgcgcccgccatgccgt

SEQ ID NO: 37 - RNA, 18 nt, Homo sapiens
  source 1..18: mol_type = mRNA
cgcgcccgccatgccgtc

SEQ ID NO: 38 - RNA, 18 nt, Homo sapiens
  source 1..18: mol_type = mRNA
gcgcccgccatgccgtcc

SEQ ID NO: 39 - RNA, 18 nt, Homo sapiens
  source 1..18: mol_type = mRNA
cgcccgccatgccgtccg

SEQ ID NO: 40 - RNA, 18 nt, Homo sapiens
  source 1..18: mol_type = mRNA
gcccgccatgccgtccgc

SEQ ID NO: 41 - RNA, 18 nt, Homo sapiens
  source 1..18: mol_type = mRNA
cccgccatgccgtccgcg

SEQ ID NO: 42 - RNA, 18 nt, Homo sapiens
  source 1..18: mol_type = mRNA
ccgccatgccgtccgcgg

SEQ ID NO: 43 - RNA, 18 nt, Homo sapiens
  source 1..18: mol_type = mRNA
cgccatgccgtccgcggg

SEQ ID NO: 44 - RNA, 18 nt, Homo sapiens
  source 1..18: mol_type = mRNA
gccatgccgtccgcgggc

SEQ ID NO: 45 - RNA, 18 nt, Homo sapiens
  source 1..18: mol_type = mRNA
ccatgccgtccgcgggcc

SEQ ID NO: 46 - RNA, 18 nt, Homo sapiens
  source 1..18: mol_type = mRNA
catgccgtccgcgggccc

SEQ ID NO: 47 - RNA, 18 nt, Homo sapiens
  source 1..18: mol_type = mRNA
atgccgtccgcgggcccc

SEQ ID NO: 48 - RNA, 16 nt, Homo sapiens
  source 1..16: mol_type = mRNA
ccccgcgcccgccatg

SEQ ID NO: 49 - RNA, 16 nt, Homo sapiens
  source 1..16: mol_type = mRNA
cccgcgcccgccatgc

SEQ ID NO: 50 - RNA, 16 nt, Homo sapiens
  source 1..16: mol_type = mRNA
ccgcgcccgccatgcc

SEQ ID NO: 51 - RNA, 16 nt, Homo sapiens
  source 1..16: mol_type = mRNA
cgcgcccgccatgccg

SEQ ID NO: 52 - RNA, 16 nt, Homo sapiens
  source 1..16: mol_type = mRNA
gcgcccgccatgccgt

SEQ ID NO: 53 - RNA, 16 nt, Homo sapiens
  source 1..16: mol_type = mRNA
cgcccgccatgccgtc

SEQ ID NO: 54 - RNA, 16 nt, Homo sapiens
  source 1..16: mol_type = mRNA
gcccgccatgccgtcc

SEQ ID NO: 55 - RNA, 16 nt, Homo sapiens
  source 1..16: mol_type = mRNA
cccgccatgccgtccg

SEQ ID NO: 56 - RNA, 16 nt, Homo sapiens
  source 1..16: mol_type = mRNA
ccgccatgccgtccgc

SEQ ID NO: 57 - RNA, 16 nt, Homo sapiens
  source 1..16: mol_type = mRNA
cgccatgccgtccgcg

SEQ ID NO: 58 - RNA, 16 nt, Homo sapiens
  source 1..16: mol_type = mRNA
gccatgccgtccgcgg

SEQ ID NO: 59 - RNA, 16 nt, Homo sapiens
  source 1..16: mol_type = mRNA
ccatgccgtccgcggg

SEQ ID NO: 60 - RNA, 16 nt, Homo sapiens
  source 1..16: mol_type = mRNA
catgccgtccgcgggc

SEQ ID NO: 61 - RNA, 16 nt, Homo sapiens
  source 1..16: mol_type = mRNA
atgccgtccgcgggcc

SEQ ID NO: 62 - RNA, 20 nt, Homo sapiens
  source 1..20: mol_type = mRNA
ctggggacggcggggccatg

SEQ ID NO: 63 - RNA, 20 nt, Homo sapiens
  source 1..20: mol_type = mRNA
tggggacggcggggccatgc

SEQ ID NO: 64 - RNA, 20 nt, Homo sapiens
  source 1..20: mol_type = mRNA
ggggacggcggggccatgcg

SEQ ID NO: 65 - RNA, 20 nt, Homo sapiens
  source 1..20: mol_type = mRNA
gggacggcggggccatgcgc

SEQ ID NO: 66 - RNA, 20 nt, Homo sapiens
  source 1..20: mol_type = mRNA
ggacggcggggccatgcgcg

SEQ ID NO: 67 - RNA, 20 nt, Homo sapiens
  source 1..20: mol_type = mRNA
gacggcggggccatgcgcgc

SEQ ID NO: 68 - RNA, 20 nt, Homo sapiens
  source 1..20: mol_type = mRNA
acggcggggccatgcgcgcg

SEQ ID NO: 69 - RNA, 20 nt, Homo sapiens
  source 1..20: mol_type = mRNA
cggcggggccatgcgcgcgc

SEQ ID NO: 70 - RNA, 20 nt, Homo sapiens
  source 1..20: mol_type = mRNA
ggcggggccatgcgcgcgct

SEQ ID NO: 71 - RNA, 20 nt, Homo sapiens
  source 1..20: mol_type = mRNA
gcggggccatgcgcgcgctg

SEQ ID NO: 72 - RNA, 20 nt, Homo sapiens
  source 1..20: mol_type = mRNA
cggggccatgcgcgcgctgc

SEQ ID NO: 73 - RNA, 20 nt, Homo sapiens
  source 1..20: mol_type = mRNA
ggggccatgcgcgcgctgcc

SEQ ID NO: 74 - RNA, 20 nt, Homo sapiens
  source 1..20: mol_type = mRNA
gggccatgcgcgcgctgccc

SEQ ID NO: 75 - RNA, 20 nt, Homo sapiens
  source 1..20: mol_type = mRNA
ggccatgcgcgcgctgccct

SEQ ID NO: 76 - RNA, 20 nt, Homo sapiens
  source 1..20: mol_type = mRNA
gccatgcgcgcgctgcccta

SEQ ID NO: 77 - RNA, 20 nt, Homo sapiens
  source 1..20: mol_type = mRNA
ccatgcgcgcgctgccctaa

SEQ ID NO: 78 - RNA, 20 nt, Homo sapiens
  source 1..20: mol_type = mRNA
catgcgcgcgctgccctaac

SEQ ID NO: 79 - RNA, 20 nt, Homo sapiens
  source 1..20: mol_type = mRNA
atgcgcgcgctgccctaacg

SEQ ID NO: 80 - RNA, 18 nt, Homo sapiens
  source 1..18: mol_type = mRNA
ggggacggcggggccatg

SEQ ID NO: 81 - RNA, 18 nt, Homo sapiens
  source 1..18: mol_type = mRNA
gggacggcggggccatgc

SEQ ID NO: 82 - RNA, 18 nt, Homo sapiens
  source 1..18: mol_type = mRNA
ggacggcggggccatgcg

SEQ ID NO: 83 - RNA, 18 nt, Homo sapiens
  source 1..18: mol_type = mRNA
gacggcggggccatgcgc

SEQ ID NO: 84 - RNA, 18 nt, Homo sapiens
  source 1..18: mol_type = mRNA
acggcggggccatgcgcg

SEQ ID NO: 85 - RNA, 18 nt, Homo sapiens
  source 1..18: mol_type = mRNA
cggcggggccatgcgcgc

SEQ ID NO: 86 - RNA, 18 nt, Homo sapiens
  source 1..18: mol_type = mRNA
ggcggggccatgcgcgcg

SEQ ID NO: 87 - RNA, 18 nt, Homo sapiens
  source 1..18: mol_type = mRNA
gcggggccatgcgcgcgc

SEQ ID NO: 88 - RNA, 18 nt, Homo sapiens
  source 1..18: mol_type = mRNA
cggggccatgcgcgcgct

SEQ ID NO: 89 - RNA, 18 nt, Homo sapiens
  source 1..18: mol_type = mRNA
ggggccatgcgcgcgctg

SEQ ID NO: 90 - RNA, 18 nt, Homo sapiens
  source 1..18: mol_type = mRNA
gggccatgcgcgcgctgc

SEQ ID NO: 91 - RNA, 18 nt, Homo sapiens
  source 1..18: mol_type = mRNA
ggccatgcgcgcgctgcc

SEQ ID NO: 92 - RNA, 18 nt, Homo sapiens
  source 1..18: mol_type = mRNA
gccatgcgcgcgctgccc

SEQ ID NO: 93 - RNA, 18 nt, Homo sapiens
  source 1..18: mol_type = mRNA
ccatgcgcgcgctgccct

SEQ ID NO: 94 - RNA, 18 nt, Homo sapiens
  source 1..18: mol_type = mRNA
catgcgcgcgctgcccta

SEQ ID NO: 95 - RNA, 18 nt, Homo sapiens
  source 1..18: mol_type = mRNA
atgcgcgcgctgccctaa

SEQ ID NO: 96 - RNA, 16 nt, Homo sapiens
  source 1..16: mol_type = mRNA
ggacggcggggccatg

SEQ ID NO: 97 - RNA, 16 nt, Homo sapiens
  source 1..16: mol_type = mRNA
gacggcggggccatgc

SEQ ID NO: 98 - RNA, 16 nt, Homo sapiens
  source 1..16: mol_type = mRNA
acggcggggccatgcg

SEQ ID NO: 99 - RNA, 16 nt, Homo sapiens
  source 1..16: mol_type = mRNA
cggcggggccatgcgc

SEQ ID NO: 100 - RNA, 16 nt, Homo sapiens
  source 1..16: mol_type = mRNA
ggcggggccatgcgcg

SEQ ID NO: 101 - RNA, 16 nt, Homo sapiens
  source 1..16: mol_type = mRNA
gcggggccatgcgcgc

SEQ ID NO: 102 - RNA, 16 nt, Homo sapiens
  source 1..16: mol_type = mRNA
cggggccatgcgcgcg

SEQ ID NO: 103 - RNA, 16 nt, Homo sapiens
  source 1..16: mol_type = mRNA
ggggccatgcgcgcgc

SEQ ID NO: 104 - RNA, 16 nt, Homo sapiens
  source 1..16: mol_type = mRNA
gggccatgcgcgcgct

SEQ ID NO: 105 - RNA, 16 nt, Homo sapiens
  source 1..16: mol_type = mRNA
ggccatgcgcgcgctg

SEQ ID NO: 106 - RNA, 16 nt, Homo sapiens
  source 1..16: mol_type = mRNA
gccatgcgcgcgctgc

SEQ ID NO: 107 - RNA, 16 nt, Homo sapiens
  source 1..16: mol_type = mRNA
ccatgcgcgcgctgcc

SEQ ID NO: 108 - RNA, 16 nt, Homo sapiens
  source 1..16: mol_type = mRNA
catgcgcgcgctgccc

SEQ ID NO: 109 - RNA, 16 nt, Homo sapiens
  source 1..16: mol_type = mRNA
atgcgcgcgctgccct

SEQ ID NO: 110 - RNA, 20 nt, Homo sapiens
  source 1..20: mol_type = mRNA
cgagctcccggagcggcctg

SEQ ID NO: 111 - RNA, 20 nt, Homo sapiens
  source 1..20: mol_type = mRNA
gagctcccggagcggcctgg

SEQ ID NO: 112 - RNA, 20 nt, Homo sapiens
  source 1..20: mol_type = mRNA
agctcccggagcggcctggc

SEQ ID NO: 113 - RNA, 20 nt, Homo sapiens
  source 1..20: mol_type = mRNA
gctcccggagcggcctggcc

SEQ ID NO: 114 - RNA, 20 nt, Homo sapiens
  source 1..20: mol_type = mRNA
ctcccggagcggcctggccc

SEQ ID NO: 115 - RNA, 20 nt, Homo sapiens
  source 1..20: mol_type = mRNA
tcccggagcggcctggcccc

SEQ ID NO: 116 - RNA, 20 nt, Homo sapiens
  source 1..20: mol_type = mRNA
cccggagcggcctggccccg

SEQ ID NO: 117 - RNA, 20 nt, Homo sapiens
  source 1..20: mol_type = mRNA
ccggagcggcctggccccga

SEQ ID NO: 118 - RNA, 20 nt, Homo sapiens
  source 1..20: mol_type = mRNA
cggagcggcctggccccgag

SEQ ID NO: 119 - RNA, 20 nt, Homo sapiens
  source 1..20: mol_type = mRNA
ggagcggcctggccccgagc

SEQ ID NO: 120 - RNA, 20 nt, Homo sapiens
  source 1..20: mol_type = mRNA
gagcggcctggccccgagcc

SEQ ID NO: 121 - RNA, 20 nt, Homo sapiens
  source 1..20: mol_type = mRNA
agcggcctggccccgagccc

SEQ ID NO: 122 - RNA, 20 nt, Homo sapiens
  source 1..20: mol_type = mRNA
gcggcctggccccgagcccc

SEQ ID NO: 123 - RNA, 20 nt, Homo sapiens
  source 1..20: mol_type = mRNA
cggcctggccccgagccccg

SEQ ID NO: 124 - RNA, 20 nt, Homo sapiens
  source 1..20: mol_type = mRNA
ggcctggccccgagccccga

SEQ ID NO: 125 - RNA, 20 nt, Homo sapiens
  source 1..20: mol_type = mRNA
gcctggccccgagccccgag

SEQ ID NO: 126 - RNA, 20 nt, Homo sapiens
  source 1..20: mol_type = mRNA
cctggccccgagccccgagc

SEQ ID NO: 127 - RNA, 20 nt, Homo sapiens
  source 1..20: mol_type = mRNA
ctggccccgagccccgagcg

SEQ ID NO: 128 - RNA, 18 nt, Homo sapiens
  source 1..18: mol_type = mRNA
agctcccggagcggcctg

SEQ ID NO: 129 - RNA, 18 nt, Homo sapiens
  source 1..18: mol_type = mRNA
gctcccggagcggcctgg

SEQ ID NO: 130 - RNA, 18 nt, Homo sapiens
  source 1..18: mol_type = mRNA
ctcccggagcggcctggc

SEQ ID NO: 131 - RNA, 18 nt, Homo sapiens
  source 1..18: mol_type = mRNA
tcccggagcggcctggcc

SEQ ID NO: 132 - RNA, 18 nt, Homo sapiens
  source 1..18: mol_type = mRNA
cccggagcggcctggccc

SEQ ID NO: 133 - RNA, 18 nt, Homo sapiens
  source 1..18: mol_type = mRNA
ccggagcggcctggcccc

SEQ ID NO: 134 - RNA, 18 nt, Homo sapiens
  source 1..18: mol_type = mRNA
cggagcggcctggccccg

SEQ ID NO: 135 - RNA, 18 nt, Homo sapiens
  source 1..18: mol_type = mRNA
ggagcggcctggccccga

SEQ ID NO: 136 - RNA, 18 nt, Homo sapiens
  source 1..18: mol_type = mRNA
gagcggcctggccccgag

SEQ ID NO: 137 - RNA, 18 nt, Homo sapiens
  source 1..18: mol_type = mRNA
agcggcctggccccgagc

SEQ ID NO: 138 - RNA, 18 nt, Homo sapiens
  source 1..18: mol_type = mRNA
gcggcctggccccgagcc

SEQ ID NO: 139 - RNA, 18 nt, Homo sapiens
  source 1..18: mol_type = mRNA
cggcctggccccgagccc

SEQ ID NO: 140 - RNA, 18 nt, Homo sapiens
  source 1..18: mol_type = mRNA
ggcctggccccgagcccc

SEQ ID NO: 141 - RNA, 18 nt, Homo sapiens
  source 1..18: mol_type = mRNA
gcctggccccgagccccg

SEQ ID NO: 142 - RNA, 18 nt, Homo sapiens
  source 1..18: mol_type = mRNA
cctggccccgagccccga

SEQ ID NO: 143 - RNA, 18 nt, Homo sapiens
  source 1..18: mol_type = mRNA
ctggccccgagccccgag

SEQ ID NO: 144 - RNA, 16 nt, Homo sapiens
  source 1..16: mol_type = mRNA
ctcccggagcggcctg

SEQ ID NO: 145 - RNA, 16 nt, Homo sapiens
  source 1..16: mol_type = mRNA
tcccggagcggcctgg

SEQ ID NO: 146 - RNA, 16 nt, Homo sapiens
  source 1..16: mol_type = mRNA
cccggagcggcctggc

SEQ ID NO: 147 - RNA, 16 nt, Homo sapiens
  source 1..16: mol_type = mRNA
ccggagcggcctggcc

SEQ ID NO: 148 - RNA, 16 nt, Homo sapiens
  source 1..16: mol_type = mRNA
cggagcggcctggccc

SEQ ID NO: 149 - RNA, 16 nt, Homo sapiens
  source 1..16: mol_type = mRNA
ggagcggcctggcccc

SEQ ID NO: 150 - RNA, 16 nt, Homo sapiens
  source 1..16: mol_type = mRNA
gagcggcctggccccg

SEQ ID NO: 151 - RNA, 16 nt, Homo sapiens
  source 1..16: mol_type = mRNA
agcggcctggccccga

SEQ ID NO: 152 - RNA, 16 nt, Homo sapiens
  source 1..16: mol_type = mRNA
gcggcctggccccgag

SEQ ID NO: 153 - RNA, 16 nt, Homo sapiens
  source 1..16: mol_type = mRNA
cggcctggccccgagc

SEQ ID NO: 154 - RNA, 16 nt, Homo sapiens
  source 1..16: mol_type = mRNA
ggcctggccccgagcc

SEQ ID NO: 155 - RNA, 16 nt, Homo sapiens
  source 1..16: mol_type = mRNA
gcctggccccgagccc

SEQ ID NO: 156 - RNA, 16 nt, Homo sapiens
  source 1..16: mol_type = mRNA
cctggccccgagcccc

SEQ ID NO: 157 - RNA, 16 nt, Homo sapiens
  source 1..16: mol_type = mRNA
ctggccccgagccccg

SEQ ID NO: 158 - RNA, 16 nt, synthetic construct
  source 1..16: mol_type = other RNA
catggcgggcgcgggg

SEQ ID NO: 159 - RNA, 16 nt, synthetic construct
  source 1..16: mol_type = other RNA
catggccccgccgtcc

SEQ ID NO: 160 - RNA, 16 nt, synthetic construct
  source 1..16: mol_type = other RNA
  modified_base 1..16: mod_base = OTHER; note = 2'-O-methyl (2'-O-Me) modification to the ribose group of each nucleotide
  modified_base 1..16: mod_base = OTHER; note = Phosphorothioate (PS) linkage between adjacent nucleotides
catggtgcggcacggg

SEQ ID NO: 161 - RNA, 16 nt, synthetic construct
  source 1..16: mol_type = other RNA
  modified_base 1..16: mod_base = OTHER; note = 2'-O-methyl (2'-O-Me) modification to the ribose group in each nucleotide
  modified_base 1..16: mod_base = OTHER; note = Phosphorothioate (PS) linkages between adjacent nucleotides
cttgctgtgggaccgg

SEQ ID NO: 162 - RNA, 16 nt, synthetic construct
  source 1..16: mol_type = other RNA
  modified_base 1..16: mod_base = OTHER; note = 2'-O-methyl (2'-O-Me) modification to the ribose group in each nucleotide
  modified_base 1..16: mod_base = OTHER; note = Phosphorothioate (PS) linkage between adjacent nucleotides
catggccccggttccc

SEQ ID NO: 163 - RNA, 16 nt, synthetic construct
  source 1..16: mol_type = other RNA
  modified_base 1..16: mod_base = OTHER; note = 2'-O-methyl (2'-O-Me) modification to the ribose group in each nucleotide
  modified_base 1..16: mod_base = OTHER; note = Phosphorothioate (PS) linkage between adjacent nucleotides
cttgcccgcgcttacc

(19) United States
(12) Patent Application Publication
(10) Pub. No.: US 2025/0339561 A1
(43) Pub. Date: Nov. 6, 2025

(54) METHODS OF TREATING, AMELIORATING AND/OR PREVENTING POLYCYSTIC KIDNEY DISEASE AND POLYCYSTIC LIVER DISEASE

(71) Applicant: Yale University, New Haven, CT (US)

(72) Inventors: Whitney Besse, New Haven, CT (US); Zhigui Li, New Haven, CT (US)

(21) Appl. No.: 18/868,537

(22) PCT Filed: May 24, 2023
(86) PCT No.: PCT/US2023/023414

§ 371 Date: Nov. 22, 2024

Related U.S. Application Data
(60) Provisional application No. 63/345,634, filed on May 25, 2022.
(60) Provisional application No. 63/359,109, filed on Jul. 7, 2022.

(51) Int. Cl. A61K 48/00; A61K 31/7115; A61K 31/712; A61K 31/7125; A61P 13/12; C12N 15/113

(52) U.S. Cl. CPC A61K 48/0058; A61K 31/7115; A61K 31/712; A61K 31/7125; A61P 13/12; C12N 15/113; C12N 2310/20

(57) ABSTRACT

Described herein is a method of ameliorating and/or preventing autosomal dominant polycystic kidney disease (ADPKD) or a polycystic liver disease (PCLD) in a subject in need thereof. The method includes administering to the subject an effective amount of a compound that suppresses the translation of the first upstream open reading frame (uORF), the second uORF, the third uORF and/or the fourth uORF of the PKD1 gene.

### CROSS-REFERENCE TO RELATED APPLICATIONS

The present application claims priority under 35 U.S.C. § 119 (e) to U.S. Provisional Patent Application No. 63/345,634, filed May 25, 2022 and U.S. Provisional Patent Application No. 63/359,109, filed Jul. 7, 2022, each of which is incorporated herein by reference in its entirety.

### STATEMENT REGARDING FEDERALLY SPONSORED RESEARCH

This invention was made with government support under 1K08DK119642-01 awarded by the National Institutes of Health. The government has certain rights in the invention.

### SEQUENCE LISTING

The ASCII text file named “047162-7395WO1(02006)_Seq Listing.xml” created on May 24, 2023, comprising 173 Kbytes, is hereby incorporated by reference in its entirety.

### BACKGROUND

Autosomal dominant polycystic kidney disease (ADPKD) is a highly penetrant inherited polycystic disease which causes cysts and deformation of the kidneys, typically over the span of decades, and eventually leads to kidney failure requiring dialysis or transplantation in the majority of patients after the fifth decade of life.

Although ADPKD is sometimes considered an orphan disease, the number of ADPKD patients is in fact significant. There are estimated to be over 600,000 affected individuals with ADPKD in the US alone and over 12 million worldwide. Furthermore, since ADPKD is not subject to founder mutations but rather de novo mutations that occur all the time, the population of ADPKD patients is expected to further grow as the world population expands.

Currently, there is only one approved medication for ADPKD, tolvaptan. Unfortunately, tolvaptan has significant side effects. The drug causes polyuria (6.0±1.8 L of urine per day, Kramers et al., BMC Nephrol. 2018; 19:157), carries a black box warning (i.e., FDA's most stringent warning for drugs on the market) for hepatic injury, and is being subjected to the Risk Evaluation and Mitigation Strategy (REMS) by the FDA. Considering that many ADPKD patients require long-term treatments, the significant side effects of tolvaptan are especially undesirable. Further, even when tolerated at maximal therapeutic dose, tolvaptan therapy provides only a very modest delay in kidney failure.

Over 90% of patients with ADPKD also have multiple cysts in the liver. This is known as polycystic liver disease. These cysts occur by a similar mechanism affecting bile duct epithelium as affects kidney tubule epithelium. While liver cysts do not typically cause liver failure, a subset of patients may have debilitating symptoms from profound enlargement of the liver including pain, infection, swelling, and early satiety with inability to maintain adequate nutrition. There is no FDA-approved medical therapy for liver cysts. Instead, patients are offered repeated interventional or surgical procedures to aspirate, fenestrate, or resect cysts, or have partial hepatectomy or total hepatectomy with liver transplant. Clinically indistinguishable polycystic liver disease can occur in the absence of kidney cysts when caused by a different but related genetic mechanism. This isolated polycystic liver disease (PCLD, also referred to as “autosomal dominant polycystic liver disease” or “ADPLD”) is considered to be a rare hereditary disease, although its prevalence may approach that of ADPKD if asymptomatic cases determined at autopsy are included. About 20% of PCLD patients develop obvious clinical symptoms such as dyspnea, early satiety, abdominal distension, malnutrition, gastroesophageal reflux, and back pain, which are caused by hepatomegaly pressing surrounding organs or cyst complications. Currently, the only definitive treatment of PCLD, used in only the most severe cases, is liver transplant.

Therefore, there is a need for novel treatments of polycystic kidney disease and polycystic liver diseases. The present invention addresses this need.

### SUMMARY

In some aspects, the present invention is directed to the following:

In one aspect, the invention provides a method of treating, ameliorating and/or preventing an autosomal dominant polycystic kidney disease (ADPKD) or a polycystic liver disease (PCLD) in a subject in need thereof, comprising: administering to the subject an effective amount of a compound that suppresses the translation of a first upstream open reading frame (uORF), a second uORF, a third uORF, and/or a fourth uORF of the PKD1 gene.

In various embodiments, the method is a method of treating, ameliorating and/or preventing the ADPKD in the subject, and wherein the ADPKD is caused by or involves a mutation in the PKD1 gene in the subject.

In various embodiments, the method is a method of treating, ameliorating and/or preventing the PCLD in the subject, and wherein the PCLD is caused by or involves a germline mutation of the PKD1 gene, the PKD2 gene, the PRKCSH gene, the SEC63 gene, the GANAB gene, the ALG8 gene, the ALG9 gene, the SEC61B gene, or the DNAJB11 gene in the subject.

In various embodiments, the compound comprises:
CRISPR components that disrupt the genomic DNA sequence that encoding the first uORF, the second uORF, the third uORF, and/or the fourth uORF, or an expression vector expressing the CRISPR components; or
an antisense oligonucleotide (ASO) that blocks the translation of the first uORF, the second uORF, the third uORF, and/or the fourth uORF, or an expression vector expressing the ASO.

In various embodiments, the compound comprises the CRISPR components or the expression vector expressing the CRISPR components, and wherein the CRISPR components disrupt the initiation codon of the first uORF, the second uORF, the third uORF, and/or the fourth uORF.

In various embodiments, the compound comprises the ASO or the expression vector expressing the ASO, and wherein the portion of the PKD1 mRNA complementary to the ASO extends to 5 nucleotides away or less from the initiation codon of the first uORF, the second uORF, the third uORF, or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF, or the fourth uORF.

In various embodiments, a length of the ASO is 10 nucleotides or longer, such as 11 nucleotides or longer, 12 nucleotides or longer, 13 nucleotides or longer, 14 nucleotides or longer or 15 nucleotides or longer.

In various embodiments, a length of the ASO is 30 nucleotides or shorter, such as 29 nucleotides or shorter, 28 nucleotides or shorter, 27 nucleotides or shorter, 26 nucleotides or shorter or 25 nucleotides or shorter.

In various embodiments, at least one of the following applies:
(a) the ASO is fully complementary with one sequence set forth in SEQ ID NOs: 14-61,
(b) the ASO is fully complementary with one sequence set forth in SEQ ID NOs: 62-109,
(c) the ASO is fully complementary with one sequence set forth in SEQ ID NOs: 110-157,
(d) the ASO comprises the nucleotide sequence CAUGGGGGCGCGGGG (SEQ ID NO:158), the nucleotide sequence CAUGGCCCCGCCGUCC (SEQ ID NO: 159).

In various embodiments, the ASO comprises a modified nucleobase, a modified sugar group, or a modified linkage.

In various embodiments, at least one of the following applies:
(a) the ASO comprises the modified sugar group, and the modified sugar group comprise a 2′-O-methylation modified sugar group, such as a 2′-O-methylation modified ribose group,
(b) the ASO comprises the modified linkage, and the modified linkage comprise a phosphorothioate (PS) linkage.

In various embodiments, the subject is a mammal, such as a human.

In various embodiments, the compound comprises the ASO or the expression vector expressing the ASO, and wherein a concentration of the ASO in kidney or lung of the subject ranges from about 1 nm to about 100 nm.

In another aspect, the invention provides a method of increasing PKD1 expression in a cell, comprising:

contacting with the cell an effective amount of a compound that suppresses the translation of the first upstream open reading frame (uORF), the second uORF, the third uORF, and/or the fourth uORF of the PKD1 gene.

In various embodiments, the cell has a mutation in the PKD1 gene, the PKD2 gene, the PRKCSH gene, the SEC63 gene, the GANAB gene, the ALG8 gene, the ALG9 gene, the SEC61B gene, or the DNAJB11 gene.

In various embodiments, the cell is in a tissue or a subject.

In various embodiments, the cell is a kidney cell in a subject diagnosed with autosomal dominant polycystic kidney disease (ADPKD) or a liver cell in a subject diagnosed with polycystic liver disease (PCLD).

In various embodiments, the compound comprises:
CRISPR components that disrupt the genomic DNA sequence that encoding the first uORF, the second uORF, the third uORF and/or the fourth uORF, or an expression vector expressing the CRISPR components; or
an antisense oligonucleotide (ASO) that blocks the first uORF, the second uORF, the third uORF and/or the fourth uORF, or an expression vector expressing the ASO.

In various embodiments, the compound comprises the CRISPR components or the expression vector expressing the CRISPR components, and wherein the CRISPR components disrupt the initiation codon of the first uORF, the second uORF, the third uORF and/or the fourth uORF.

In various embodiments, the compound comprises the ASO or the expression vector expressing the ASO, and wherein the portion of the PKD1 mRNA complementary to the ASO extends to 5 nucleotides away or less from the initiation codon of the first uORF, the second uORF, the third uORF or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF or the fourth uORF.

In various embodiments, a length of the ASO is 10 nucleotides or longer, such as 11 nucleotides or longer, 12 nucleotides or longer, 13 nucleotides or longer, 14 nucleotides or longer or 15 nucleotides or longer.

In various embodiments, a length of the ASO is 30 nucleotides or shorter, such as 29 nucleotides or shorter, 28 nucleotides or shorter, 27 nucleotides or shorter, 26 nucleotides or shorter or 25 nucleotides or shorter.

In various embodiments, at least one of the following applies:
(a) the ASO is fully complementary with one sequence set forth in SEQ ID NOs: 14-61,
(b) the ASO is fully complementary with one sequence set forth in SEQ ID NOs: 62-109,
(c) the ASO is fully complementary with one sequence set forth in SEQ ID NOs: 110-157,
(d) the ASO comprises the nucleotide sequence CAUGGCGGGCGCGGGG (SEQ ID NO:158), the nucleotide sequence CAUGGCCCCGCCGUCC (SEQ ID NO: 159).

In various embodiments, the ASO comprises a modified nucleobase, a modified sugar group or a modified linkage.

In various embodiments, at least one of the following applies:
(a) the ASO comprises the modified sugar, and the modified sugar group comprise a 2′-O-methylation modified sugar group, such as a 2′-O-methylation modified ribose group,
(b) the ASO comprises the modified linkage, and the modified linkage comprise a phosphorothioate (PS) linkage.

In various embodiments, the compound comprises the ASO or the expression vector expressing the ASO, and wherein a concentration of the ASO contacted with the cell ranges from about 1 nm to about 100 nm.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1C demonstrate that decreased functional dosage of PKD1/Polycystin-1 (PC1), either from loss-of-function mutations of the PKD1 gene per se or mutations of endoplasmic reticulum (ER) genes responsible for the maturation of PC1, result in renal cyst, which can be rescued by increasing Pkd1 copy numbers, in accordance with some embodiments. FIGS. 1A-1B: MRIs of severe polycystic disease in patients with PKD1 non-truncating mutation (kidney and liver) (FIG. 1A) or SEC63 mutation (liver only) (FIG. 1B). FIG. 1C: PC1 dosage-dependent mouse model at age 3 weeks. PC1 matures inefficiently in the ER in the absence of Sec63, but in this setting the resultant cyst formation can be prevented by producing more PC1 from 3 extra genomic copies of Pkd1 (Pkd1^{F/H}-BAC). Ksp-Cre is active in distal nephron from mid-embryogenesis.

FIGS. 2A-2B illustrate certain aspects of the 5′UTR and uORFs of human PKD1, in accordance with some embodiments. FIG. 2A: Linear sequence of the start of PKD1 mRNA sequence, showing the uORF locations in the 5′UTR. FIG. 2B: Predicted secondary structure of PKD1 5′UTR. FIG. 2A shows nucleotides 1-300 of SEQ ID NO: 1, which includes the coding sequence for the first 30 amino acid residues of human PC-1 protein (MPPAAPARLALALGLGLWLGALAGGPGRGC, SEQ ID NO:11). FIG. 2B shows nucleotides 1-209 of SEQ ID NO: 1.

FIGS. 3A-3B illustrate certain aspects of how uORFs function, as well how antisense oligonucleotides (ASOs) inhibit uORFs, in accordance with some embodiments. FIG. 3A: Illustration of deleterious effect of uORFs on translation of protein. FIG. 3B: ASOs preventing translation of uORFs to allow increased protein translation.

FIGS. 4A-4D demonstrate that the uORFs in the 5′UTR of human PKD1 downregulates translation and that the disruption of the uORFs abolishes this downregulation, in accordance with some embodiments. FIG. 4A: A dual luciferase reporter construct in which the 5′UTR of Renilla luciferase was replaced with either wild-type 5′UTR or variations of the human PKD1 5′UTR with mutant uORF. FIG. 4B: Sequences of the wildtype uORF (“wt”) and mutant uORFs which containing either one (“ΔuORF1” and “ΔuORF2”) or two (“ΔuORF1&2”) single base edits to abolish either one or both of the uORFs). In FIG. 4B, ΔuORF1 means an A to T mutation in nucleotide residue 123 of SEQ ID NO:2, ΔuORF2 means an A to T mutation in nucleotide residue 190 of SEQ ID NO:2, and ΔuORF1&2 means both of the A to T mutations are included. FIG. 4C: The targeted edits of PKD1 5′UTR resulted in significant increased expression of the luciferase protein. Notably, abolishing both the uORFs increased the protein expression by nearly 4-fold (“1&2” vs “wt”). FIG. 4D: the mutations in the uORFs did not alter the mRNA expression.

FIG. 5 demonstrates that antisense oligonucleotides (ASOs) inhibiting the uORFs in the 5′UTR of PKD1 increases PC1 protein expression, in accordance with some embodiments. After transfection of cells expressing the wt-uORF luciferase reporter (FIGS. 4A-4D), nucleotide ASOs complementary to the mRNA sequence upstream and overlapping with the ATG of uORF initiation codon for uORF1, uORF2, or both, or mismatched controls (MM1, MM2) ASOs were incubated with the transfected cells. Lysate collected after 24 hours of treatment showed a significant increase in luciferase expression (approximately 3-fold increase) when both uORF1&2 were targeted.

FIGS. 6A-6G demonstrate that ASO treatment in culture media of human epithelial cells significantly increased the protein expression of PC1, in accordance with some embodiments. FIG. 6A: ASO1, ASO2 and combination thereof increase PC1 steady state expression without effecting mRNA expression. Protein expression data in this figure is from 48 hour timepoint which is also included in FIG. 6D. FIGS. 6B-6D: the expression increase of PC1 protein that is achieved by treatment with ASO1, ASO2, or their combination is most significant when the treatment is maintained for longer (expression level normalized to loading control Vinculin on western blot shows 48 and 96 hour timepoints have strongest effect). Of note, fresh media containing the prescribed concentration of ASO was applied once daily. FIG. 6E-6G various dosages of ASO1 and ASO2 produced the expression increase effect in the cultured epithelial cells.

FIG. 7 demonstrates that mouse polycystic kidney disease caused by epithelial cell specific loss of the human polycystic kidney disease gene DNAJB11 is sensitive to 50% reduction in PC1 dosage, in accordance with some embodiments. This genetic interaction supports the hypothesis that a 2-fold increase in PC1 expression, as achieved in in vitro preliminary data could be sufficient to not only slow but prevent cyst formation entirely.

FIG. 8 shows uORF1 and uORF2 in the mouse Pkd1 mRNA 5′UTR, in accordance with some embodiments. Mouse Pkd1 5′UTR contains two ATG-initiated potential uORFs corresponding to those found in the human sequence. These two uORFs were termed mouse uORF1 and mouse uORF2, in consistence with the human uORFs described above. FIG. 8 shows residues 1-360 of SEQ ID NO:7, which includes the coding sequence for the first 16 amino acid residues of the mouse PC1 protein (MPLGAPALLALALGLG, SEQ ID NO: 12).

FIG. 9 shows the design of ASOs for sterically blocking mouse Pkd1 uORF translation, in accordance with some embodiments. FIG. 9 shows nucleotide residues 1-316 of SEQ ID NO:7.

FIGS. 10A-10B: PC1 protein expression upon treatment with ASOs targeting mouse Pkd1 uORFs in a mouse cell line that contains an HA epitope tag on the C-terminus of Pkd1 to allow for assessment of the C-terminal fragment, in accordance with some embodiments. A concentration of 20 nM were used for all the ASOs, assessment after (FIG. 10A) 24 h or (FIG. 10B) 96 hours post-administration.

FIG. 11: PC1 protein expression upon treatment with ASOs targeting mouse Pkd1 uORFs in a mouse cell line that contains a missense mutation p.R2216W (orthologue of human variant p.R2220W) and a V5 epitope tag on the C-terminus of Pkd1 to allow for assessment of the C-terminal fragment, in accordance with some embodiments. A concentration of 20 nM were used for all the ASOs; assessment after 96 hours post-administration. The data demonstrate that that even in the setting of a pathogenic missense variant, inhibition of Pkd1 uORFs significantly increased the production of the mature protein (PC1 C-terminal fragment).

FIG. 12 shows the location of 3 sites of translation initiation seen in publicly available ribosome profiling experiments, as visualized by the publicly available Ribo-uORF resource. The uORF with the evidence of translation in the most experiments was our uORF1. Two additional uORFs (uORF3 and uORF4) with CTG initiation codons in the Human PKD1 mRNA 5′-UTR were also identified with evidence from at least one ribosome profiling dataset. These could be targeted by ASO to increase the PC1 protein expression, in accordance with some embodiments. The sequence shown in FIG. 12 is the nucleotide residues 1-306 of SEQ ID NO: 1 (with the DNA nucleotide T in place of the RNA nucleotide U). The uORF numbering has been edited from the website's default to be consistent with our numbering (Ref: Liu et al. Nucleic Acids Research 2023).

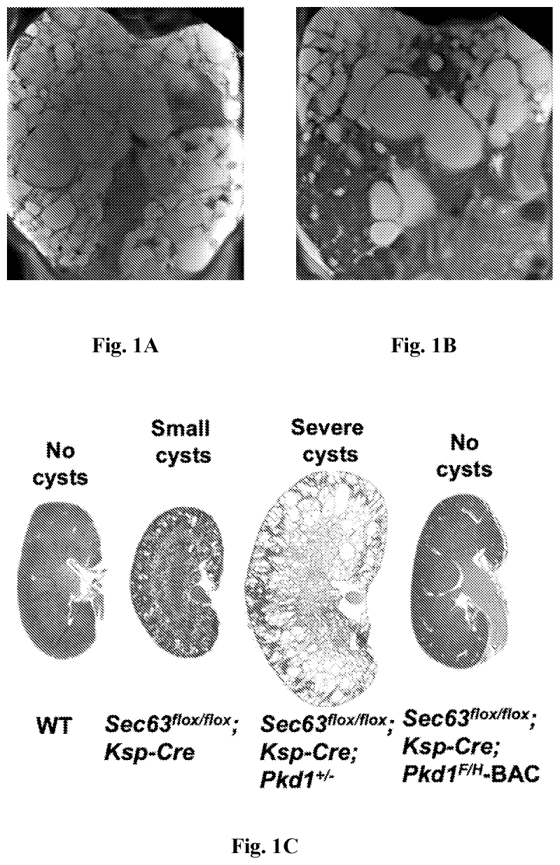

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

PCLD, as well as a significant subset of ADPKD are caused by insufficient PC1 functional dosage, which are caused by either loss-of-function mutations of the PKD1 gene per se, or mutations of endoplasmic reticulum (ER) genes responsible for sorting PKD1 to the cell surface of the primary cilium.

It was hypothesized that both PCLD and ADPKD caused by insufficient PC1 functional dosage can be treated, ameliorated and/or prevented by increase the functional dosage of PC1 in the patient. The only known method to increase PC1 expression is the inhibition of microRNA 17 (Lakhia et al., “PKD1 and PKD2 mRNA cis-inhibition drives polycystic kidney disease progression”). The basis of that proposed therapy—whose investigation is also at the level of mouse model pre-clinical investigations—is to block what the authors propose is steady-state inhibition of PKD1 mRNA by micro RNA 17 binding to the 3′-UTR of PKD1 transcripts. The in vivo studies show promise for the approach of therapeutically increasing PC1 dosage. Since the complementary sequence to microRNA 17 sequence is found in a large number of genes, treating PCLD and ADPKD with this molecule is expected to have significant and unpredictable burden of off-target effects, i.e. lack of specificity.

The present study discovered four upstream open reading frames (uORFs) in the 5′-untranslated region (5′-UTR) of the PKD1 messenger RNA (mRNA). The present study further discovered that translation of uORF1 and/or uORF2 results in reduced translation of PKD1 mRNA into PC1 protein, and that either abolishing these two uORFs through mutations or blocking translation of the uORFs using antisense oligonucleotides (ASOs) significantly enhanced the translation of the PKD1 mRNA and thereby increased the protein level of PC1. It is expected that the third and the fourth uORFs may function similarly or complementary to the first and the second uORF, and that the abolishment and/or suppression of the third and the fourth uORFs may achieve similar or enhanced results. Since the uORFs in the 5′-UTR of PKD1 have unique sequences, and methods targeting the uORFs sequences, such as CRISPR or ASO, can be designed with high specificity, treatments of ADPKD or PCLD through the uORFs are expected to be highly specific.

Accordingly, in some aspects, the present invention is directed to a method of treating, ameliorating and/or preventing a polycystic kidney disease or a polycystic liver disease.

In some aspects, the present invention is directed to a method to increase the functional dosage of PC1 in a cell, a tissue or a subject.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

As used herein “PKD1” refers to the human gene which is transcribed to produce the mRNA product represented by SEQ ID NO: 1, all the human genes at the same allele as the human gene that produces mRNA of SEQ ID NO: 1; all the ortholog genes in non-human species, as well as all the mRNA products and protein products (which are sometimes referred to as “polycystin-1”, “polycystin 1”, “PC-1,” or “PC1” protein both in the art and herein) of the human and non-human genes.

Homo sapiens polycystin 1, transient receptor potential channel
interacting (PKD1), mRNA (SEQ ID NO: 1)
GCACUGCAGCGCCAGCGUCCGAGCGGGCGGCCGAGCUCCCGGAGCGGCCUG
GCCCCGAGCCCCGAGCGGGCGUCGCUCAGCAGCAGGUCGCGGCCGCAGCCC
CAUCCAGCCCCGCGCCCGCCAUGCCGUCCGCGGGCCCCGCCUGAGCUGCGG
CCUCCGCGCGCGGGCGGGCCUGGGGACGGCGGGGCCAUGCGCGCGCUGCCC
UAACGAUGCCGCCCGCCGCGCCCGCCCGCCUGGCGCUGGCCCUGGGCCUGG
GCCUGUGGCUCGGGGCGCUGGCGGGGGGCCCCGGGCGCGGCUGCGGGCCCU
GCGAGCCCCCCUGCCUCUGCGGCCCAGCGCCCGGCGCCGCCUGCCGCGUCA
ACUGCUCGGGCCGCGGGCUGCGGACGCUCGGUCCCGCGCUGCGCAUCCCCG
CGGACGCCACAGCGCUAGACGUCUCCCACAACCUGCUCCGGGCGCUGGACG
UUGGGCUCCUGGCGAACCUCUCGGCGCUGGCAGAGCUGGAUAUAAGCAAC
AACAAGAUUUCUACGUUAGAAGAAGGAAUAUUUGCUAAUUUAUUUAAUUU
AAGUGAAAUAAACCUGAGUGGGAACCCGUUUGAGUGUGACUGUGGCCUGG
CGUGGCUGCCGCGAUGGGCGGAGGAGCAGCAGGUGCGGGUGGUGCAGCCC
GAGGCAGCCACGUGUGCUGGGCCUGGCUCCCUGGCUGGCCAGCCUCUGCUU
GGCAUCCCCUUGCUGGACAGUGGCUGUGGUGAGGAGUAUGUCGCCUGCCU
CCCUGACAACAGCUCAGGCACCGUGGCAGCAGUGUCCUUUUCAGCUGCCCA
CGAAGGCCUGCUUCAGCCAGAGGCCUGCAGCGCCUUCUGCUUCUCCACCGG
CCAGGGCCUCGCAGCCCUCUCGGAGCAGGGCUGGUGCCUGUGUGGGGCGGC
CCAGCCCUCCAGUGCCUCCUUUGCCUGCCUGUCCCUCUGCUCCGGCCCCCC
GCCACCUCCUGCCCCCACCUGUAGGGGCCCCACCCUCCUCCAGCACGUCUU
CCCUGCCUCCCCAGGGGCCACCCUGGUGGGGCCCCACGGACCUCUGGCCUC
UGGCCAGCUAGCAGCCUUCCACAUCGCUGCCCCGCUCCCUGUCACUGCCAC
ACGCUGGGACUUCGGAGACGGCUCCGCCGAGGUGGAUGCCGCUGGGCCGG
CUGCCUCGCAUCGCUAUGUGCUGCCUGGGCGCUAUCACGUGACGGCCGUGC
UGGCCCUGGGGGCCGGCUCAGCCCUGCUGGGGACAGACGUGCAGGUGGAA
GCGGCACCUGCCGCCCUGGAGCUCGUGUGCCCGUCCUCGGUGCAGAGUGAC
GAGAGCCUCGACCUCAGCAUCCAGAACCGCGGUGGUUCAGGCCUGGAGGCC
GCCUACAGCAUCGUGGCCCUGGGCGAGGAGCCGGCCCGAGCGGUGCACCCG
CUCUGCCCCUCGGACACGGAGAUCUUCCCUGGCAACGGGCACUGCUACCGC
CUGGUGGUGGAGAAGGCGGCCUGGCUGCAGGCGCAGGAGCAGUGUCAGGC
CUGGGCCGGGGCCGCCCUGGCAAUGGUGGACAGUCCCGCCGUGCAGCGCUU
CCUGGUCUCCCGGGUCACCAGGAGCCUAGACGUGUGGAUCGGCUUCUCGAC
UGUGCAGGGGGUGGAGGUGGGCCCAGCGCCGCAGGGCGAGGCCUUCAGCC
UGGAGAGCUGCCAGAACUGGCUGCCCGGGGAGCCACACCCAGCCACAGCCG
AGCACUGCGUCCGGCUCGGGCCCACCGGGUGGUGUAACACCGACCUGUGCU
CAGCGCCGCACAGCUACGUCUGCGAGCUGCAGCCCGGAGGCCCAGUGCAGG
AUGCCGAGAACCUCCUCGUGGGAGCGCCCAGUGGGGACCUGCAGGGACCCC
UGACGCCUCUGGCACAGCAGGACGGCCUCUCAGCCCCGCACGAGCCCGUGG
AGGUCAUGGUAUUCCCGGGCCUGCGUCUGAGCCGUGAAGCCUUCCUCACCA
CGGCCGAAUUUGGGACCCAGGAGCUCCGGCGGCCCGCCCAGCUGCGGCUGC
AGGUGUACCGGCUCCUCAGCACAGCAGGGACCCCGGAGAACGGCAGCGAGC
CUGAGAGCAGGUCCCCGGACAACAGGACCCAGCUGGCCCCCGCGUGCAUGC
CAGGGGGACGCUGGUGCCCUGGAGCCAACAUCUGCUUGCCGCUGGACGCCU
CCUGCCACCCCCAGGCCUGCGCCAAUGGCUGCACGUCAGGGCCAGGGCUAC
CCGGGGCCCCCUAUGCGCUAUGGAGAGAGUUCCUCUUCUCCGUUCCCGCGG
GGCCCCCCGCGCAGUACUCGGUCACCCUCCACGGCCAGGAUGUCCUCAUGC
UCCCUGGUGACCUCGUUGGCUUGCAGCACGACGCUGGCCCUGGCGCCCUCC
UGCACUGCUCGCCGGCUCCCGGCCACCCUGGUCCCCGGGCCCCGUACCUCU
CCGCCAACGCCUCGUCAUGGCUGCCCCACUUGCCAGCCCAGCUGGAGGGCA
CUUGGGCCUGCCCUGCCUGUGCCCUGCGGCUGCUUGCAGCCACGGAACAGC
UCACCGUGCUGCUGGGCUUGAGGCCCAACCCUGGACUGCGGCUGCCUGGGC
GCUAUGAGGUCCGGGCAGAGGUGGGCAAUGGCGUGUCCAGGCACAACCUC
UCCUGCAGCUUUGACGUGGUCUCCCCAGUGGCUGGGCUGCGGGUCAUCUA
CCCUGCCCCCCGCGACGGCCGCCUCUACGUGCCCACCAACGGCUCAGCCUU
GGUGCUCCAGGUGGACUCUGGUGCCAACGCCACGGCCACGGCUCGCUGGCC
UGGGGGCAGUGUCAGCGCCCGCUUUGAGAAUGUCUGCCCUGCCCUGGUGG
CCACCUUCGUGCCCGGCUGCCCCUGGGAGACCAACGAUACCCUGUUCUCAG
UGGUAGCACUGCCGUGGCUCAGUGAGGGGGAGCACGUGGUGGACGUGGUG
GUGGAAAACAGCGCCAGCCGGGCCAACCUCAGCCUGCGGGUGACGGCGGA
GGAGCCCAUCUGUGGCCUCCGCGCCACGCCCAGCCCCGAGGCCCGUGUACU
GCAGGGAGUCCUAGUGAGGUACAGCCCCGUGGUGGAGGCCGGCUCGGACA
UGGUCUUCCGGUGGACCAUCAACGACAAGCAGUCCCUGACCUUCCAGAACG
UGGUCUUCAAUGUCAUUUAUCAGAGCGCGGCGGUCUUCAAGCUCUCACUG
ACGGCCUCCAACCACGUGAGCAACGUCACCGUGAACUACAACGUAACCGUG
GAGCGGAUGAACAGGAUGCAGGGUCUGCAGGUCUCCACAGUGCCGGCCGU
GCUGUCCCCCAAUGCCACGCUAGCACUGACGGCGGGCGUGCUGGUGGACUC
GGCCGUGGAGGUGGCCUUCCUGUGGACCUUUGGGGAUGGGGAGCAGGCCC
UCCACCAGUUCCAGCCUCCGUACAACGAGUCCUUCCCGGUUCCAGACCCCU
CGGUGGCCCAGGUGCUGGUGGAGCACAAUGUCAUGCACACCUACGCUGCCC
CAGGUGAGUACCUCCUGACCGUGCUGGCAUCUAAUGCCUUCGAGAACCUG
ACGCAGCAGGUGCCUGUGAGCGUGCGCGCCUCCCUGCCCUCCGUGGCUGUG
GGUGUGAGUGACGGCGUCCUGGUGGCCGGCCGGCCCGUCACCUUCUACCCG
CACCCGCUGCCCUCGCCUGGGGGUGUUCUUUACACGUGGGACUUCGGGGAC
GGCUCCCCUGUCCUGACCCAGAGCCAGCCGGCUGCCAACCACACCUAUGCC
UCGAGGGGCACCUACCACGUGCGCCUGGAGGUCAACAACACGGUGAGCGG
UGCGGCGGCCCAGGCGGAUGUGCGCGUCUUUGAGGAGCUCCGCGGACUCA
GCGUGGACAUGAGCCUGGCCGUGGAGCAGGGCGCCCCCGUGGUGGUCAGC
GCCGCGGUGCAGACGGGCGACAACAUCACGUGGACCUUCGACAUGGGGGA
CGGCACCGUGCUGUCGGGCCCGGAGGCAACAGUGGAGCAUGUGUACCUGC
GGGCACAGAACUGCACAGUGACCGUGGGUGCGGCCAGCCCCGCCGGCCACC
UGGCCCGGAGCCUGCACGUGCUGGUCUUCGUCCUGGAGGUGCUGCGCGUU
GAACCCGCCGCCUGCAUCCCCACGCAGCCUGACGCGCGGCUCACGGCCUAC
GUCACCGGGAACCCGGCCCACUACCUCUUCGACUGGACCUUCGGGGAUGGC
UCCUCCAACACGACCGUGCGGGGGUGCCCGACGGUGACACACAACUUCACG
CGGAGCGGCACGUUCCCCCUGGCGCUGGUGCUGUCCAGCCGCGUGAACAGG
GCGCAUUACUUCACCAGCAUCUGCGUGGAGCCAGAGGUGGGCAACGUCAC
CCUGCAGCCAGAGAGGCAGUUUGUGCAGCUCGGGGACGAGGCCUGGCUGG
UGGCAUGUGCCUGGCCCCCGUUCCCCUACCGCUACACCUGGGACUUUGGCA
CCGAGGAAGCCGCCCCCACCCGUGCCAGGGGCCCUGAGGUGACGUUCAUCU
ACCGAGACCCAGGCUCCUAUCUUGUGACAGUCACCGCGUCCAACAACAUCU
CUGCUGCCAAUGACUCAGCCCUGGUGGAGGUGCAGGAGCCCGUGCUGGUC
ACCAGCAUCAAGGUCAAUGGCUCCCUUGGGCUGGAGCUGCAGCAGCCGUA
CCUGUUCUCUGCUGUGGGCCGUGGGCGCCCCGCCAGCUACCUGUGGGAUCU
GGGGGACGGUGGGUGGCUCGAGGGUCCGGAGGUCACCCACGCUUACAACA
GCACAGGUGACUUCACCGUUAGGGUGGCCGGCUGGAAUGAGGUGAGCCGC
AGCGAGGCCUGGCUCAAUGUGACGGUGAAGCGGCGCGUGCGGGGGCUCGU
CGUCAAUGCAAGCCGCACGGUGGUGCCCCUGAAUGGGAGCGUGAGCUUCA
GCACGUCGCUGGAGGCCGGCAGUGAUGUGCGCUAUUCCUGGGUGCUCUGU
GACCGCUGCACGCCCAUCCCUGGGGGUCCUACCAUCUCUUACACCUUCCGC
UCCGUGGGCACCUUCAAUAUCAUCGUCACGGCUGAGAACGAGGUGGGCUC
CGCCCAGGACAGCAUCUUCGUCUAUGUCCUGCAGCUCAUAGAGGGGCUGC
AGGUGGUGGGCGGUGGCCGCUACUUCCCCACCAACCACACGGUACAGCUGC
AGGCCGUGGUUAGGGAUGGCACCAACGUCUCCUACAGCUGGACUGCCUGG
AGGGACAGGGGCCCGGCCCUGGCCGGCAGCGGCAAAGGCUUCUCGCUCACC
GUGCUCGAGGCCGGCACCUACCAUGUGCAGCUGCGGGCCACCAACAUGCUG
GGCAGCGCCUGGGCCGACUGCACCAUGGACUUCGUGGAGCCUGUGGGGUG
GCUGAUGGUGGCCGCCUCCCCGAACCCAGCUGCCGUCAACACAAGCGUCAC
CCUCAGUGCCGAGCUGGCUGGUGGCAGUGGUGUCGUAUACACUUGGUCCU
UGGAGGAGGGGCUGAGCUGGGAGACCUCCGAGCCAUUUACCACCCAUAGC
UUCCCCACACCCGGCCUGCACUUGGUCACCAUGACGGCAGGGAACCCGCUG
GGCUCAGCCAACGCCACCGUGGAAGUGGAUGUGCAGGUGCCUGUGAGUGG
CCUCAGCAUCAGGGCCAGCGAGCCCGGAGGCAGCUUCGUGGCGGCCGGGUC
CUCUGUGCCCUUUUGGGGGCAGCUGGCCACGGGCACCAAUGUGAGCUGGU
GCUGGGCUGUGCCCGGCGGCAGCAGCAAGCGUGGCCCUCAUGUCACCAUGG
UCUUCCCGGAUGCUGGCACCUUCUCCAUCCGGCUCAAUGCCUCCAACGCAG
UCAGCUGGGUCUCAGCCACGUACAACCUCACGGCGGAGGAGCCCAUCGUGG
GCCUGGUGCUGUGGGCCAGCAGCAAGGUGGUGGCGCCCGGGCAGCUGGUC
CAUUUUCAGAUCCUGCUGGCUGCCGGCUCAGCUGUCACCUUCCGCCUGCAG
GUCGGCGGGGCCAACCCCGAGGUGCUCCCCGGGCCCCGUUUCUCCCACAGC
UUCCCCCGCGUCGGAGACCACGUGGUGAGCGUGCGGGGCAAAAACCACGU
GAGCUGGGCCCAGGCGCAGGUGCGCAUCGUGGUGCUGGAGGCCGUGAGUG
GGCUGCAGGUGCCCAACUGCUGCGAGCCUGGCAUCGCCACGGGCACUGAGA
GGAACUUCACAGCCCGCGUGCAGCGCGGCUCUCGGGUCGCCUACGCCUGGU
ACUUCUCGCUGCAGAAGGUCCAGGGCGACUCGCUGGUCAUCCUGUCGGGCC
GCGACGUCACCUACACGCCCGUGGCCGCGGGGCUGUUGGAGAUCCAGGUGC
GCGCCUUCAACGCCCUGGGCAGUGAGAACCGCACGCUGGUGCUGGAGGUU
CAGGACGCCGUCCAGUAUGUGGCCCUGCAGAGCGGCCCCUGCUUCACCAAC
CGCUCGGCGCAGUUUGAGGCCGCCACCAGCCCCAGCCCCCGGCGUGUGGCC
UACCACUGGGACUUUGGGGAUGGGUCGCCAGGGCAGGACACAGAUGAGCC
CAGGGCCGAGCACUCCUACCUGAGGCCUGGGGACUACCGCGUGCAGGUGA
ACGCCUCCAACCUGGUGAGCUUCUUCGUGGCGCAGGCCACGGUGACCGUCC
AGGUGCUGGCCUGCCGGGAGCCGGAGGUGGACGUGGUCCUGCCCCUGCAG
GUGCUGAUGCGGCGAUCACAGCGCAACUACUUGGAGGCCCACGUUGACCU
GCGCGACUGCGUCACCUACCAGACUGAGUACCGCUGGGAGGUGUAUCGCA
CCGCCAGCUGCCAGCGGCCGGGGCGCCCAGCGCGUGUGGCCCUGCCCGGCG
UGGACGUGAGCCGGCCUCGGCUGGUGCUGCCGCGGCUGGCGCUGCCUGUG
GGGCACUACUGCUUUGUGUUUGUCGUGUCAUUUGGGGACACGCCACUGAC
ACAGAGCAUCCAGGCCAAUGUGACGGUGGCCCCCGAGCGCCUGGUGCCCAU
CAUUGAGGGUGGCUCAUACCGCGUGUGGUCAGACACACGGGACCUGGUGC
UGGAUGGGAGCGAGUCCUACGACCCCAACCUGGAGGACGGCGACCAGACG
CCGCUCAGUUUCCACUGGGCCUGUGUGGCUUCGACACAGAGGGAGGCUGG
CGGGUGUGCGCUGAACUUUGGGCCCCGCGGGAGCAGCACGGUCACCAUUCC
ACGGGAGCGGCUGGCGGCUGGCGUGGAGUACACCUUCAGCCUGACCGUGU
GGAAGGCCGGCCGCAAGGAGGAGGCCACCAACCAGACGGUGCUGAUCCGG
AGUGGCCGGGUGCCCAUUGUGUCCUUGGAGUGUGUGUCCUGCAAGGCACA
GGCCGUGUACGAAGUGAGCCGCAGCUCCUACGUGUACUUGGAGGGCCGCU
GCCUCAAUUGCAGCAGCGGCUCCAAGCGAGGGCGGUGGGCUGCACGUACG
UUCAGCAACAAGACGCUGGUGCUGGAUGAGACCACCACAUCCACGGGCAG
UGCAGGCAUGCGACUGGUGCUGCGGCGGGGCGUGCUGCGGGACGGCGAGG
GAUACACCUUCACGCUCACGGUGCUGGGCCGCUCUGGCGAGGAGGAGGGC
UGCGCCUCCAUCCGCCUGUCCCCCAACCGCCCGCCGCUGGGGGGCUCUUGC
CGCCUCUUCCCACUGGGCGCUGUGCACGCCCUCACCACCAAGGUGCACUUC
GAAUGCACGGGCUGGCAUGACGCGGAGGAUGCUGGCGCCCCGCUGGUGUA
CGCCCUGCUGCUGCGGCGCUGUCGCCAGGGCCACUGCGAGGAGUUCUGUGU
CUACAAGGGCAGCCUCUCCAGCUACGGAGCCGUGCUGCCCCCGGGUUUCAG
GCCACACUUCGAGGUGGGCCUGGCCGUGGUGGUGCAGGACCAGCUGGGAG
CCGCUGUGGUCGCCCUCAACAGGUCUUUGGCCAUCACCCUCCCAGAGCCCA
ACGGCAGCGCAACGGGGCUCACAGUCUGGCUGCACGGGCUCACCGCUAGUG
UGCUCCCAGGGCUGCUGCGGCAGGCCGAUCCCCAGCACGUCAUCGAGUACU
CGUUGGCCCUGGUCACCGUGCUGAACGAGUACGAGCGGGCCCUGGACGUG
GCGGCAGAGCCCAAGCACGAGCGGCAGCACCGAGCCCAGAUACGCAAGAAC
AUCACGGAGACUCUGGUGUCCCUGAGGGUCCACACUGUGGAUGACAUCCA
GCAGAUCGCUGCUGCGCUGGCCCAGUGCAUGGGGCCCAGCAGGGAGCUCG
UAUGCCGCUCGUGCCUGAAGCAGACGCUGCACAAGCUGGAGGCCAUGAUG
CUCAUCCUGCAGGCAGAGACCACCGCGGGCACCGUGACGCCCACCGCCAUC
GGAGACAGCAUCCUCAACAUCACAGGAGACCUCAUCCACCUGGCCAGCUCG
GACGUGCGGGCACCACAGCCCUCAGAGCUGGGAGCCGAGUCACCAUCUCGG
AUGGUGGCGUCCCAGGCCUACAACCUGACCUCUGCCCUCAUGCGCAUCCUC
AUGCGCUCCCGCGUGCUCAACGAGGAGCCCCUGACGCUGGCGGGCGAGGAG
AUCGUGGCCCAGGGCAAGCGCUCGGACCCGCGGAGCCUGCUGUGCUAUGGC
GGCGCCCCAGGGCCUGGCUGCCACUUCUCCAUCCCCGAGGCUUUCAGCGGG
GCCCUGGCCAACCUCAGUGACGUGGUGCAGCUCAUCUUUCUGGUGGACUCC
AAUCCCUUUCCCUUUGGCUAUAUCAGCAACUACACCGUCUCCACCAAGGUG
GCCUCGAUGGCAUUCCAGACACAGGCCGGCGCCCAGAUCCCCAUCGAGCGG
CUGGCCUCAGAGCGCGCCAUCACCGUGAAGGUGCCCAACAACUCGGACUGG
GCUGCCCGGGGCCACCGCAGCUCCGCCAACUCCGCCAACUCCGUUGUGGUC
CAGCCCCAGGCCUCCGUCGGUGCUGUGGUCACCCUGGACAGCAGCAACCCU
GCGGCCGGGCUGCAUCUGCAGCUCAACUAUACGCUGCUGGACGGCCACUAC
CUGUCUGAGGAACCUGAGCCCUACCUGGCAGUCUACCUACACUCGGAGCCC
CGGCCCAAUGAGCACAACUGCUCGGCUAGCAGGAGGAUCCGCCCAGAGUCA
CUCCAGGGUGCUGACCACCGGCCCUACACCUUCUUCAUUUCCCCGGGGAGC
AGAGACCCAGCGGGGAGUUACCAUCUGAACCUCUCCAGCCACUUCCGCUGG
UCGGCGCUGCAGGUGUCCGUGGGCCUGUACACGUCCCUGUGCCAGUACUUC
AGCGAGGAGGACAUGGUGUGGCGGACAGAGGGGCUGCUGCCCCUGGAGGA
GACCUCGCCCCGCCAGGCCGUCUGCCUCACCCGCCACCUCACCGCCUUCGG
CGCCAGCCUCUUCGUGCCCCCAAGCCAUGUCCGCUUUGUGUUUCCUGAGCC
GACAGCGGAUGUAAACUACAUCGUCAUGCUGACAUGUGCUGUGUGCCUGG
UGACCUACAUGGUCAUGGCCGCCAUCCUGCACAAGCUGGACCAGUUGGAU
GCCAGCCGGGGCCGCGCCAUCCCUUUCUGUGGGCAGCGGGGCCGCUUCAAG
UACGAGAUCCUCGUCAAGACAGGCUGGGGCCGGGGCUCAGGUACCACGGC
CCACGUGGGCAUCAUGCUGUAUGGGGUGGACAGCCGGAGCGGCCACCGGC
ACCUGGACGGCGACAGAGCCUUCCACCGCAACAGCCUGGACAUCUUCCGGA
UCGCCACCCCGCACAGCCUGGGUAGCGUGUGGAAGAUCCGAGUGUGGCAC
GACAACAAAGGGCUCAGCCCUGCCUGGUUCCUGCAGCACGUCAUCGUCAGG
GACCUGCAGACGGCACGCAGCGCCUUCUUCCUGGUCAAUGACUGGCUUUCG
GUGGAGACGGAGGCCAACGGGGGCCUGGUGGAGAAGGAGGUGCUGGCCGC
GAGCGACGCAGCCCUUUUGCGCUUCCGGCGCCUGCUGGUGGCUGAGCUGCA
GCGUGGCUUCUUUGACAAGCACAUCUGGCUCUCCAUAUGGGACCGGCCGCC
UCGUAGCCGUUUCACUCGCAUCCAGAGGGCCACCUGCUGCGUUCUCCUCAU
CUGCCUCUUCCUGGGCGCCAACGCCGUGUGGUACGGGGCUGUUGGCGACUC
UGCCUACAGCACGGGGCAUGUGUCCAGGCUGAGCCCGCUGAGCGUCGACAC
AGUCGCUGUUGGCCUGGUGUCCAGCGUGGUUGUCUAUCCCGUCUACCUGG
CCAUCCUUUUUCUCUUCCGGAUGUCCCGGAGCAAGGUGGCUGGGAGCCCG
AGCCCCACACCUGCCGGGCAGCAGGUGCUGGACAUCGACAGCUGCCUGGAC
UCGUCCGUGCUGGACAGCUCCUUCCUCACGUUCUCAGGCCUCCACGCUGAG
CAGGCCUUUGUUGGACAGAUGAAGAGUGACUUGUUUCUGGAUGAUUCUAA
GAGUCUGGUGUGCUGGCCCUCCGGCGAGGGAACGCUCAGUUGGCCGGACC
UGCUCAGUGACCCGUCCAUUGUGGGUAGCAAUCUGCGGCAGCUGGCACGG
GGCCAGGCGGGCCAUGGGCUGGGCCCAGAGGAGGACGGCUUCUCCCUGGCC
AGCCCCUACUCGCCUGCCAAAUCCUUCUCAGCAUCAGAUGAAGACCUGAUC
CAGCAGGUCCUUGCCGAGGGGGUCAGCAGCCCAGCCCCUACCCAAGACACC
CACAUGGAAACGGACCUGCUCAGCAGCCUGUCCAGCACUCCUGGGGAGAA
GACAGAGACGCUGGCGCUGCAGAGGCUGGGGGAGCUGGGGCCACCCAGCC
CAGGCCUGAACUGGGAACAGCCCCAGGCAGCGAGGCUGUCCAGGACAGGA
CUGGUGGAGGGUCUGCGGAAGCGCCUGCUGCCGGCCUGGUGUGCCUCCCU
GGCCCACGGGCUCAGCCUGCUCCUGGUGGCUGUGGCUGUGGCUGUCUCAG
GGUGGGUGGGUGCGAGCUUCCCCCCGGGCGUGAGUGUUGCGUGGCUCCUG
UCCAGCAGCGCCAGCUUCCUGGCCUCAUUCCUCGGCUGGGAGCCACUGAAG
GUCUUGCUGGAAGCCCUGUACUUCUCACUGGUGGCCAAGCGGCUGCACCCG
GAUGAAGAUGACACCCUGGUAGAGAGCCCGGCUGUGACGCCUGUGAGCGC
ACGUGUGCCCCGCGUACGGCCACCCCACGGCUUUGCACUCUUCCUGGCCAA
GGAAGAAGCCCGCAAGGUCAAGAGGCUACAUGGCAUGCUGCGGAGCCUCC
UGGUGUACAUGCUUUUUCUGCUGGUGACCCUGCUGGCCAGCUAUGGGGAU
GCCUCAUGCCAUGGGCACGCCUACCGUCUGCAAAGCGCCAUCAAGCAGGAG
CUGCACAGCCGGGCCUUCCUGGCCAUCACGCGGUCUGAGGAGCUCUGGCCA
UGGAUGGCCCACGUGCUGCUGCCCUACGUCCACGGGAACCAGUCCAGCCCA
GAGCUGGGGCCCCCACGGCUGCGGCAGGUGCGGCUGCAGGAAGCACUCUAC
CCAGACCCUCCCGGCCCCAGGGUCCACACGUGCUCGGCCGCAGGAGGCUUC
AGCACCAGCGAUUACGACGUUGGCUGGGAGAGUCCUCACAAUGGCUCGGG
GACGUGGGCCUAUUCAGCGCCGGAUCUGCUGGGGGCAUGGUCCUGGGGCU
CCUGUGCCGUGUAUGACAGCGGGGGCUACGUGCAGGAGCUGGGCCUGAGC
CUGGAGGAGAGCCGCGACCGGCUGCGCUUCCUGCAGCUGCACAACUGGCUG
GACAACAGGAGCCGCGCUGUGUUCCUGGAGCUCACGCGCUACAGCCCGGCC
GUGGGGCUGCACGCCGCCGUCACGCUGCGCCUCGAGUUCCCGGCGGCCGGC
CGCGCCCUGGCCGCCCUCAGCGUCCGCCCCUUUGCGCUGCGCCGCCUCAGC
GCGGGCCUCUCGCUGCCUCUGCUCACCUCGGUGUGCCUGCUGCUGUUCGCC
GUGCACUUCGCCGUGGCCGAGGCCCGUACUUGGCACAGGGAAGGGCGCUG
GCGCGUGCUGCGGCUCGGAGCCUGGGCGCGGUGGCUGCUGGUGGCGCUGA
CGGCGGCCACGGCACUGGUACGCCUCGCCCAGCUGGGUGCCGCUGACCGCC
AGUGGACCCGUUUCGUGCGCGGCCGCCCGCGCCGCUUCACUAGCUUCGACC
AGGUGGCGCAGCUGAGCUCCGCAGCCCGUGGCCUGGCGGCCUCGCUGCUCU
UCCUGCUUUUGGUCAAGGCUGCCCAGCAGCUACGCUUCGUGCGCCAGUGG
UCCGUCUUUGGCAAGACAUUAUGCCGAGCUCUGCCAGAGCUCCUGGGGGU
CACCUUGGGCCUGGUGGUGCUCGGGGUAGCCUACGCCCAGCUGGCCAUCCU
GCUCGUGUCUUCCUGUGUGGACUCCCUCUGGAGCGUGGCCCAGGCCCUGUU
GGUGCUGUGCCCUGGGACUGGGCUCUCUACCCUGUGUCCUGCCGAGUCCUG
GCACCUGUCACCCCUGCUGUGUGUGGGGCUCUGGGCACUGCGGCUGUGGG
GCGCCCUACGGCUGGGGGCUGUUAUUCUCCGCUGGCGCUACCACGCCUUGC
GUGGAGAGCUGUACCGGCCGGCCUGGGAGCCCCAGGACUACGAGAUGGUG
GAGUUGUUCCUGCGCAGGCUGCGCCUCUGGAUGGGCCUCAGCAAGGUCAA
GGAGUUCCGCCACAAAGUCCGCUUUGAAGGGAUGGAGCCGCUGCCCUCUC
GCUCCUCCAGGGGCUCCAAGGUAUCCCCGGAUGUGCCCCCACCCAGCGCUG
GCUCCGAUGCCUCGCACCCCUCCACCUCCUCCAGCCAGCUGGAUGGGCUGA
GCGUGAGCCUGGGCCGGCUGGGGACAAGGUGUGAGCCUGAGCCCUCCCGCC
UCCAAGCCGUGUUCGAGGCCCUGCUCACCCAGUUUGACCGACUCAACCAGG
CCACAGAGGACGUCUACCAGCUGGAGCAGCAGCUGCACAGCCUGCAAGGCC
GCAGGAGCAGCCGGGCGCCCGCCGGAUCUUCCCGUGGCCCAUCCCCGGGCC
UGCGGCCAGCACUGCCCAGCCGCCUUGCCCGGGCCAGUCGGGGUGUGGACC
UGGCCACUGGCCCCAGCAGGACACCCCUUCGGGCCAAGAACAAGGUCCACC
CCAGCAGCACUUAGUCCUCCUUCCUGGCGGGGGUGGGCCGUGGAGUCGGA
GUGGACACCGCUCAGUAUUACUUUCUGCCGCUGUCAAGGCCGAGGGCCAG
GCAGAAUGGCUGCACGUAGGUUCCCCAGAGAGCAGGCAGGGGCAUCUGUC
UGUCUGUGGGCUUCAGCACUUUAAAGAGGCUGUGUGGCCAACCAGGACCC
AGGGUCCCCUCCCCAGCUCCCUUGGGAAGGACACAGCAGUAUUGGACGGU
UUCUAGCCUCUGAGAUGCUAAUUUAUUUCCCCGAGUCCUCAGGUACAGCG
GGCUGUGCCCGGCCCCACCCCCUGGGCAGAUGUCCCCCACUGCUAAGGCUG
CUGGCUUCAGGGAGGGUUAGCCUGCACCGCCGCCACCCUGCCCCUAAGUUA
UUACCUCUCCAGUUCCUACCGUACUCCCUGCACCGUCUCACUGUGUGUCUC
GUGUCAGUAAUUUAUAUGGUGUUAAAAUGUGUAUAUUUUUGUAUGUCACU
AUUUUCACUAGGGCUGAGGGGCCUGCGCCCAGAGCUGGCCUCCCCCAACAC
CUGCUGCGCUUGGUAGGUGUGGUGGCGUUAUGGCAGCCCGGCUGCUGCUU
GGAUGCGAGCUUGGCCUUGGGCCGGUGCUGGGGGCACAGCUGUCUGCCAG
GCACUCUCAUCACCCCAGAGGCCUUGUCAUCCUCCCUUGCCCCAGGCCAGG
UAGCAAGAGAGCAGCGCCCAGGCCUGCUGGCAUCAGGUCUGGGCAAGUAG
CAGGACUAGGCAUGUCAGAGGACCCCAGGGUGGUUAGAGGAAAAGACUCC
UCCUGGGGGCUGGCUCCCAGGGUGGAGGAAGGUGACUGUGUGUGUGUGUG
UGUGCGCGCGCGCACGCGCGAGUGUGCUGUAUGGCCCAGGCAGCCUCAAG
GCCCUCGGAGCUGGCUGUGCCUGCUUCUGUGUACCACUUCUGUGGGCAUG
GCCGCUUCUAGAGCCUCGACACCCCCCCAACCCCCGCACCAAGCAGACAAA
GUCAAUAAAAGAGCUGUCUGACUGCAA

In some embodiments, PKD1 refers to human PKD1 having a 5′UTR having the RNA sequence as set forth in SEQ ID NO:2.

Human PKD1 5′UTR (SEQ ID NO: 2)

GCACUGCAGCGCCAGCGUCCGAGCGGGCGGCCGAGCUCCCGGAGCGGCCUG
GCCCCGAGCCCCGAGCGGGCGUCGCUCAGCAGCAGGUCGCGGCCGCAGCCC
CAUCCAGCCCCGCGCCCGCCAUGCCGUCCGCGGGCCCCGCCUGAGCUGCGG
CCUCCGCGCGCGGGCGGGCCUGGGGACGGCGGGGCCAUGCGCGCGCUGCCC
UAACG

In some embodiments, PKD1 refers to human PKD1 having the following upstream open reading frames in the 5′UTR thereof:

Human PKD1 5′UTR uORF1
(SEQ ID NO: 3, initiation codon highlighted)
AUGCCGUCCGCGGGCCCCGCCUGA
Human PKD1 5′UTR uORF2
(SEQ ID NO: 4, initiation codon highlighted)
AUGCGCGCGCUGCCCUAA
Human PKD1 5′UTR uORF3
(SEQ ID NO: 5, initiation codon highlighted)
CUGGCCCCGAGCCCCGAGCGGGCGUCGCUCAGCAGCAGGUCGCGGCCGCAG
CCCCAUCCAGCCCCGCGCCCGCCAUGCCGUCCGCGGGCCCCGCCUGAGCUG
CGGCCUCCGCGCGCGGGCGGGCCUGGGGACGGCGGGGCCAUGCGCGCGCUG
CCCUAA
Human PKDI 5′UTR uORF4
(SEQ ID NO: 6, initiation codon highlighted)
CUGCAGCGCCAGCGUCCGAGCGGGCGGCCGAGCUCCCGGAGCGGCCUGGCC
CCGAGCCCCGAGCGGGCGUCGCUCAGCAGCAGGUCGCGGCCGCAGCCCCAU
CCAGCCCCGCGCCCGCCAUGCCGUCCGCGGGCCCCGCCUGAGCUGCGGCCU
CCGCGCGCGGGCGGGCCUGGGGACGGCGGGGCCAUGCGCGCGCUGCCCUAA

In some embodiments, Pkd1 refers to mouse Pkd1 having the following mRNA sequence:

Mus musculus polycystin 1, transient receptor potential channel
interacting (Pkd1), mRNA (SEQ ID NO: 7)

UCCAGUCCCACCUACAGCCAACUUGGAAGCGCAGGGGCGGAGUCUGAGGG
CGCCCCGCCCCACGCCGGAGGUGGGGGAGGAGGAGGAGCUGGCGCUGGGU
CUGCAGUGCAGCGCGAAUGCGCGAGCAGGCGGCCAAGGCCCUGAGGUGCG
GCCUGCCCCAGAGCGCUGAGCAGCUGUCGCACCGCAGACGGGCCACGGCCG
CCGCGGCAUUCAGCCCCGUGCCGCACCAUGAGGUCCGCGGGCCCCUCCUGA
ACUGCGGCUGCCGGCGCAAGGGGCCUCGGGGAACCGGGGCCAUGCGCGGGC
UGCGCUGACGAUGCCGCUCGGCGCGCCUGCUCUCCUGGCGCUGGCCCUAGG
CCUGGGCCUGUGGCUCGGAGCGCUGGCCGGAGACCCUGGGCGCGGCUGCGG
GCCUUGCCCGCUCCCCUGCUUUUGCGGCCCUGCGCCCGACGCCGCCUGCCG
CGUCAAUUGCUCCGGCCGCUGGCUGCAGACGCUAGGGCCGAGUCUGCGCAU
CCCGGCUGACGCCACCGCGCUAGACCUGUCCCACAACCUACUUCAGACGCU
GGACAUAGGGCUCCUGGUGAACCUUUCAGCACUGGUGGAGCUGGACUUAA
GCAACAAUAGGAUUUCUACAUUAGAAGAAGGCGUGUUUGCUAAUUUAUUU
AAUUUAAGUGAAAUAAACCUGAGUGGGAACCCAUUUGAGUGCAACUGUGG
UCUGGCGUGGCUGCCACGCUGGGCAAAGGAACAUCAGGUGCAUGUGGUAC
AGUCUGAGGCAACCACGUGCAGGGGGCCCAUUCCACUGGCCGGCCAACCUC
UCCUCAGUAUCCCCUUAUUGGACAAUGCCUGUGGUGAGGAAUAUGUCGCC
UGCCUCCCUGACAACAGCUCAGGUGCUGUGGCAGCAGUACCCUUCUACUUU
GCCCAUGAGGGACCACUGGAGACUGAGGCCUGCAGUGCCUUCUGCUUCUC
AGCUGGUGAGGGCCUUGCAGCCCUCUCGGAGCAGAAUCAAUGCCUCUGUG
GGGCAGGCCAGGCCUCUAACUCUUCUGCAGCCUGCUCAUCCUGGUGUUCCA
GCAUCUCACUGUCCCUCAACUCUGCCUGUGGGGGCCCCACCUUGCUCCAGC
ACACCUUUCCCGCCUCCCCAGGGGCUACCCUGGUGGGGCCCCAUGGACCCC
UGGCCUCUGGCCAGCCAGCAGACUUCCAUAUUACUUCCUCCCUGCCCAUUA
GCUCAACACGCUGGAACUUUGGAGAUGGCUCACCUGAGGUGGACAUGGCU
AGCCCUGCUGCCACCCACUUCUAUGUGCUACCUGGGAGCUAUCACAUGACA
GUUGUGCUGGCCUUGGGGGCUGGCUCAGCUCUGCUGGAGACAGAGGUGCA
AGUAGAAGCAACACCUACUGUCCUGGAGCUUGUGUGCCCAUCCUUUGUGC
ACAGUAACGAGAGCCUUGAACUUGGCAUACGGCAUCGUGGAGGCUCAGCC
CUUGAGGUCACCUAUAGCAUCCUGGCUUUGGAUAAAGAGCCUGCCCAAGU
GGUCCACCCACUCUGCCCCUUGGACACAGAGAUCUUCCCUGGCAAUGGGCA
CUGCUACCGCCUGGUGGCAGAGAAGGCACCCUGGCUGCAAGCACAGGAGC
AAUGUCGGACUUGGGCUGGAGCUGCCUUGGCUAUGGUGGACAGUCCUGCC
AUCCAGCACUUCCUAGUCUCCAAGGUUACCAGGAGCCUGGAUGUGUGGAU
CGGCUUCUCAUCUGUGGAGGGGACAGAAGGCCUGGAUCCCCGGGGUGAGG
CCUUCAGCCUGGAAAGCUGCCAGAACUGGCUGCCUGGGGAGCCACACCCAG
CCACAGCAGAGCAUUGUGUGCGGUUGGGGCCUGCUGGGCAAUGCAACACA
GACCUGUGUUCAGCACCACAUAGUUAUGUCUGCGAGCUGCGGCCUGGAGG
GCCUGUGUGGGAUACUGAGAACUUUGUCAUGGGAAUGUCUGGUGGAGGCC
UGUCUGGACCUCUGCAUCCAUUGGCACAGCAGGAAACUGUCCAGGGCCCGC
UGCGACCUGUGGAGGUCAUGGUGUUCCCUGGCCUGAGCCCUAGCCGGGAA
GCCUUUCUCACUGCAGCAGAGUUUUCUACCCAGAAGCUUGAAGAGCCUGC
CCAGAUGCGGCUGCAGGUGUACCGGCCCUCAGGAGGAGCAGCAGCGGUCCC
AGAAGGCAGCAGUGAGCCUGACAACAGGACUGAGCCGGCCCCCAAGUGUG
UGCCUGAGGAACUCUGGUGUCCAGGAGCCAAUGUCUGCAUACCUUUUGAU
GCUUCGUGUAACUCUCAUGUCUGCAUCAAUGGGUCUGUGUCAAGGCUGGG
ACUCUCUAGGGCCAGCUACACACUAUGGAAGGAGUUCUUUUUUUCUGUGC
CUGCAGGGCCUCCCACACAGUACUUGGUCACCCUCCACAGCCAGGAUGUUC
CUAUGCUCCCUGGUGACCUCAUUGGCUUACAGCAUGAUGCUGGCCCAGGU
ACUCUCCUACAGUGUCCCCUGGCUUCCAGCUGUCCUGGCCAAGCCCUGUAC
CUUUCAACCAAUGCCUCAGAUUGGAUGACCAACCUGCCUGUCCAUCUGGA
AGAAGCUUGGGCUGGCCCUGUCUGCUCCCUACAGCUGCUUUUGGUCACAG
AGCGGCUUACACCACUGCUGGGCCUUGGGCCUAACCCUGGACUACAGCAUC
CUGGGCACUAUGAGGUCCGGGCUACAGUGGGCAACAGCGUAUCCAGGCAA
AACCUGUCCUGCAGCUUCAGUGUGGUCUCUCCAAUAGCUGGGCUGAGAGU
CAUUCACCCCAUCCCCCUUGAUGGCCACAUCUAUGUGCCAACCAACGGCUC
AGUCUUGGUGCUUCAGGUAGAUUCUGGUGCUAAUGCCACUGCCACAGCUC
AGUGGUUUGGGGGCAAUAUUAGUGCUCCCUUCGAGGAUGCCUGCCCUCCU
GAGGUGGAUUUUCUCAAGCAAGAUUGCACAGAAGAGGCCAAUGGCACCCU
GUUCUCAGUGUUGAUGUUGCCCAGGCUCAAGGAGGGGGAUCACACAGUGG
AGAUUGUGGCGCAAAAUGGAGCCAGCCAGGCCAACCUCAGCCUGAGAGUG
ACAGCUGAGGAGCCCAUCUGUGGCCUCCGUGCUGUGCCAAGCCCUGAGGCC
CGAGUGCUACAGGGCAUCCUGGUGCGGUACAGCCCCAUGGUAGAGGCUGG
CUCAGACGUAGCCUUCCGGUGGACCAUUGAUGAUAAGCAGUCUCUGACUU
UCCACAACACUGUCUUCAAUGUAAUAUACCAGAGUGCUGCCAUCUUCAAG
CUCUCGCUGACAGCCUCUAACCAUGUGAGCAACAUCACCGUGAACUACAAU
GUCACUGUGGAGCGGAUGAACAAGAUGCAUGGGCUGUGGGUAUCUGCAGU
ACCGACUGUGUUACCCCCCAAUGCCACACUGGCACUGACAGGUGGUGUGCU
GGUGGAUUCAGCUGUGGAGGUGGCCUUCCUGUGGAACUUUGGGGAUGGGG
AGCAGGUACUCCGCCAGUUCAAGCCUCCAUACGACGAGUCCUUCCAGGUUC
CAGACCCCACAGUGGCGCAGGUGCUGGUGGAACACAAUACCACGCAUAUU
UACACUACCCCAGGUGAGUACAACUUGACCGUCCUUGUGUCCAAUACCUA
UGAGAAUCUGACGCAGCAGGUGACAGUAAGUGUGCGCACUGUUCUUCCCA
ACGUGGCUAUUGGCAUGAGCAGCAACGUCCUGGUGGCCGGCCAACCCAUC
ACCUUUUCCCCAUAUCCGCUGCCCUCAACUGAUGGUGUCCUAUAUACGUGG
GACUUUGGGGAUGGCUCACCUGUCCUAAUACAGAGCCAGCCAGUACUCAA
CCACACCUACAGCAUGACGGGCGCUUACAGAAUCACCUUGGAGGUUAACA
ACACAGUGAGCAGCGUGACAGCACAUGCAGACAUCCGUGUCUUCCAGGAG
UUGCAUGGCCUGACUGUGUAUUUGAGCCCAUCUGUGGAGCAGGGAGCCCC
CAUGGUAGUCAGUGCUUCUGUGGAGUCAGGUGACAACAUUACAUGGACAU
UUGACAUGGGAGAUGGCACAGUAUUCACAGGCCCUGAGGCUACAGUACAA
CAUGUAUACCUGCGGGCCCAGAACUUCACAGUGACAGUAGAAGCAGCCAA
CCCUGCUGGCCACUUGUCCCAGAGCCUGCAUGUGCAAGUGUUUGUCCUGG
AGGUAUUACACAUAGAGCCCUCUACCUGCAUCCCCACACAGCCCAGUGCCC
AGCUUAUGGCCCAUGUCACUGGGGACCCUGUCCAUUACCUCUUUGAUUGG
ACUUUUGGAGAUGGCUCCUCCAAUGUGACUGUCCAUGGGCAUCCAUCAGU
GACACACAACUUCACUAGGAGUGGCAUAUUCCCACUGGCAUUGGUUUUGU
CAAGCCAUGUGAACAAGGCACAUUAUUUUACCAGCAUCUGUGUGGAACCU
GAGAUACGUAACAUCACCCUGCAGCCUGAGAGGCAGUUUGUGAAGCUGGG
GGAUGAAGCCCGGCUGGUUGCCUAUUCUUGGCCCCCAUUCCCGUACCGAUA
UACCUGGGACUUUGGCACUGAAGACACUACCCACACCCAAACUGGGGGCUC
UGAAGUGAAAUUUAUCUACCGAGAACCAGGCUCCUACUUGGUGAUAGUCA
CUGUGUCCAACAACAUCUCUUCUACCAAUGACUCAGCUUUUGUGGAGGUC
CAGGAGCCUGUGUUAGUCACUGGCAUCAGGAUCAAUGGCUCCCAUGUGUU
AGAGCUGCAGCAGCCUUACCUGUUGUCAGCCAUGGGCAGUGGGAGCCCUG
CCACCUACCUAUGGGAACUGGGAGACGGCAGCCAGAGUGAGGGCCCUGAG
GUCACCCAUAUCUACAGCAGCACCGGUGAUUUCACUGUCAGGGUGUCUGG
CUGGAAUGAGGUGAGCCGUAGUGAGGCCCAACUCAACAUCACCGUAAAGC
AGCGUGUGCGGGGCCUCACCAUCAAUGCCAGCCGCACAGUGGUGCCCCUGA
AUGGCAGUGUGAGCUUUAGUACCUUGUUGGAGGUAGGGAGUGAUGUGCAU
UACUCCUGGGUGCUCUGUGACAGAUGCACACCCAUUCCAGGCGGACCCACU
AUUUCCUACACCUUCCGCUCUGUGGGCACCUUCAAUAUUAUUGUCACAGC
UGAGAAUGAGGUGGGUUCUGCCCAGGACAGCAUCUUCAUCUAUGUUCUCC
AGUUCAUUGAGGGGCUGCAGGUGGCAGGGGGUGAUAACGGCUGCUGCUUC
CCUACCAACUACACACUGCAGCUGCAAGCUGCAGUUAGGGAUGGCACCAAC
AUCUCUUAUAGCUGGACAGCGCAGCAGGAGGGCAGCCUCAUCACACUCUU
CGGCAGUGGCAAGUGCUUUUCUCUCACUUCACUCAAAGCUAGCACCUACU
AUGUCCAUCUUAGGGCCACUAACAUGUUGGGUAGUGCCGCAGCCAACCGU
ACUAUAGACUUUGUGGAACCUGUGGAGAGUCUAAUCCUAUCUGCAUCCCC
UAAUCCAGCUGCUGUCAACAUGAGUCUCACCCUUUGUGCUGAAUUGGCUG
GUGGCAGUGGUGUUGUUUACACUUGGUACCUGGAGGAAGGACUGAGUUGG
AAGACCUCCAUGCCAUCUACCACCCACACCUUUGCUGCACCUGGCCUGCAC
UUGGUCAGAGUCACAGCUGAGAACCAGUUGGGCUCAGUCAAUGCCACAGU
UGAAGUGGCCAUACAGGUGCCUGUGGGUGGUCUGAGUAUCAGAACCAGUG
AGCCAGAUAGCAUCUUUGUGGCAGCUGGCUCCACUCUACCCUUCUGGGGU
CAGCUGGCUGAAGGUACCAAUGUGACUUGGUGCUGGACCUUGCCAGGUGG
GAGUAAGGACAGCCAGUACAUUGCUGUGCGCUUCUCCACGGCUGGCAGUU
UCUCUCUGCAGCUCAAUGCUUCCAAUGCAGUCAGUUGGGUCUCAGCCAUG
UACAACCUCACAGUAGAGGAACCCAUUGUGAACCUGAUGCUGUGGGCCAG
CAGCAAGGUGGUGGCGCCUGGGCAGCCAGUCCACUUUGAAAUCCUGCUGG
CAGCUGGCUCUGCUCUUACUUUCCGACUUCAGGUUGGUGGGUCUGUCCCU
GAAGUGCUCCCUAGCCCCCACUUCUCCCACAGCUUCUUUCGGGUUGGAGAC
CAUCUGGUGAAUGUGCAGGCUGAGAACCACGUGAGUCAUGCCCAGGCACA
GGUUCGCAUCCUAGUGCUGGAGGCUGUUGUUGGGCUGCAGGUACCCAACU
GCUGUGAGCCAGGCAUGGCCACAGGCACUGAGAAGAACUUCACAGCCCGG
GUGCAGCGAGGUUCCCGAGUUGCCUAUGCCUGGUAUUUCUCCUUGCAAAA
GGUUCAGGGUGACUCAUUGGUUAUUUUAUCUGGCCGUGAUGUCACCUACA
CACCUGUGGCUGCAGGGUUACUGGAGAUCCAUGUGCGUGCCUUUAAUGAA
CUGGGUGGUGUGAACCUCACACUCAUGGUGGAAGUCCAGGAUAUCAUCCA
GUAUGUAACACUUCAGAGUGGCCGCUGCUUUACCAACCGCUCAGCCAGGU
UUGAGGCUGCUACAAGCCCCAGUCCACGGCGUGUAACCUACCACUGGGACU
UUGGGGAUGGGACUCCGGUACAGAAAACAGAGGAAUUCUGGGCUGAUCAC
UACUACCUGCGUCCUGGGGACUACCAUGUAGAGGUGAAUGCUACAAACCU
AGUGAGUUUUUUUGUGGCACAGGCCACAGUGACUGUCCAGGUGCUGGCCU
GUAGGGAGCCUGAAGUGGAGGUAGCACUGCCUCUGCAGGUGCUGAUGCGG
CGUUCCCAGCGCAACUACCUGGAGGCCCAUGUUGAUCUACGCAACUGUGUC
UCCUACCAGACUGAAUAUCGGUGGGAGAUAUAUCGUACUGCCAGCUGCCA
ACGGCCAGGACGUAUGGCUCAAAUGGUACUGCCUGGUGUGGAUGUGAGCC
GACCCCAGCUGGUGGUGCCACGGCUGGCACUACCUGUGGGCCACUACUGCU
UUGUGUUUGUGGUUUCAUUUGGGGACACACCAUUGGCACGGAGCAUCCAG
GCCAAUGUGACAGUGGCUGCUGAGCGUCUGGUACCCAUCAUUGAAGGUGG
CUCAUACCGGGUAUGGUCAGACACGCAGGACCUGGUGCUGGAUGGGAGCA
AGUCCUAUGACCCUAAUUUAGAGGAUGGUGAUCAGACACCGCUCAACUUC
CAUUGGGCCUGCGUGGCUUCAACACAGAGUGAGACAGGUGGCUGCGUGCU
AAACUUUGGGCCCCGUGGGAGCAGUGUGGUCACCAUCCCCCUGGAACGCCU
AGAAGCUGGUGUAGAAUAUACCUUCAACCUCAUAGUGUGGAAAGCAGGUC
GGAAGGAAGAAGCCACCAACCAGACAGUACUGAUCCGAAGUGGGCGAGUC
CCCAUUGUGUCCCUUGAGUGUGUGUCCUGCAAGGCACAAGCAGUGUAUGA
AGUGAGCCGAAGUUCCUAUGUGUACUUGGAGGGUCACUGCCACAACUGCA
GCAGAGGCUACAAGCAAGGGUGUUGGGCCGCUCGCACUUUCAGCAAUAAG
ACUCUGGUGCUGAACGAGACGACAACAUCCACGGGUAGCACCGGCAUGAA
UCUGGUAGUGCGGCCAGGUGCACUGCGUGAUGGGGAGGGCUACAUCUUCA
CACUCACUGUGCUGGGCCACUCAGGGGAAGAAGAGGGCUGUGCCUCAAUC
CGCCUCUCACCCAACCGCCCCCCACUUGGAGGCUCCUGCCGCCUAUUCCCA
UUGGAUUCUGUGCGUGGCCUCACCACUAAGGUUCACUUCGAGUGCACAGG
UUGGCGUGAUGCAGAAGAUGGUGGAGCCCCACUGGUAUAUGCUCUGUUGC
UGAAGCGCUGUCGUCAGAGCUACUGUGAGAAUUUCUGCAUCUAUAAGGGU
AGUCUUUCCACCUAUGGGGCUGUCCUGCCUCCUGGAUUCCAGCCCCUUUUU
GUGGUGAGCCUAGCUGUGGUUGUACAAGACCAGCUGGGUGCUGCUGUUGU
UGCACUCAACAGGUCUCUGACCAUUGUCCUACCAGAACCCAGUGGAAAUCC
UGCAGACCUCGUACCCUGGCUGCACAGCCUGACUGCCAGUGUGCUGCCUGG
GCUCCUGAAGCAGGCUGACCCUCAGCAUGUCAUCGAGUACUCUUUAGCCCU
CAUUACUGUGCUCAAUGAGUAUGAGCAGGCCCCAGAUGUGUCAGAGCCCA
ACGUUGAGCAGCAGCUGAGAGCCCAAAUGCGUAAGAACAUCACGGAGACC
CUGAUCUCCCUGCGGGUCAACACUGUGGAUGACAUCCAGCAGAUUACUGC
UGCGUUAGCCCAGUGCAUGGUGUCCAGCAGGGAGCUCAUGUGCCGCUCCU
GCCUGAAGAAGAUGCUGCAGAAGCUGGAGGGCAUGAUGCGCAUCUUGCAA
GCUGAAACCACUGAGGGCACCUUGACACCCACCACCAUUGCUGACAGCAUU
CUCAAUAUCACAGGUGACCUCAUCCACCUGGCCAGCUUGGACAUGCAGGGC
CCACAGCCCUUGGAGCUGGGGGUGGAGCCGCCCUCCCUGAUGGUGGCUUCU
AAGGCCUACAACCUCUCGUCGGCCCUCAUGCGUAUCCUCAUGCGUUCCCGA
GUGCUCAAUGAGGAGCCUCUGACUCUGGCUGGCGAGGAGAUUGUGGCCCU
AGGCAAGCGCUCAGACCCACUCAGUCUUCUGUGUUAUGGCAAAGCCUUGG
GGCCCAGCUGCCACUUCUCCAUUCCUGAGGCCUUCAGUGGGGCCCUGUCGA
ACCUCAGUGAUGUGGUGCAGCUCAUCUUCCUUGUGGAUUCCAACCCCUUCC
CCUUUGGCUACAUUAGCAACUACACUGUCUCCACCAAAGUGGCAUCUAUG
GCAUUCCAGACACAGACUGGUACCCAGAUCCCUAUUGAGCAGUUGGCUGC
AGAGCGUGCCAUCACAGUGAAGGUGCCAAACAACUCAGACCAGGCUGCCC
AGAGCUCUCACAAUCCUGUGGGUUCUACCAUUGUACAGCCCCAGACCUCUG
UCAGCGCUGUGGUCACUGCAGACAACAGUAACCCUCAAGCUGGCCUGCAUC
UGAGGAUCACUUACACAGUGCUUAAUGAGCGCUAUCUGUCCGCGGAGCCU
GAGCCCUACCUGGCUGUGUACCUGCACUCAGUGUCCCAGCCCAAUGAGUAC
AACUGCUCGGCCAGCAGGAGGAUCAGCCUGGAGGUGCUGGAGGGUGCUGA
CCACAGGCUCUACACCUUCUUCAUUGCCCCAGGAACUGGGACCUUGGACAG
GAGUUACUACCUGAACCUGACGAGCCAUUUCCACUGGUCUGCCCUGGAGG
UGUCUGUGGGCCUCUAUACAUCCCUGUGCCAGUACUUCAGUGAGGAGAUG
AUGAUGUGGAGAACUGAGGGCAUUGUACCCCUGGAGGAGACCUCACCCAG
CCAGGCUGUCUGCCUCACCCGCCACCUCACUGCCUUCGGUGCCAGCCUUUU
UGUGCCUCCCAGCCAUGUCCAGUUCAUCUUUCCUGAACCAUCUGCAAGCAU
CAACUACAUUGUCCUCCUGACGUGUGUCAUAUGCCUGGUGACCUAUGUGG
UCAUGGCUAUGAUCCUGCGCAAGCUGGACCAGCUGGAUGUUAGCCGUGUC
CGUGUCAUCCCGUUCUGUGGGAAGGGGGGCCGCUUCAAAUAUGAAAUACU
UGUUAAGACCGGCUGGAGCCGAGGUUCAGGUACCACUGCUCACGUCGGCA
UCAUGCUGUAUGGAGAGGACAACCGUAGUGGUCACCGGCACUUAGAUGGG
GACAGAGCCUUUCAUCGAAACAGCCUGGACAUCUUCCAAAUUGCCACCCCG
CACAGCCUGGGCAGCGUGUGGAAGAUCCGAGUGUGGCAUGAUAACAAAGG
GCUCAGCCCUGCCUGGUUCCUGCAACACAUUAUCGUCCGGGACCUACAGAG
UGCCCGCAGCACCUUCUUUUUGGUCAAUGACUGGCUGUCUGUGGAGACUG
AGGCCAACGGGGGCCUAGUGGAGAAGGAGGUGCUUGCAGCAAAUGAAGCU
GCGCUGUGGCAGUUCCAGCGGCUCCUCGUGGCUGAGUUGCAGAGAGGCUU
CUUUGACAAGCACAUCUGGCUCUCCAUAUGGGACCGGCCGCCUCGUAGCCG
CUUUACUAGAGUCCAGAGGGUUACCUGCUGUGUUCUCCUCCUCUGCCUCU
UCCUGGCUGCCAAUGCUGUGUGGUACGGAGUUGUGAGAGACACCACCUAC
AGCAUGGGACCUGUGUCCAGUCUGAUCUCUCCUGGUGUUGACACAGUUGC
UAUUGGCCUUGUGUCCAGUGUGGUAGUCUACCCUGUCUAUUUGGCUGUCC
UGUUCCUCUUCCGGAUGUCUCGGAGUAAGGUGUCUGGGGACCAAAACCCC
ACACCCACAGGGCAGCAGGCCCUAGAUGUUGACAGCUACCUGGACCCAUCU
GUGCUAGACAGCUCCCUCCUCACACUCUCUGGCCUCACAGAGGCCUUCGCU
GGGCAAGUGAAGAAUGACUUGUUUCUGGAAGAUGCUAAAAGCCUGGUGUG
CUGGCCGUCCAGUGAGGGUACUCUUAGUUGGCCAGACCUGCUCAGUGACC
CAUCUGUUGUGAGCAGCACACUGCAGCGAUUGACCCAGGGCAGGCCUGGC
UGCAUGUUGGGCUCAGAGGAAGAUGGUGCCUCUCUGGUUAGCCCUUCCUU
GCCUGCCAAAUACUUGUCGGCCUCAGAUGAAGACCUGAUCCAUCAGGUAC
UGGCUGAUGGGGCCAACAACCUGGUUCCUACCCAGGACACCCUCCUAGAAA
CAGACCUGCUUACUAGCCUGUCCAGUGUUCCUGGAGAGAAAACAGAAACU
CUGAUACUGCAGACAGUGGGGGAGGAGAGACCAGCCAGCAUGGGCCUCAG
UUGGGAACAGUCCCCGGUGACAAGGCUUUCCAGGACAGGACUGGUGGAAG
GUUUUCAGAAGCGACUGCUGCCUGCCUGGUGUGCCCCACUGGCCCAUGGGC
UCAGCCUGCUCUUGGUAGCUGUAGCUGUAGCAGUUUCAGGGUGGAUUGGU
GCCAGCUUCCCUCCCAGCGUGAGUGUCAUGUGGCUUCUCUCAAGCAGCUCC
AGCUUCCUGGCCUCCUUUCUUGGCUGGGAGCCCCUUAAGGUCCUGCUGGA
AGCCCUCUACUUCUCCCUUGUGGCCAAGCGGCUUCACCCUGAUGAAGAUGA
CACUCUGGUAGAGAGCCCAGCUGUAACCCCUGUAAGUGAGCGUGUGCCCC
GUGUGCGGCCCCCACAUGGUUUUGCACUCUUCUUGGCAAAGGAGGAAGCU
CGAAAGGUCAAGAGGCUCCAUGACAUGUUGAAGAGACUCUUGGUGUACAU
GCUUUUCUUACUGGUGACGCUGCUGGCCAACUAUGGGGAUGCUUCUUGCC
AUGGUCACGCCUAUCGCCUGCAGAGCGCCAUCAAGCAGGAGCUGGAUAGC
CAAGCCUUUCUGGCCAUCACCCGGUCUGAUGAGUUCUGGCCAUGGAUGUC
ACAUGUGUUCUUGCCCUAUGUCCAUGGGAACCAGUCUAGCCCUGAGCUGG
GCCCUCCACGGCUACGGCAGGUGCGACUGCAGGAAGCAUUCUGCCCAGACC
CUUCUAGCUCGGAGCACAUGUGUUCAGCGGCAGGAAGCUUGAGCACCAGU
GACUAUGGCAUUGGCUGGCAGAGUGUGGUGCAAAAUGGCUCUGAGACAUG
GGCCUACUCUGCACCGGACCUGCUGGGCGCCUGGUACUGGGGCUACUGUGC
AGUGUAUGACAGUGGGGGCUACAUACAGGAGUUAGGACUGAGUCUGGAGG
AGAGCCGUGCACGACUGGGCUUCCUGCAGCUGCACAACUGGCUUGACAGC
AGGAGCCGUGCAGUGUUUGUAGAACUCACCCGCUACAGCCCUGCUGUGGG
GCUCCAUGCUGCUGUUACACUGCGCCUCGAGUUCCCUGUAGCCGGCCAUGC
ACUGGCUGCCUUCAGUGUCCGCCCAUUUGCGUUACGGCGACUCAGCACUGG
CCUGUCUCUACCACUGCUCACCUCGGUGUGCUUGCUGCUGUUUGCCCUAUA
CUUUUCCAUGGCCGAGGUGCAGACCUGGCGCAAAGAUGGGUGUGCGUGCA
CAGCAAGGCCUGACACCUGGGCACGGUGCCUACUGGUGAUACUGACAGCA
GCUACAGGGCUUGUACGCCUGGCCCAGCUGGGCAUUGCUGAUCGCCAGUG
GACCCAUUUUGUGCAGGACCAUCCACGCCACUUCACUAGCUUCGACCAAGU
GGCUCAGCUGGGCUCUGUGGCCCGUGGCCUGGCAGCUUCACUACUCUUCCU
GCUUUUGGUCAAGGCUGCCCAGCAGCUGCGGUUUGUGCGCCAGUGGUCUG
UUUUUGGCAAGACAUUGUGUCGGGCCUUGCCCGAGCUCAUGGGAGCCACC
UUGGGCCUGGUGCUGCUUGGUGUGGCCUAUGCACAGAUGGCCAUCCUGCU
UAUUUCCUCUGGUGCUGACACUCUCUACAACAUGGCCCGUGCCUUCCUGGU
GCUGUGCCCUGGGGCGAGGGUCCCCACUCUGUGCCCUUCAGAGUCCUGGUA
CCUGUCCCCUCUGCUGUGUGUGGGGCUCUGGGCUCUGCGAGUGUGGGGUG
CCCUGAGGUUAGGGGCUAUCCUCCUGCGUUGGCGGUACCACGCCUUGCGU
GGCGAGCUCUACCGCCCAGCAUGGGAACCCCAGGACUAUGAGAUGGUGGA
GCUUUUCCUGCGUAGGCUUCGGCUCUGGAUGGGCUUCAGCAAGGUCAAGG
AGUUCCGCCACAAAGUCCGCUUUGAAGGAAUGGAUCCACUGCCUUCCCGCU
CAUCCAGGGGCUCCAAGUCAUCCCCAGUUGUGCUCCCACCUAGCUCAGGCU
CAGAAGCUUCACACCCAUCCACCUCGUCCAGCCAACCAGACGGGCCGAGCG
CCAGCUUAAGCCGCUCGACGCUGAAGCUGGAACCAGAGCCCUCUCGCCUCC
AUGCUGUGUUUGAAAGUCUGCUUGUCCAGUUUGACCGACUCAACCAGGCC
ACAGAGGACGUCUACCAGCUGGAGCAACAACUCCAGAGCCUUCAAGGCCA
UGGGCACAAUGGACCUCCUUCCUCUCCCUCCCCUGGCUGCUUCCCAGGCUC
UCAGCCAGCUUUGCCCAGCCGCCUUUCUCGGGCCAGUCAGGGGCUGGAUCA
GACUGUAGGCCCCAACAGGGUGUCCCUGUGGCCUAAUAACAAGGUCCACCC
CAGCAGCACUUAGGCCCUAGGGGUCUUGGCCAUUCCCUUCCCUGGGAAUGC
CUGAGCUUCACACUGGCCUCUCAGAGCCAGGGUGGACACCACUCAGUAUU
ACCUUCUGCUGCCCUCUAGGUUGGGCCAGGCAGAACGGCUGCAUGCCAGU
UCUUUUGGGUACAGGUAUUGCUGCCUUCCUUACCUGUCCACAUAUGGGGC
UUCUGCACUUUAAAAAGGCUGUGUGGCCAGCCAGGACCCAGGGUCCCCUCC
CCACAGGAGGACACAGCAGUAUUGGACCAAGUGAGUACCCAGCCUCCAAG
AUGCUAAUUUAUUCGCCCCCCCUCCCCCCAAGUCCUCAGGUUCAGCGGGCU
GCGCCCAGCUCUACCCCUAGGUGGCCAUCUCCUCUUGCUAAAAAUCUAAGC
UGGAGGGAGGGCUAAGCUACACUUCUCCUUUAUCCGUCCCCUAAGUUAUU
ACCUCUCUUGUUUCUGCAGCAUACUUGCCCUCUAGCUACCAUCUGCUUUCU
AUGUCCACCAUCAAUAAUUUAUAUGGGGUUAAAAUGUAUAUAUUUUUGUA
UGUCAGUAUUUUCUACUUGGGCUGAAAAUGGGUCCAUUGUUACCCAGGGC
AGGGGUACAGGGGAAGCUGCAGCUAUUACUGGAUCCGAGUCUGGCCUCGG
GCAGGUGCUGGACAGCAAUUGGGGCCACCGUGGCCUUGUUUUCCUCCCUU
GUCUGGGGCCAGGCAGCACGCAGAUCUGCUGGUUUCAGAUCUGAGCAAGG
GCAGGACUACUGUGGGAUGAAAGAUGCCAAGAAGUUACUGGACAAGCCCA
CCAAUGGGGCUUGCCCCCAUGGCGGGUGGGGGAAAGAGAAUGUAUGUGAU
GGCACCUGCCUGCUUCUGUGGGCUCAUCUGCAGCUCUAGCCUGGAUUCUGC
CCCAACCCCAAACAGACAAGACAAAGUCAAAUAAAGAAGCUGUCUGACUG
C

In some embodiments, Pkd1 refers to mouse Pkd1 having a 5′UTR having the RNA sequence as set forth in SEQ ID NO:8.

Mouse Pkd1 5′-UTR (SEQ ID NO: 8)

UCCAGUCCCACCUACAGCCAACUUGGAAGCGCAGGGGCGGAGUCUGAGGG
CGCCCCGCCCCACGCCGGAGGUGGGGGAGGAGGAGGAGCUGGCGCUGGGU
CUGCAGUGCAGCGCGAAUGCGCGAGCAGGCGGCCAAGGCCCUGAGGUGCG
GCCUGCCCCAGAGCGCUGAGCAGCUGUCGCACCGCAGACGGGCCACGGCC
GCCGCGGCAUUCAGCCCCGUGCCGCACCAUGAGGUCCGCGGGCCCCUCCU
GAACUGCGGCUGCCGGCGCAAGGGGCCUCGGGGAACCGGGGCCAUGCGCG
GGCUGCGCUGACG

In some embodiments, Pkd1 refers to mouse Pkd1 having the following upstream open reading frames in the 5′UTR thereof:

Mouse Pkd1 5′UTR uORF1
(SEQ ID NO: 9, initiation codon highlighted)
AUGAGGUCCGCGGGCCCCUCCUGA
Mouse Pkd1 5′UTR uORF2
(SEQ ID NO: 10, initiation codon highlighted)
AUGCGCGGGCUGCGCUGA

For the purposes of the instant specification, the terms “first uORF,” “second uORF,” “third uORF” or “fourth uORF” does not mean these uORFs are located in the mRNA sequence in that order. Rather, the terms “first uORF,” “second uORF,” “third uORF” and “fourth uORF” refer to uORFs in the 5′UTR of PKD1 mRNA that have the above sequences, or at the same or in similar locations in the 5′UTR of PKD1 mRNA and have homologous sequences (such as having about 80% or more, 85% or more, 90% or more, or 95% or more sequence identity).

Abbreviations: 5′UTR: 5′-untranslated region. ADPKD: autosomal dominant polycystic kidney disease. ADPLD: autosomal-dominant polycystic liver disease. ASO: antisense oligonucleotides. ER: endoplasmic reticulum. PCLD: polycystic liver disease. PKD: polycystic kidney disease. uORF: upstream open reading frame.

Method of Treating, Ameliorating and/or Preventing ADPKD or PCLD

In some aspects, the present invention is directed to a method of treating, ameliorating and/or preventing an autosomal dominant polycystic kidney disease (ADPKD) or a polycystic liver disease (PCLD) in a subject in need thereof.

In some embodiments, the method includes administering to the subject an effective amount of a compound that suppresses the translation or affects the secondary mRNA structure of the first upstream open reading frame (uORF), the second uORF, the third uORF or the fourth uORF of the PKD1 gene, or a combination of these.

In some embodiments, the method is a method of treating, ameliorating and/or preventing the ADPKD in the subject, and the ADPKD is caused by or involves a mutation in the PKD1 or PKD2 gene in the subject.

In some embodiments, the method is a method of treating, ameliorating and/or preventing the PCLD in the subject, and wherein the PCLD is caused by or involves a germline mutation of the PKD1 gene, the PKD2 gene, the PRKCSH gene, the SEC63 gene, the GANAB gene, the ALG8 gene, the ALG9 gene, the SEC61B gene, or the DNAJB11 gene, or other human disease genes including those not yet established for the ADPKD-PCLD spectrum of diseases in the subject.

In some embodiments, the compound that suppresses the first upstream open reading frame (uORF), the second uORF, the third uORF and/or the fourth uORF of the PKD1 gene includes:
CRISPR components that disrupt the genomic DNA sequence that encodes the first uORF, the second uORF, the third uORF and/or the fourth uORF, or an expression vector expressing the CRISPR components; or
an antisense oligonucleotide (ASO) that blocks the first uORF, the second uORF, the third uORF and/or the fourth uORF, or an expression vector expressing the ASO.

The present study has identified the sequence of the four uORFs in the 5′-UTR of the PKD1 mRNA, and confirmed that suppressing either or both of the first two uORFs by either altering the sequence of the uORFs or blocking the uORFs with ASOs is able to significantly increase the expression of PC1. It is expected that the third uORF and the fourth uORF in the PKD1 mRNA may function similarly or complentarily to the first and the second uORFs, and that abolishing or suppressing uORF3 and 4 would achieve similar or enhanced results.

One of ordinary skill in the art would understand that the technology of designing both CRISPR systems and ASOs are known in the art and that, with the target sequence of the uORFs identified by the present study and the description regarding CRISPR and ASOs herein (e.g., the “Suppressing PKD1 uORFs by CRISPR techniques” section and the “Suppressing PKD1 uORFs by ASO technology” section), CRISPR components and ASOs can be designed and obtained without undue experimentation.

In some embodiments, the compound includes the CRISPR components or the expression vector expressing the CRISPR components, and wherein the CRISPR components disrupt the initiation codon of the first uORF, the second uORF, the third uORF and/or the fourth uORF.

In some embodiments, the compound includes the ASO or the expression vector expressing the ASO, and the portion of the PKD1 mRNA complementary to the ASO extends to 5 nucleotides away or less from the initiation codon of the first uORF, the second uORF, the third uORF or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF, or the fourth uORF.

In some embodiments, the ASO suppresses the first uORF (“uORF 1”). In some embodiments, the ASO targets the entirety or a continuous portion of the sequence CCAGCCCCGCGCCCGCCAUGCCGUCCGCGGGCCCCGC (SEQ ID NO: 13, the initiation codon is highlighted), which includes the 5′-portion of uORF 1, as well as some nucleotides to the 5′-end thereof.

In some embodiments, the sequence of the 5′-UTR that is complementary (such as about 80% or more complementary, about 85% or more complementary, about 90% or more complementary, about 95% or more complementary, or fully complementary) to and targeted by the ASO is at least one from the following:

20-mer options
CCAGCCCCGCGCCCGCCAUG (SEQ ID NO: 14)
CAGCCCCGCGCCCGCCAUGC (SEQ ID NO: 15)
AGCCCCGCGCCCGCCAUGCC (SEQ ID NO: 16)
GCCCCGCGCCCGCCAUGCCG (SEQ ID NO: 17)
CCCCGCGCCCGCCAUGCCGU (SEQ ID NO: 18)
CCCGCGCCCGCCAUGCCGUC (SEQ ID NO: 19)
CCGCGCCCGCCAUGCCGUCC (SEQ ID NO: 20)
CGCGCCCGCCAUGCCGUCCG (SEQ ID NO: 21)
GCGCCCGCCAUGCCGUCCGC (SEQ ID NO: 22)
CGCCCGCCAUGCCGUCCGCG (SEQ ID NO: 23)
GCCCGCCAUGCCGUCCGCGG (SEQ ID NO: 24)
CCCGCCAUGCCGUCCGCGGG (SEQ ID NO: 25)
CCGCCAUGCCGUCCGCGGGC (SEQ ID NO: 26)
CGCCAUGCCGUCCGCGGGCC (SEQ ID NO: 27)
GCCAUGCCGUCCGCGGGCCC (SEQ ID NO: 28)
CCAUGCCGUCCGCGGGCCCC (SEQ ID NO: 29)
CAUGCCGUCCGCGGGCCCCG (SEQ ID NO: 30)
AUGCCGUCCGCGGGCCCCGC (SEQ ID NO: 31)
18-mer options
AGCCCCGCGCCCGCCAUG (SEQ ID NO: 32)
GCCCCGCGCCCGCCAUGC (SEQ ID NO: 33)
CCCCGCGCCCGCCAUGCC (SEQ ID NO: 34)
CCCGCGCCCGCCAUGCCG (SEQ ID NO: 35)
CCGCGCCCGCCAUGCCGU (SEQ ID NO: 36)
CGCGCCCGCCAUGCCGUC (SEQ ID NO: 37)
GCGCCCGCCAUGCCGUCC (SEQ ID NO: 38)
CGCCCGCCAUGCCGUCCG (SEQ ID NO: 39)
GCCCGCCAUGCCGUCCGC (SEQ ID NO: 40)
CCCGCCAUGCCGUCCGCG (SEQ ID NO: 41)
CCGCCAUGCCGUCCGCGG (SEQ ID NO: 42)
CGCCAUGCCGUCCGCGGG (SEQ ID NO: 43)
GCCAUGCCGUCCGCGGGC (SEQ ID NO: 44)
CCAUGCCGUCCGCGGGCC (SEQ ID NO: 45)
CAUGCCGUCCGCGGGCCC (SEQ ID NO: 46)
AUGCCGUCCGCGGGCCCC (SEQ ID NO: 47)
16-mer options
CCCCGCGCCCGCCAUG (SEQ ID NO: 48)
CCCGCGCCCGCCAUGC (SEQ ID NO: 49)
CCGCGCCCGCCAUGCC (SEQ ID NO: 50)
CGCGCCCGCCAUGCCG (SEQ ID NO: 51)
GCGCCCGCCAUGCCGU (SEQ ID NO: 52)
CGCCCGCCAUGCCGUC (SEQ ID NO: 53)
GCCCGCCAUGCCGUCC (SEQ ID NO: 54)
CCCGCCAUGCCGUCCG (SEQ ID NO: 55)
CCGCCAUGCCGUCCGC (SEQ ID NO: 56)
CGCCAUGCCGUCCGCG (SEQ ID NO: 57)
GCCAUGCCGUCCGCGG (SEQ ID NO: 58)
CCAUGCCGUCCGCGGG (SEQ ID NO: 59)
CAUGCCGUCCGCGGGC (SEQ ID NO: 60)
AUGCCGUCCGCGGGCC (SEQ ID NO: 61)

In some embodiments, the ASO suppresses uORF 2. In some embodiments, the ASO targets the entirety or a portion of the sequence CUGGGGACGGCGGGGCCAUGCGCGCGCUGCCCUAACG (SEQ ID NO:, the initiation codon is highlighted), which includes the 5′-portion of uORF 2, as well as some nucleotides to the 5′-end thereof.

In some embodiments, the sequence of the 5′-UTR that is complementary (such as about 80% or more complementary, about 85% or more complementary, about 90% or more complementary, about 95% or more complementary, or fully complementary) to and targeted by the ASO is at least one from the following:

20-mer options
CUGGGGACGGCGGGGCCAUG (SEQ ID NO: 62)
UGGGGACGGCGGGGCCAUGC (SEQ ID NO: 63)
GGGGACGGCGGGGCCAUGCG (SEQ ID NO: 64)
GGGACGGCGGGGCCAUGCGC (SEQ ID NO: 65)
GGACGGCGGGGCCAUGCGCG (SEQ ID NO: 66)
GACGGCGGGGCCAUGCGCGC (SEQ ID NO: 67)
ACGGCGGGGCCAUGCGCGCG (SEQ ID NO: 68)
CGGCGGGGCCAUGCGCGCGC (SEQ ID NO: 69)
GGCGGGGCCAUGCGCGCGCU (SEQ ID NO: 70)
GCGGGGCCAUGCGCGCGCUG (SEQ ID NO: 71)
CGGGGCCAUGCGCGCGCUGC (SEQ ID NO: 72)
GGGGCCAUGCGCGCGCUGCC (SEQ ID NO: 73)
GGGCCAUGCGCGCGCUGCCC (SEQ ID NO: 74)
GGCCAUGCGCGCGCUGCCCU (SEQ ID NO: 75)
GCCAUGCGCGCGCUGCCCUA (SEQ ID NO: 76)
CCAUGCGCGCGCUGCCCUAA (SEQ ID NO: 77)
CAUGCGCGCGCUGCCCUAAC (SEQ ID NO: 78)
AUGCGCGCGCUGCCCUAACG (SEQ ID NO: 79)
18-mer options
GGGGACGGCGGGGCCAUG (SEQ ID NO: 80)
GGGACGGCGGGGCCAUGC (SEQ ID NO: 81)
GGACGGCGGGGCCAUGCG (SEQ ID NO: 82)
GACGGCGGGGCCAUGCGC (SEQ ID NO: 83)
ACGGCGGGGCCAUGCGCG (SEQ ID NO: 84)
CGGCGGGGCCAUGCGCGC (SEQ ID NO: 85)
GGCGGGGCCAUGCGCGCG (SEQ ID NO: 86)
GCGGGGCCAUGCGCGCGC (SEQ ID NO: 87)
CGGGGCCAUGCGCGCGCU (SEQ ID NO: 88)
GGGGCCAUGCGCGCGCUG (SEQ ID NO: 89)
GGGCCAUGCGCGCGCUGC (SEQ ID NO: 90)
GGCCAUGCGCGCGCUGCC (SEQ ID NO: 91)
GCCAUGCGCGCGCUGCCC (SEQ ID NO: 92)
CCAUGCGCGCGCUGCCCU (SEQ ID NO: 93)
CAUGCGCGCGCUGCCCUA (SEQ ID NO: 94)
AUGCGCGCGCUGCCCUAA (SEQ ID NO: 95)
16-mer options
GGACGGCGGGGCCAUG (SEQ ID NO: 96)
GACGGCGGGGCCAUGC (SEQ ID NO: 97)
ACGGCGGGGCCAUGCG (SEQ ID NO: 98)
CGGCGGGGCCAUGCGC (SEQ ID NO: 99)
GGCGGGGCCAUGCGCG (SEQ ID NO: 100)
GCGGGGCCAUGCGCGC (SEQ ID NO: 101)
CGGGGCCAUGCGCGCG (SEQ ID NO: 102)
GGGGCCAUGCGCGCGC (SEQ ID NO: 103)
GGGCCAUGCGCGCGCU (SEQ ID NO: 104)
GGCCAUGCGCGCGCUG (SEQ ID NO: 105)
GCCAUGCGCGCGCUGC (SEQ ID NO: 106)
CCAUGCGCGCGCUGCC (SEQ ID NO: 107)
CAUGCGCGCGCUGCCC (SEQ ID NO: 108)
AUGCGCGCGCUGCCCU (SEQ ID NO: 109)

In some embodiments, the ASO suppresses uORF 3. In some embodiments, the ASO targets the entirety or a portion of the sequence CGAGCTCCCGGAGCGGCCTGGCCCCGAGCCCCGAGCG (SEQ ID NO:, the initiation codon is highlighted), which includes the 5′-portion of uORF 3, as well as some nucleotides to the 5′-end thereof.

In some embodiments, the sequence of the 5′-UTR that is complementary (such as about 80% or more complementary, about 85% or more complementary, about 90% or more complementary, about 95% or more complementary, or fully complementary) to and targeted by the ASO is at least one from the following:

20-mer options
CGAGCUCCCGGAGCGGCCUG (SEQ ID NO: 110)
GAGCUCCCGGAGCGGCCUGG (SEQ ID NO: 111)
AGCUCCCGGAGCGGCCUGGC (SEQ ID NO: 112)
GCUCCCGGAGCGGCCUGGCC (SEQ ID NO: 113)
CUCCCGGAGCGGCCUGGCCC (SEQ ID NO: 114)
UCCCGGAGCGGCCUGGCCCC (SEQ ID NO: 115)
CCCGGAGCGGCCUGGCCCCG (SEQ ID NO: 116)
CCGGAGCGGCCUGGCCCCGA (SEQ ID NO: 117)
CGGAGCGGCCUGGCCCCGAG (SEQ ID NO: 118)
GGAGCGGCCUGGCCCCGAGC (SEQ ID NO: 119)
GAGCGGCCUGGCCCCGAGCC (SEQ ID NO: 120)
AGCGGCCUGGCCCCGAGCCC (SEQ ID NO: 121)
GCGGCCUGGCCCCGAGCCCC (SEQ ID NO: 122)
CGGCCUGGCCCCGAGCCCCG (SEQ ID NO: 123)
GGCCUGGCCCCGAGCCCCGA (SEQ ID NO: 124)
GCCUGGCCCCGAGCCCCGAG (SEQ ID NO: 125)
CCUGGCCCCGAGCCCCGAGC (SEQ ID NO: 126)
CUGGCCCCGAGCCCCGAGCG (SEQ ID NO: 127)
18-mer options
AGCUCCCGGAGCGGCCUG (SEQ ID NO: 128)
GCUCCCGGAGCGGCCUGG (SEQ ID NO: 129)
CUCCCGGAGCGGCCUGGC (SEQ ID NO: 130)
UCCCGGAGCGGCCUGGCC (SEQ ID NO: 131)
CCCGGAGCGGCCUGGCCC (SEQ ID NO: 132)
CCGGAGCGGCCUGGCCCC (SEQ ID NO: 133)
CGGAGCGGCCUGGCCCCG (SEQ ID NO: 134)
GGAGCGGCCUGGCCCCGA (SEQ ID NO: 135)
GAGCGGCCUGGCCCCGAG (SEQ ID NO: 136)
AGCGGCCUGGCCCCGAGC (SEQ ID NO: 137)
GCGGCCUGGCCCCGAGCC (SEQ ID NO: 138)
CGGCCUGGCCCCGAGCCC (SEQ ID NO: 139)
GGCCUGGCCCCGAGCCCC (SEQ ID NO: 140)
GCCUGGCCCCGAGCCCCG (SEQ ID NO: 141)
CCUGGCCCCGAGCCCCGA (SEQ ID NO: 142)
CUGGCCCCGAGCCCCGAG (SEQ ID NO: 143)
16-mer options
CUCCCGGAGCGGCCUG (SEQ ID NO: 144)
UCCCGGAGCGGCCUGG (SEQ ID NO: 145)
CCCGGAGCGGCCUGGC (SEQ ID NO: 146)
CCGGAGCGGCCUGGCC (SEQ ID NO: 147)
CGGAGCGGCCUGGCCC (SEQ ID NO: 148)
GGAGCGGCCUGGCCCC (SEQ ID NO: 149)
GAGCGGCCUGGCCCCG (SEQ ID NO: 150)
AGCGGCCUGGCCCCGA (SEQ ID NO: 151)
GCGGCCUGGCCCCGAG (SEQ ID NO: 152)
CGGCCUGGCCCCGAGC (SEQ ID NO: 153)
GGCCUGGCCCCGAGCC (SEQ ID NO: 154)
GCCUGGCCCCGAGCCC (SEQ ID NO: 155)
CCUGGCCCCGAGCCCC (SEQ ID NO: 156)
CUGGCCCCGAGCCCCG (SEQ ID NO: 157)

In some embodiments, in an ASO that is “fully complementary to” one of the sequences set forth in SEQ ID NOs: 14-157, every nucleobase is involved in forming hybridization with a nucleobase of that sequence, and vice versa.

In some embodiments, a length of the ASO is 10 nucleotides or longer, such as 11 nucleotides or longer, 12 nucleotides or longer, 13 nucleotides or longer, 14 nucleotides or longer or 15 nucleotides or longer.

In some embodiments, a length of the ASO is 30 nucleotides or shorter, such as 29 nucleotides or shorter, 28 nucleotides or shorter, 27 nucleotides or shorter, 26 nucleotides or shorter or 25 nucleotides or shorter.

In some embodiments, the ASO is an RNA molecule, a DNA molecule, or a DNA-RNA hybrid molecule. In some embodiments, the ASO includes modified nucleobase(s), modified linkage(s) and/or modified sugar group(s).

In some embodiments, the modified sugar groups include a 2′-O-methylation (2′-OMe) modified sugar group, a locked nucleic acid (LNA) modified sugar group, a 2′-O-methoxyethyl (2′-MOE) modified sugar group, a (S)-constrained ethyl nucleic acid (cEt) modified sugar group, or a 2′fluoro (2′F) modified sugar group.

In some embodiments, the modified linkage includes a phosphorothioate (PS) linkage, a phosphorodiamidate morpholino (PMO) linkage, a positively charged PMO linkage, a phosphoramidate linkage, a methylphosphonate (MP) linkage, a phosphorothioate linkage, or a peptide nucleic acid (PNA) linkage. In some embodiments, the ASO includes a combination of the modified linkages and the natural phosphodiester (PO) linkages, such as a combination of the PS linkages and the PO linkages. In some embodiments the alterations may lead to its characterization as a PMO morpholino.

In some embodiments, the modified nucleobase includes a 5′methylcytosine nucleobase, or a G-clamp nucleobase.

In some embodiments, the ASO is conjugated to an N-acetylgalactosamine (GalNAc) group, or a cholesterol group. In some embodiments, the conjugation improves the delivery of the ASO, such as the delivery across cell membrane.

In some embodiments, the ASO is of a Gapmer design. In some embodiments, the Gapmer includes a DNA-based internal “gap” and RNA-like flanking regions. In some embodiments, the RNA-like flanking regions includes one or more modifications, such as those described above, such as 2′-OMe or LNA modifications.

In some embodiments, the ASO includes the nucleotide sequence CAUGGCGGGCGCGGGG (SEQ ID NO:158), the nucleotide sequence CAUGGCCCCGCCGUCC (SEQ ID NO: 159), or variations thereof. It is worth noting that, since the ASO can be RNA-based, DNA-based or DNA/RNA hybrid-based, and can include modifications in the nucleobases, linkages or sugar groups, the ASO molecules herein are not intended to be limited by the nature of the molecules as suggested by the listed sequence. For example, while the sequence CAUGGCGGGCGCGGGG (SEQ ID NO:158) suggests that this ASO molecule is an RNA molecule, this sequence is intended to include the corresponding DNA molecules, the corresponding DNA/RNA hybrid molecules, as well as the corresponding nucleic acids including modified nucleobases, linkages and/or sugar groups, as well.

In some embodiments, a concentration of the ASO in the affected tissues (kidney or lung) after the administration ranges from about 1 nm to about 100 nm, such as about 2 nm to about 75 nm, about 3 nm to about 50 nm, about 4 nm to about 40 nm, about 5 nm to about 30 nm, or about 10 to about 20 nm.

In some embodiments, the subject is a mammal, such as a human.

### Method of Increasing PKD1/Polycystin-1 (PC1) Expression in Cell

In some embodiments, the instant specification is directed to a method of increasing PKD1/PC1 expression in a cell.

In some embodiments, the method includes: contacting with the cell an effective amount of a compound that suppresses the first upstream open reading frame (uORF) and/or the second uORF of the PKD1 gene.

In some embodiments, the cell has a mutation in the PKD1 gene, the PKD2 gene, the PRKCSH gene, the SEC63 gene, the GANAB gene, the ALG8 gene, the ALG9 gene, the SEC61B gene, or the DNAJB11 gene.

In some embodiments, the cell is in a tissue or a subject.

In some embodiments, the cell is a kidney cell in a subject diagnosed with autosomal dominant polycystic kidney disease (ADPKD) or a liver cell in a subject diagnosed with polycystic liver disease (PCLD).

In some embodiments, the compound includes:
CRISPR components that disrupt the genomic DNA sequence that encoding the first uORF and/or the second uORF, or an expression vector expressing the CRISPR components; or
an antisense oligonucleotide (ASO) that blocks the first uORF and/or the second uORF, or an expression vector expressing the ASO.

Such CRISPR components and ASOs are detailed elsewhere herein.

In some embodiments, the compound includes the CRISPR components or the expression vector expressing the CRISPR components, and the CRISPR components disrupt the initiation codon of the first uORF and/or the second uORF.

In some embodiments, the compound includes the ASO or the expression vector expressing the ASO, and the portion of the PKD1 mRNA complementary to the ASO extends to 5 nucleotides away or less from the initiation codon of the first uORF or the second uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF or the second uORF.

In some embodiments, a length of the ASO is 10 nucleotides or longer, such as 11 nucleotides or longer, 12 nucleotides or longer, 13 nucleotides or longer, 14 nucleotides or longer or 15 nucleotides or longer.

In some embodiments, a length of the ASO is 30 nucleotides or shorter, such as 29 nucleotides or shorter, 28 nucleotides or shorter, 27 nucleotides or shorter, 26 nucleotides or shorter or 25 nucleotides or shorter.

In some embodiments, the ASO includes the nucleotide sequence CAUGGCGGGCGCGGGG (SEQ ID NO:158), the nucleotide sequence CAUGGCCCCGCCGUCC (SEQ ID NO: 159), or variations thereof including modified nucleobases or modified linkages.

In some embodiments, the modified nucleobases include 2′-O-methylation modified nucleobases.

In some embodiments, the modified linkages include phosphorothioate (PS) linkages.

In some embodiments, the concentration of the ASO contacted with the cell ranges from about 1 nm to about 100 nm, such as about 2 nm to about 75 nm, about 3 nm to about 50 nm, about 4 nm to about 40 nm, about 5 nm to about 30 nm, or about 10 to about 20 nm.

Suppressing PKD1 uORFs by CRISPR Techniques

In some embodiments, the CRISPR components are designed to induce targeted genetic alterations in the sequence of the uORFs in the 5′-UTR of PKD1 gene, such as in the initiation codon (“AUG/ATG”) of the uORFs in the genomic DNA.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the present invention includes any other methods for effecting gene knockdown and/editing, which allow for deletion and/or inactivation of the uORFs, such as but not limited to those described in WO 2018/236840 (which is incorporated herein in its entirety by reference).

Suppressing PKD1 uORFs by ASO Technology

In some embodiments, the ASO molecules are designed such that the ASOs hybridize with the 5′-UTR of PKD1 mRNA at or near the uORFs (such as at or near the initiation codons of the uORFs) such that the ribosome binding efficiencies of the uORFs are substantially reduced.

An antisense oligonucleotide (ASO) is a short strand of nucleotide analogue that hybridizes with the complementary mRNA in a sequence-specific manner. The technology was first described in 1970s and has been developed since 1980s by incorporating the advances in oligonucleotide chemistry and formulations. Currently, there are several approved ASO drugs and a significant number of ASO drugs in development. (Bennett, Annual Review of Medicine, Vol. 70:307-321, 2019).

ASOs are normally 13-25 nucleotides long, but are not limited thereto. ASOs are complementary to the mRNA they are targeting and are able to hybridize to the mRNA through Watson-Crick base pairing. The sequence of the ASO can be designed with the aid of available software and algorithms. The design of ASO targeting certain sequence is described in, for example, Chan et al. (CEPP, Volume 33, Issue 5-6 May/June 2006, Pages 533-540).

The formation of mRNA/ASO duplex can cause two effects: 1) ribonuclease H (RNaseH)-mediated cleavage, which degrades the duplex, and 2) steric hindrance, which limits the access of ribosome to the segment of the mRNA complementary with the ASO.

Because, according to certain embodiments, it is desirable to provide steric hindrance to limit the access of the uORFs in the 5′-UTR of PKD1 mRNA (which would increase the translation of PC1 protein from PKD1 mRNA) without causing the degradation of the mRNA (which would reduce the expression of PKD1 mRNA and its translation to PC1 protein), in some embodiments, the ASO herein include chemical modifications that would render the formed RNA/ASO duplexes resistant to endonuclease cleavage. Sugar modifications, such as 2′-O-methyl (2′-OMe) or 2′-O-methoxyethyl (2′-O-MOE) modifications and phosphorothioate modifications, and linkage modifications such as phosphorothioate (PS) modifications are known to increase the resistance of the RNA/ASO duplex to endonuclease cleavage (see e.g., Gagliardi et al., Biomedicines. 2021 April; 9 (4): 433 and Crooke et al., Nucleic Acids Res. 2020 Jun. 4; 48 (10): 5235-5253). Therefore, in some embodiments, the ASOs herein includes one of more of such modifications.

The design of ASOs to improve delivery is also described in Roberts et al. (Nature Reviews Drug Discovery volume 19, pages 673-694 (2020)).

### Vectors

Vectors can increase the stability of the nucleic acids, make the delivery easier, or allow the expression of the nucleic acids or protein products thereof in the cells.

Therefore, in some embodiments, the compound that suppresses PKD1 uORFs is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the compounds described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the liver. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the polycystic diseases includes administering to the subject the effective amount of at least one compound and/or composition contemplated within the disclosure.

In some embodiments, the composition for treating the polycystic diseases includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating the polycystic diseases, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of the polycystic diseases in a patient.

Formulations may be employed in admixtures with conventional excipients, i.e., pharmaceutically acceptable organic or inorganic carrier substances suitable for intracranially, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, trans mucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the polycystic diseases in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Overview

PCLD, as well as a significant subset of ADPKD are caused by insufficient PC1 functional dosage, which are caused by either loss-of-function mutations of the PKD1 gene per se, or mutations of endoplasmic reticulum (ER) genes responsible for the maturation of PC1 from the ER to the cell surface of the primary cilium. Therefore, increasing the functional dosage of PC1 is expected to be able to treat, ameliorate or prevent PCLD and ADPKD.

As described in Example 1, the study described herein (“the present study”) discovered two upstream open reading frames (uORFs) in the 5′-untranslated region (5′-UTR) of the PKD1 messenger RNA (mRNA). The present study further discovered that the two uORFs reduce translation efficiency of PKD1, and that either abolishing the uORFs through mutations or blocking the uORFs using antisense oligonucleotides (ASOs) significantly enhanced the translation of the PKD1 mRNA and increases the protein level of PC1.

Specifically, the in vitro data described herein demonstrated an approximately 4-fold increase in steady-state expression of the downstream gene when the PKD1 5′UTR has just 2 nucleotide edits to abolish uORF translation. The present study also shows that inhibition of PKD1 uORF translation in human cells using antisense oligonucleotides (ASOs) nearly recapitulate the fold-change of expression in the base-edited evaluation, thus this highly specific therapy is also highly achievable.

Since the two uORFs in the 5′-UTR of PKD1 have unique sequences, and methods targeting the uORFs sequences, such as CRISPR or ASOs, can be designed with extremely high specificity, treatments of ADPKD or PCLD through the uORFs are expected to be highly specific. Considering the fold-charge in PKD1 protein expressions achieved by inhibiting the uORFs, it is hypothesized that this magnitude of an effect would be sufficient to prevent cyst formation in at least some models or patients, and would be a safe approach to long-term therapy. Also, it is expected the method would be effective for all patients with isolated polycystic liver disease (symptomatic incidence ˜1:10,000-1:150,000) and a subset of patients with ADPKD (total incidence ˜1:400-1:1000).

### Example 1-1: Related Information

Autosomal dominant polycystic kidney disease (ADPKD) affects 1:400-1:1000, causing 5-10% of kidney failure. ADPKD results from pathogenic mutations in the PKD1 or PKD2 genes which encode polycystin 1 (PC1) and polycystin 2 (PC2), and is characterized by enlarging kidney and liver cysts. An overlapping spectrum of kidney and liver cystic phenotypes, ADPKD-ADPLD (also referred to as isolated polycystic liver disease or PCLD), are caused by mutations in genes necessary for PC1 maturation in the endoplasmic reticulum (ER): SEC63, PRKCSH, ALG8, ALG9, GANAB, SEC61B, DNAJB117. PKD1 truncating mutations which result in complete loss of PC1 function result in the most rapid progression to kidney failure. Patients with non-truncating PKD1 mutations nonetheless progress to kidney failure and these and many ADPKD-ADPLD cases are in desperate need of a therapy to stop the symptoms and complications of relentless accumulation and expansion of kidney and/or liver cysts. The later onset of kidney failure in those with non-truncating PKD1 mutations—approximately one third of ADPKD-suggest at least a partial function of the encoded PC1. A significant subset of these missense mutant PC1 constructs inefficiently mature to the cell surface, suggesting that the quantitative expression is critical. Interestingly, in cystic kidney mouse models of impaired ER maturation—Prkcsh or Sec63—that a quantitative increase in PC1 production, achieved by the mouse carrying a bacterial artificial chromosome encoding three copies of the Pkd1 gene, was sufficient to get enough PC1 to the cell surface to avoid cyst formation. Together, these findings suggest that in the face of inefficient PC1 maturation, increasing the PC1 expression can successfully increase the functional amount that gets to the cell surface to an extent that could prevent cyst formation.

An investigation in the University of Texas Southwestern has suggested an approach to inhibit microRNAs that degrade PKD1 mRNA. The genetic mouse models have indeed shown benefit from the resulting increase in PC1 expression from this approach (Lakhia et al. Nat Comm. 2022).

However, the microRNA approach would target a huge number of genes and therapies based on this will be profoundly limited by off-target effects. For this reason, it is believed that the specificity of targeting the unique sequences of PKD1 uORFs, in conjunction with a predicted similar-fold, if not more, increase in PC1 protein expression will have similar, if not more, desirable efficacy without the risks.

### Example 1-2: PKD1 mRNA Includes at Least Three Upstream Open Reading Frames (uORFs) in the 5′UTR

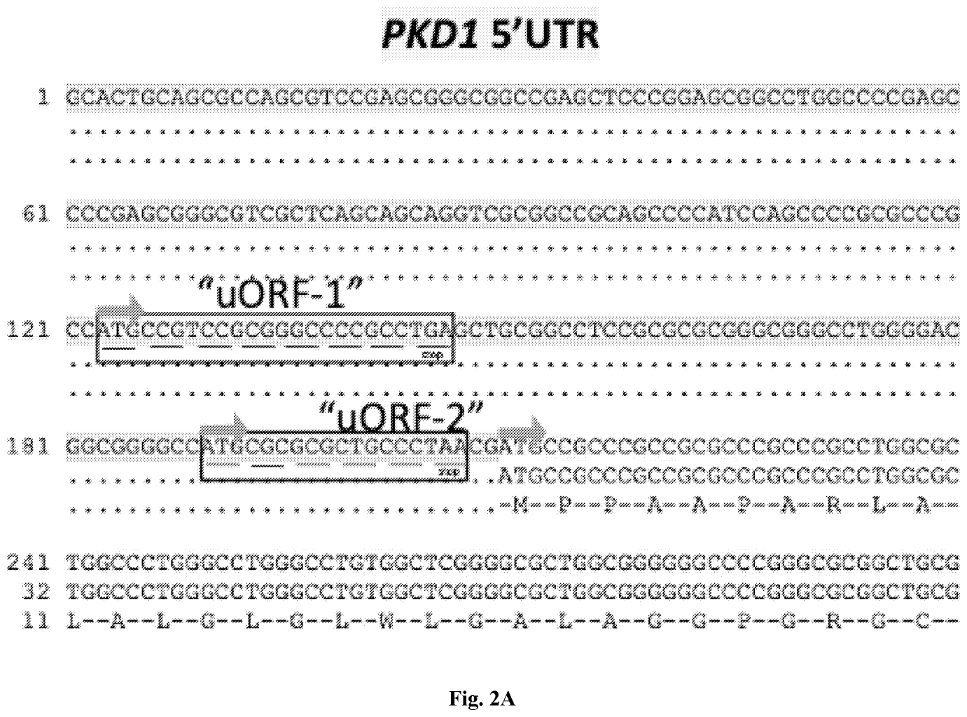

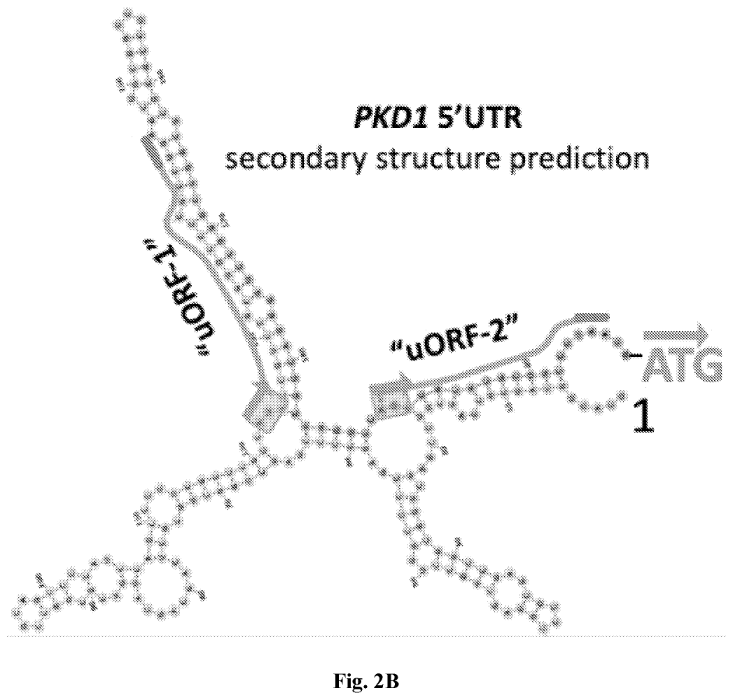

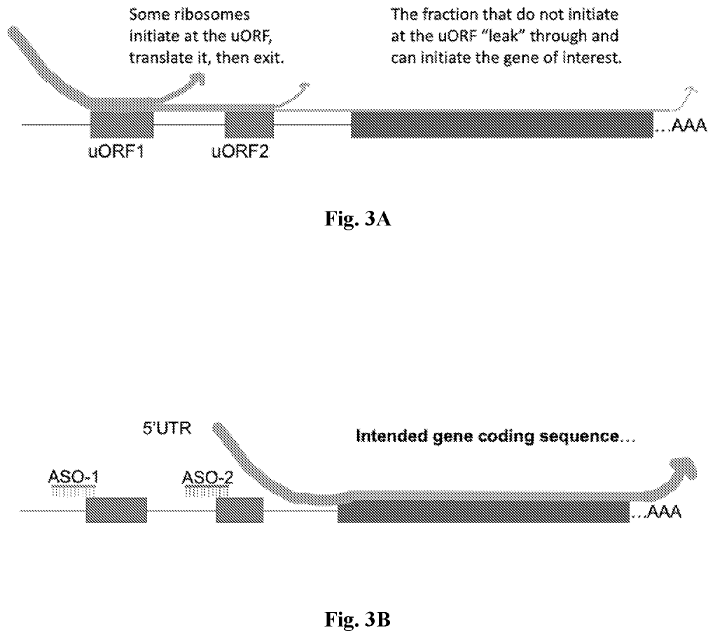

In a computationally-determined scores of the likelihood of uORFs translation, three potential uORFs in the PKD1 5′-untranslated region (5′UTR) were identified among the top 0.1% of all scored genome-wide uORFs. The sequences of these uORFs (uORF1, uORF2, uORF3) as well as a proposed uORF4 are set forth in SEQ ID NO: 3 and SEQ ID NO:4, SEQ ID NO:5, SEQ ID NO:6 respectively. The two ATG-initiated uORFs (uORF1 and uORF2) are shown in FIG. 2A. The uORFs, as well as the predicted secondary structure of the 5′UTR, is further illustrated in FIG. 2B.

The 5′ untranslated region (5′UTR) of messenger RNA (mRNA) sequence from many genes contain potential translation start sites encoding short or out of frame peptides from the intended gene; these are known as upstream open reading frames (uORFs). For some of the uORFs studied for other genes, translation of uORFs has a deleterious effect on translation of the intended protein by reducing the number of ribosomes that reach the initiation codon of the protein sequence (FIGS. 3A-3B).

### Example 1-3: Genetically Abolishing uORFs in PKD1 5′UTR Significantly Increases Protein Expression

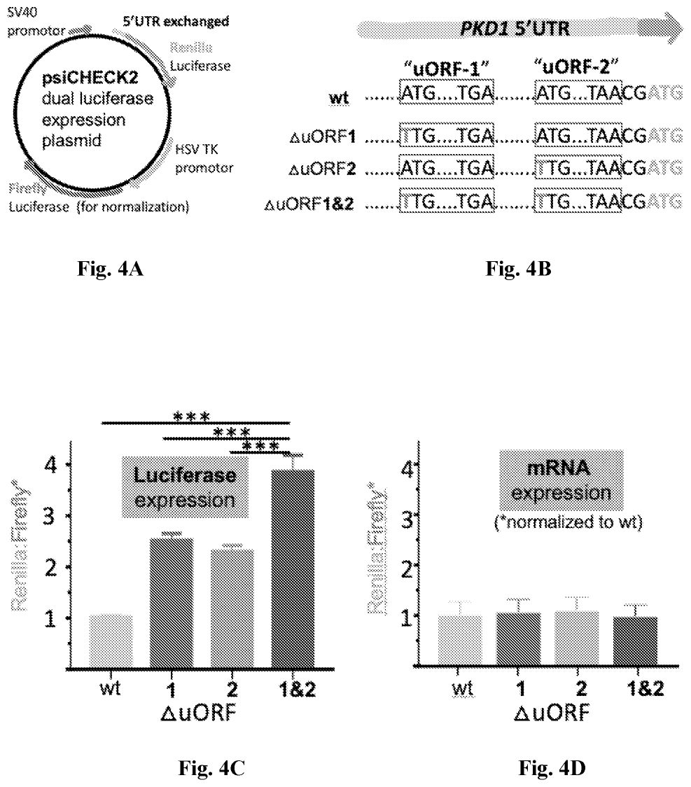

Referring to FIGS. 4A-4D, constructs were prepared by replacing the 5′UTR of Renilla luciferase in the dual luciferase reporter (FIG. 4A) with either wild-type human PKD1 5′UTR (“wt”) or human PKD1 5′UTRs containing either one or two single base edits to abolish either one or both of the uORF start sites (“ΔuORF1,” “ΔuORF2,” and “ΔuORF1&2”) (FIG. 4B). Cells were then transfected with the four constructs. The mutations that abolishing the uORFs did not alter the mRNA expression or transfection efficiency (FIG. 4D). However, this targeted edit resulted in a significant-nearly 4-fold-increased expression of the luciferase protein (FIG. 4C, “wt” vs “1&2”).

### Example 1-4: ASOs Inhibiting uORFs in PKD1 5′UTR Significantly Increases Protein Expression

Based on the sequences of the uORFs as well as the sequence of the 5′UTR, two non-limiting examples of antisense oligonucleotides were designed: uORF1 ASO (“ASO1”) (CAUGGCGGGCGCGGGG, SEQ ID NO:158, complementary with the 5′UTR sequence preceding and including the AUG initiation codon of uORF1), and uORF2 ASO (“ASO2”) (CAUGGCCCCGCCGUCC, SEQ ID NO:159, complementary with the 5′UTR sequence preceding and including the AUG initiation codon of uORF2). Both ASOs include 2′-O-methylation modified nucleobases, as well as phosphorothioate (PS) linkages to increase resistance to degradation.

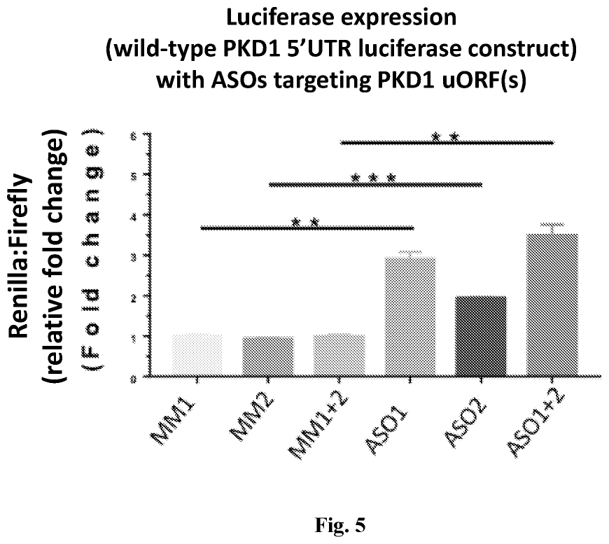

After transfection of cells expressing the luciferase reporter including the wild-type 5′UTR of PKD1 (see “Example 1-3” section), cells were incubated with the either one or both of the ASOs (ASO1, ASO2, or ASO1+2), or with mismatched controls (MM1, MM2) ASOs. Lysates were collected after 24 hours of treatment. Referring to FIG. 5, the luciferase expression in lysate of cells treated with both uORF1 and uORF2 ASOs showed approximately 3-fold increase when comparing to the lysates of cells treated with controls.

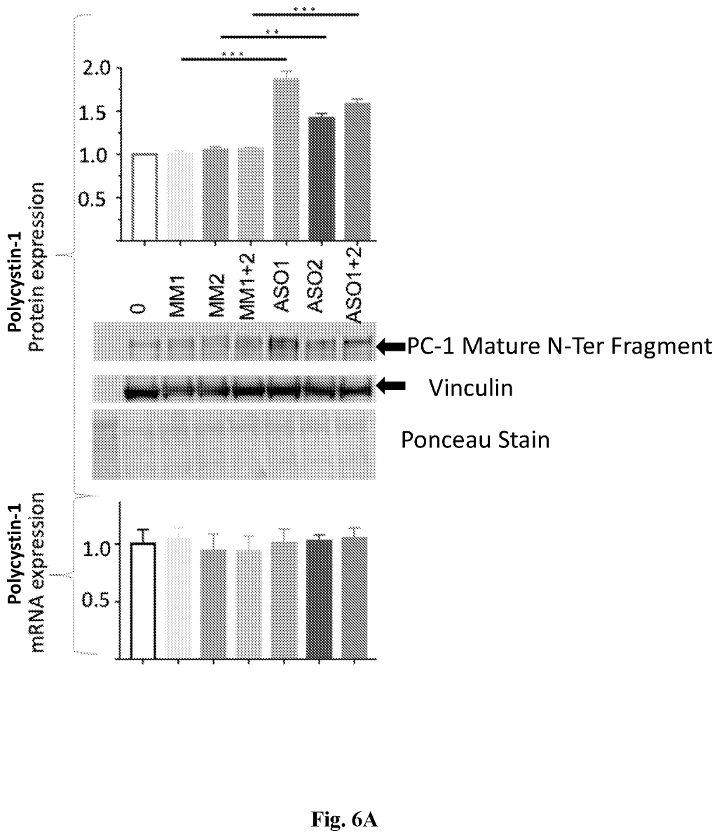

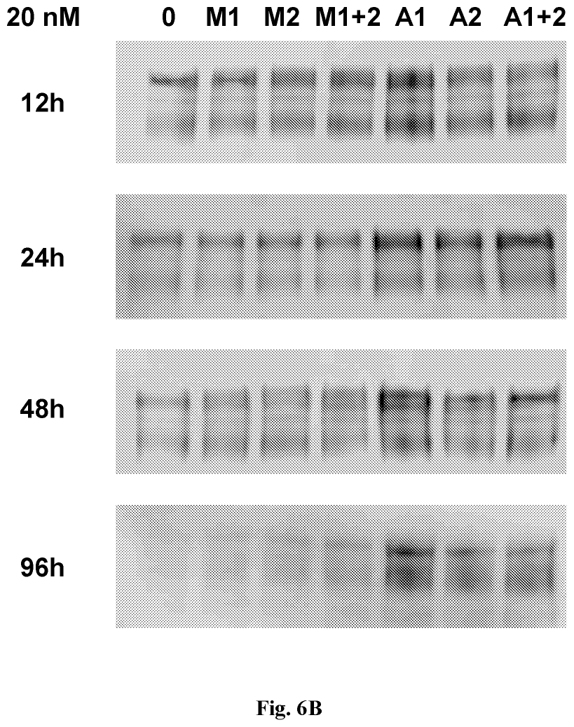

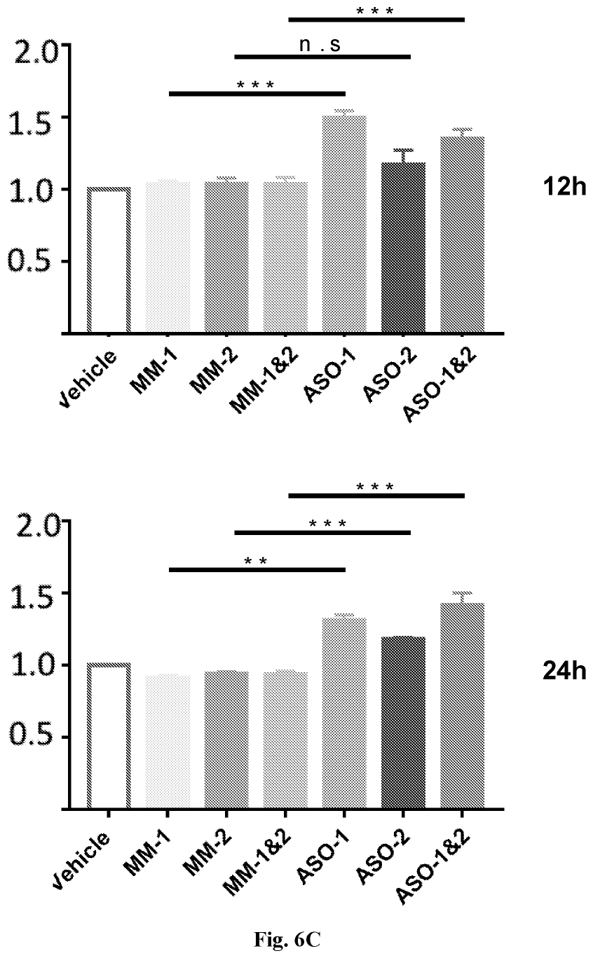

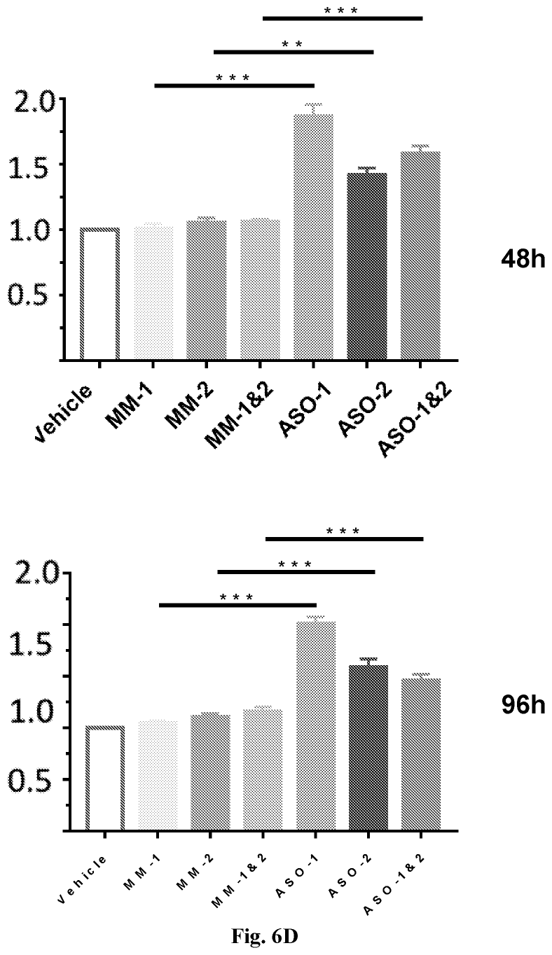

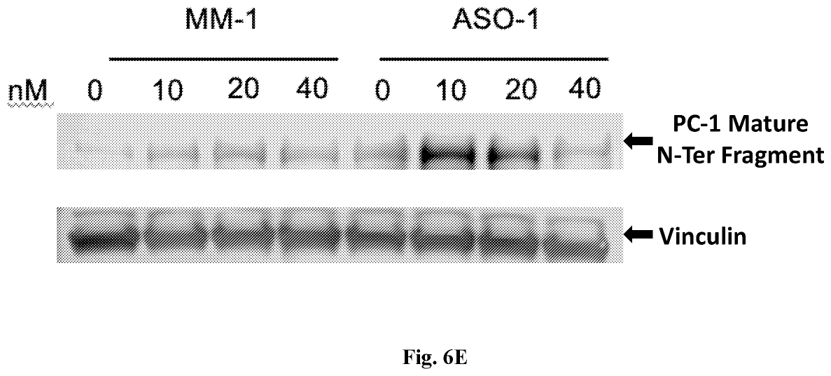

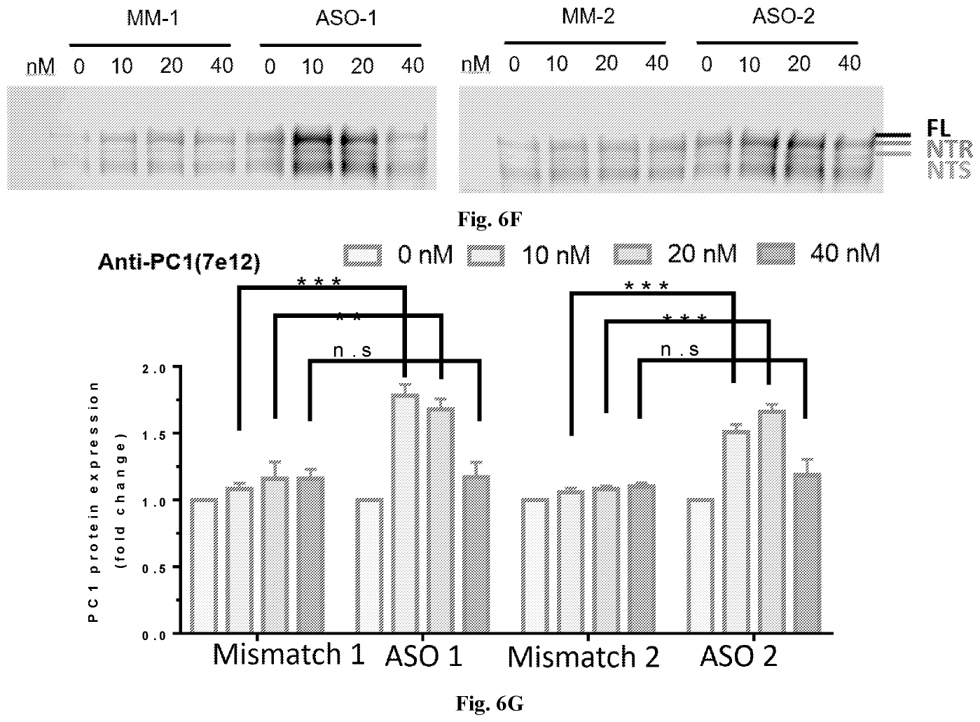

Referring to FIG. 6A, wild-type epithelial cells were treated with 20 nm of ASOs, and 1.5-2-fold increase in PC1 steady-state expression level compared with that seen in mismatched ASOs were observed without any significant change in mRNA expression. This is consistent with the proposed mechanism of uORF inhibition promoting translation without effect on transcript. The increase in PC1 expression can be observed with 12-, 24-, 48- and 96-hours of treatment (FIGS. 6B-6D). Referring to FIG. 6E, when the concentrations of ASOs were 10 nm or 20 nm, strong upregulation of PC1 protein expression was observed.

### Example 1-5: Design of ASOs for Sterically Blocking Mouse Pkd1 uORF Translation

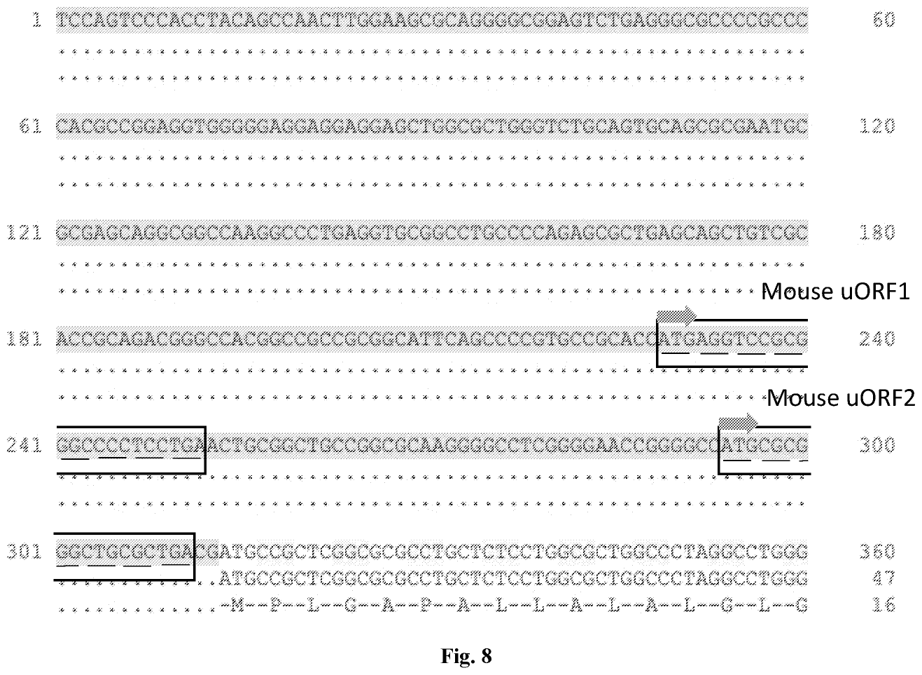

Referring to FIG. 8, the examination of the mouse Pkd1 mRNA sequence reveals two ATG-initiated potential uORFs (mouse uORF1 and mouse uORF2) corresponding to the two human PKD1 uORFs.

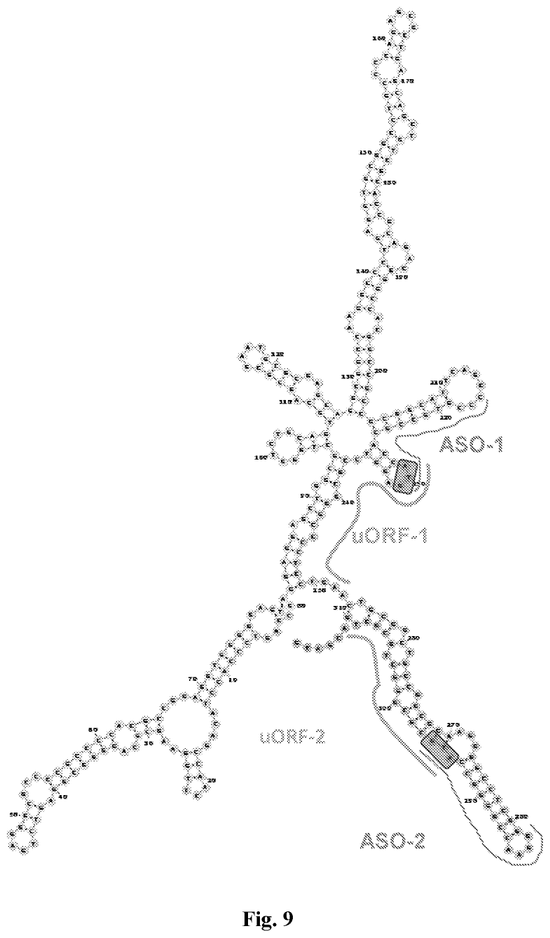

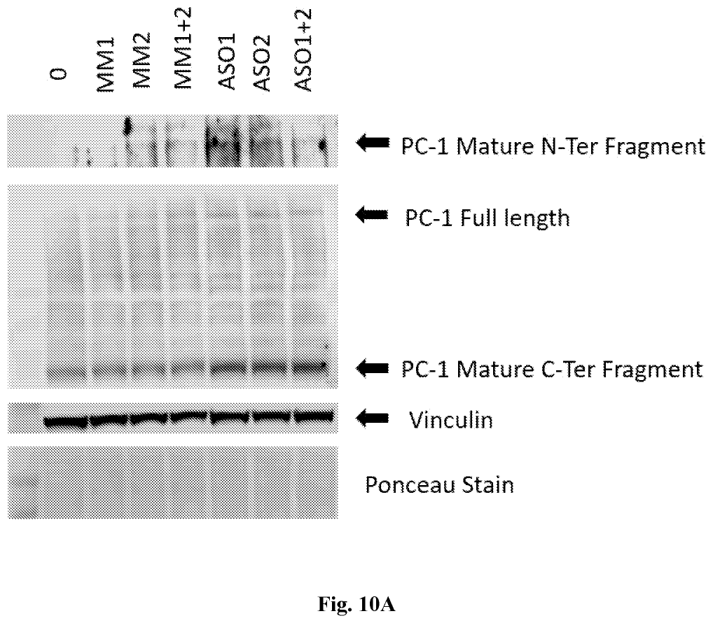

Referring to FIG. 9, two ASOs for suppressing the mouse uORFs (mouse ASO1 and mouse ASO2), as well as two mismatched control ASOs (mouse MM-1 and mouse MM-2) were designed using published information as guidance (Liang et al. Nat Biotechnol 34, 875-880 (2016) and Liang et al. (Nucleic acids research 45, 9528-9546 (2017)). Specifically, the ASOs, each having a length of 16 nucleotides and including 2′-O-methyl (2′-OMe) modifications and phosphorothioate (PS) linkages, were designed to have the following sequences: Mouse uORF-1 ASO (mouse ASO1): 5′ mC*mA*mU*mG*mG*mU*mG*mC*mG*mG*mC*mA*mC*mG*mG*mG 3′ (SEQ ID NO: 160), mouse mismatched control 1 (mouse MM-1): 5′ mC*mU*mU*mG*mC*mU*mG*mU*mG*mG*mG*mA*mC*mC*mG*mG 3′ (SEQ ID NO: 161), mouse uORF-2 ASO (mouse ASO2): 5′ mC*mA*mU*mG*mG*mC*mC*mC*mC*mG*mG*mU*mU*mC*mC*mC3′ (SEQ ID NO: 162), and mouse mismatched control 2 (mouse MM-2): 5′ mC*mU*mU*mG*mC*mC*mC*mG*mC*mG*mC*mU*mU*mA*mC*mC3′ (SEQ ID NO: 163). In the ASOs of this paragraph, “m” indicates that these ASOs have 2′-O-methyl (2′-OMe) modification to the sugar group, and “*” indicates that the linkage is a phosphorothioate (PS) linkage.

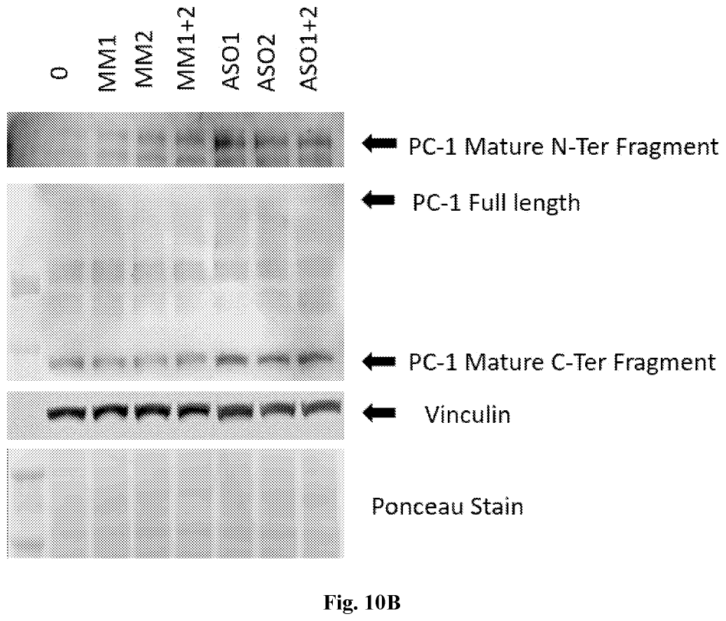

The two mouse ASOs and two control ASOs were tested in a mouse cell line (which contains an HA epitope tag on the C-terminus of Pkd1 to allow for assessment of the C-terminal fragment). Referring to FIGS. 10A and 10B, the treatment of the mouse cell line with 20 nM mouse ASO1, ASO2 or combinations of the two ASOs significantly increased the level of mature PC1 protein in the cell line.

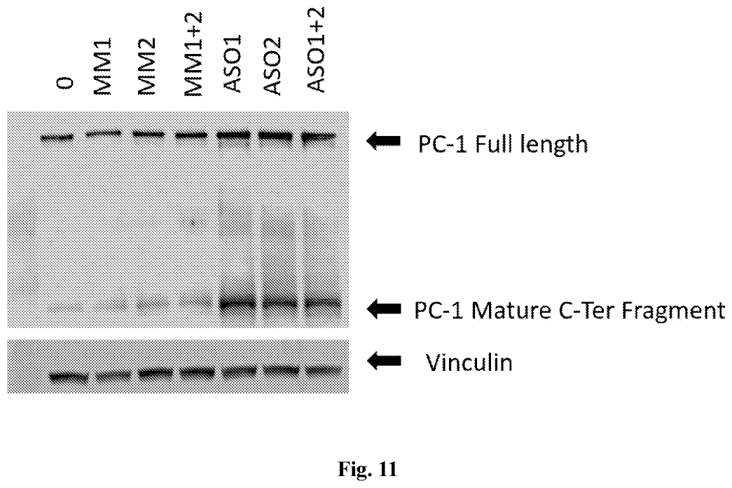

The ASOs were then tested in another mouse cell line that contains a missense mutation R2216W in the PC1 protein, as well as a V5 epitope tag on the C-terminus of the same protein for detection. Mouse R2216W mutation corresponds to the R2220W mutation in the human PC1 protein, which is found in some ADPKD patients, and has been shown to affect the cleavage and maturation of the protein (Vujic et al. J Am Soc Nephrol. 2010 July; 21 (7): 1097-102, and Krappitz et al JASN 2023) Referring to FIG. 11, even for the mutant PC1 R2216W protein, which has a tendency to resist cleavage and maturation, the treatment with the ASOs resulted in significantly increased levels of the mature PC1 protein (cleaved protein).

### Example 2: (Hypothetical) ASOs Suppressing uORF3 and/or uORF4 Increase PC1 Protein Levels

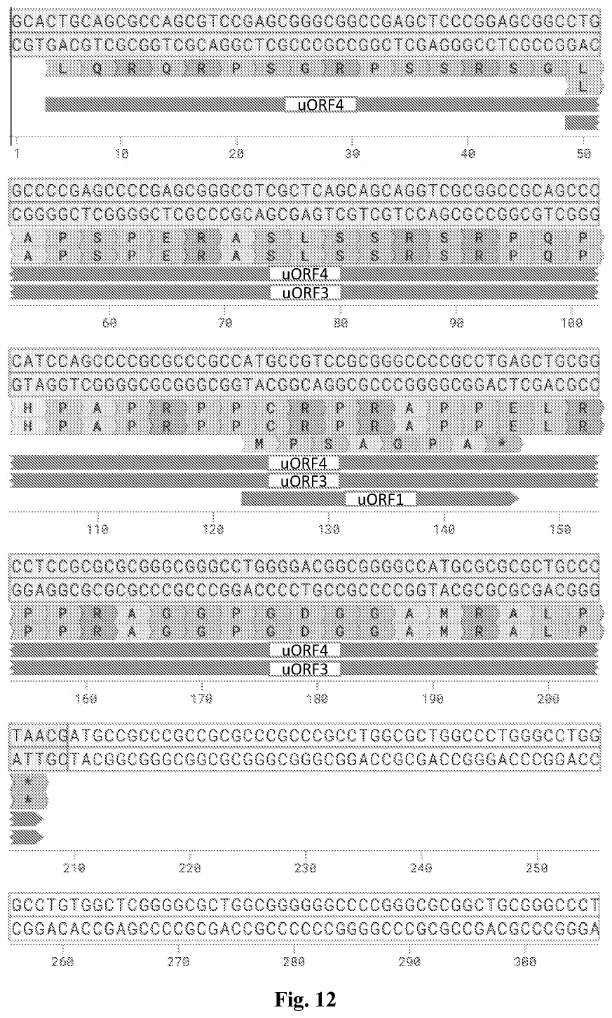

In addition to the uORF1 and uORF2 above, further analysis of the human PKD1 mRNA 5′-UTR sequence revealed two additional potential uORFs: uORF3 and uORF4 for which there was evidence of translation in at least one publicly available ribosome profiling experiment. (FIG. 12).

ASOs are designed to suppress uORF3 and uORF4. Specifically, the ASOs are designed bind to the human PKD1 mRNA molecule at locations either close to or including the initiation codons of uORF3 or uORF4 (both uORF3 and uORF4 have “CUG” as the start codon), and hinder the translation of either or both of these two uORFs. Tests of the ASOs (such as in human cell lines) show that these ASOs are able to increase the expression of the PC1 protein, as well as the mature form thereof, similar to ASO1/2 that suppress uORF1/2.

### Example 3: (Hypothetical) ASOs Inhibiting uORFs in Pkd1 5′UTR Decreases Cystic Phenotype in ADPKD Mouse Model

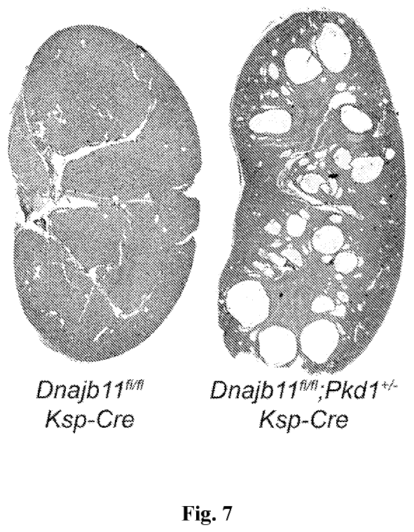

Referring to FIG. 7, knocking out Dnajb11 and one copy of Pkd1 during embryogenesis (Ksp-Cre is active in distal nephron from mid-embryogenesis) results in strong cystic phenotype in mice.

Using this mouse disease model, it is found that the administration of mouse uORF ASOs significantly increases PC1 protein expression from the single remaining allele, where a two-fold increase is expected to abolish the cystic phenotype in the kidney of the mice.

### Example 4: (Hypothetical) Evaluation of CTG Initiated uORF by Western Blot

Retinal Pigment Epithelium (RPE) cells are cultured, and either vehicle, ASO3 (targeting uORF3), negative control mismatched ASO3 (“ASOMM3”, an altered sequence that doesn't actually target the uORF3), ASO1 (positive controls), or ASO1+ASO3 is applied for 24 or 96 hours, protein lysate is made from the cells and is evaluated for Polycystin-1 (PC1) expression by western blot.

The experiment is repeated to compare the normalized luciferase expression from a wild-type PKD1 5′UTR-Renilla luciferase construct treated with vehicle, ASO3, ASOMM3, ASO1, or ASO1+ASO3.

The effect on PC1 expression of blocking the uORF3 will be similar to the effects of blocking the other uORFs described herein.

A further experiment will modify the sequence of the PKD1-5′UTR in our psiCHECK2 luciferase expression plasmid that contains the human PKD1 5′UTR to contain the genetic sequence modification ΔuORF3, or ΔuORF4 combinations of these and ΔuORF1 or ΔuORF2, analogous to that illustrated in FIG. 4B. The genetic sequence modification will change the CTG initiation codon of uORF3 or uORF4 to TTG to prevent translation initiation. Renilla: Firefly luciferase expression from the genetically-modified construct compared with that of the wild-type construct (baseline) and that of the delta-uORF1 construct (positive control based on illustrated results).

### ENUMERATED EMBODIMENTS

In some embodiments, the instant specification is directed to the following non-limiting embodiments:

Embodiment 1: A method of treating, ameliorating and/or preventing an autosomal dominant polycystic kidney disease (ADPKD) or a polycystic liver disease (PCLD) in a subject in need thereof, comprising:
administering to the subject an effective amount of a compound that suppresses the translation of a first upstream open reading frame (uORF), a second uORF, a third uORF, and/or a fourth uORF of the PKD1 gene.

Embodiment 2: The method of embodiment 1, wherein the method is a method of treating, ameliorating and/or preventing the ADPKD in the subject, and wherein the ADPKD is caused by or involves a mutation in the PKD1 gene in the subject.

Embodiment 3: The method of embodiment 1, wherein the method is a method of treating, ameliorating and/or preventing the PCLD in the subject, and wherein the PCLD is caused by or involves a germline mutation of the PKD1 gene, the PKD2 gene, the PRKCSH gene, the SEC63 gene, the GANAB gene, the ALG8 gene, the ALG9 gene, the SEC61B gene, or the DNAJB11 gene in the subject.

Embodiment 4: The method of embodiments 1-3, wherein the compound comprises:
CRISPR components that disrupt the genomic DNA sequence that encoding the first uORF, the second uORF, the third uORF, and/or the fourth uORF, or an expression vector expressing the CRISPR components; or
an antisense oligonucleotide (ASO) that blocks the translation of the first uORF, the second uORF, the third uORF, and/or the fourth uORF, or an expression vector expressing the ASO.

Embodiment 5: The method of embodiment 4, wherein the compound comprises the CRISPR components or the expression vector expressing the CRISPR components, and wherein the CRISPR components disrupt the initiation codon of the first uORF, the second uORF, the third uORF, and/or the fourth uORF.

Embodiment 6: The method of embodiment 4, wherein the compound comprises the ASO or the expression vector expressing the ASO, and wherein the portion of the PKD1 mRNA complementary to the ASO extends to 5 nucleotides away or less from the initiation codon of the first uORF, the second uORF, the third uORF, or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF, or the fourth uORF.

Embodiment 7: The method of embodiments 4 or 6, wherein a length of the ASO is 10 nucleotides or longer, such as 11 nucleotides or longer, 12 nucleotides or longer, 13 nucleotides or longer, 14 nucleotides or longer or 15 nucleotides or longer.

Embodiment 8: The method of embodiments 4 and 6-7, wherein a length of the ASO is 30 nucleotides or shorter, such as 29 nucleotides or shorter, 28 nucleotides or shorter, 27 nucleotides or shorter, 26 nucleotides or shorter or 25 nucleotides or shorter.

Embodiment 9: The method of embodiments 4 and 6-8, wherein at least one of the following applies:
(a) the ASO is fully complementary with one sequence set forth in SEQ ID NOs: 14-61,
(b) the ASO is fully complementary with one sequence set forth in SEQ ID NOs: 62-109,
(c) the ASO is fully complementary with one sequence set forth in SEQ ID NOs: 110-157,
(d) the ASO comprises the nucleotide sequence CAUGGCGGGCGCGGGG (SEQ ID NO:158), the nucleotide sequence CAUGGCCCCGCCGUCC (SEQ ID NO: 159).

Embodiment 10: The method of embodiments 4 and 6-9, wherein the ASO comprises a modified nucleobase, a modified sugar group, or a modified linkage.

Embodiment 11: The method of embodiment 10, wherein at least one of the following applies:
(a) the ASO comprises the modified sugar group, and the modified sugar group comprise a 2′-O-methylation modified sugar group, such as a 2′-O-methylation modified ribose group,
(b) the ASO comprises the modified linkage, and the modified linkage comprise a phosphorothioate (PS) linkage.

Embodiment 12: The method of embodiments 1-11, wherein the subject is a mammal, such as a human.

Embodiment 13: The method of embodiments 1-12, wherein the compound comprises the ASO or the expression vector expressing the ASO, and wherein a concentration of the ASO in kidney or lung of the subject ranges from about 1 nm to about 100 nm.

Embodiment 14: A method of increasing PKD1 expression in a cell, comprising:
contacting with the cell an effective amount of a compound that suppresses the translation of the first upstream open reading frame (uORF), the second uORF, the third uORF, and/or the fourth uORF of the PKD1 gene.

Embodiment 15: The method of embodiment 14, wherein the cell has a mutation in the PKD1 gene, the PKD2 gene, the PRKCSH gene, the SEC63 gene, the GANAB gene, the ALG8 gene, the ALG9 gene, the SEC61B gene, or the DNAJB11 gene.

Embodiment 16: The method of any one of embodiments 14-15, wherein the cell is in a tissue or a subject.

Embodiment 17: The method of any one of embodiments 14-16, wherein the cell is a kidney cell in a subject diagnosed with autosomal dominant polycystic kidney disease (ADPKD) or a liver cell in a subject diagnosed with polycystic liver disease (PCLD).

Embodiment 18: The method of any one of embodiments 14-17, wherein the compound comprises:
CRISPR components that disrupt the genomic DNA sequence that encoding the first uORF, the second uORF, the third uORF and/or the fourth uORF, or an expression vector expressing the CRISPR components; or
an antisense oligonucleotide (ASO) that blocks the first uORF, the second uORF, the third uORF and/or the fourth uORF, or an expression vector expressing the ASO.

Embodiment 19: The method of embodiment 18, wherein the compound comprises the CRISPR components or the expression vector expressing the CRISPR components, and wherein the CRISPR components disrupt the initiation codon of the first uORF, the second uORF, the third uORF and/or the fourth uORF.

Embodiment 20: The method of embodiment 18, wherein the compound comprises the ASO or the expression vector expressing the ASO, and wherein the portion of the PKD1 mRNA complementary to the ASO extends to 5 nucleotides away or less from the initiation codon of the first uORF, the second uORF, the third uORF or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF or the fourth uORF.

Embodiment 21: The method of embodiment 18 or embodiment 20, wherein a length of the ASO is 10 nucleotides or longer, such as 11 nucleotides or longer, 12 nucleotides or longer, 13 nucleotides or longer, 14 nucleotides or longer or 15 nucleotides or longer.

Embodiment 22: The method of any one of embodiments 18 and 20-21, wherein a length of the ASO is 30 nucleotides or shorter, such as 29 nucleotides or shorter, 28 nucleotides or shorter, 27 nucleotides or shorter, 26 nucleotides or shorter or 25 nucleotides or shorter.

Embodiment 23: The method of any one of embodiments 18 and 20-22, wherein at least one of the following applies:
(a) the ASO is fully complementary with one sequence set forth in SEQ ID NOs: 14-61,
(b) the ASO is fully complementary with one sequence set forth in SEQ ID NOs: 62-109,
(c) the ASO is fully complementary with one sequence set forth in SEQ ID NOs: 110-157,
(d) the ASO comprises the nucleotide sequence CAUGGCGGGCGCGGGG (SEQ ID NO:158), the nucleotide sequence CAUGGCCCCGCCGUCC (SEQ ID NO:159).

Embodiment 24: The method of any one of embodiments 18 and 20-23, wherein the ASO comprises a modified nucleobase, a modified sugar group or a modified linkage.

Embodiment 25: The method of embodiment 24, wherein at least one of the following applies:
(a) the ASO comprises the modified sugar, and the modified sugar group comprise a 2′-O-methylation modified sugar group, such as a 2′-O-methylation modified ribose group,
(b) the ASO comprises the modified linkage, and the modified linkage comprise a phosphorothioate (PS) linkage.

Embodiment 26: The method of any one of embodiments 14-24, wherein the compound comprises the ASO or the expression vector expressing the ASO, and wherein a concentration of the ASO contacted with the cell ranges from about 1 nm to about 100 nm.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating and/or preventing an autosomal dominant polycystic kidney disease (ADPKD) or a polycystic liver disease (PCLD) in a subject in need thereof, comprising:
administering to the subject an effective amount of a compound that suppresses the translation of a first upstream open reading frame (uORF), a second uORF, a third uORF, and/or a fourth uORF of the PKD1 gene.

2. The method of claim 1, wherein the method is a method of treating, ameliorating and/or preventing the ADPKD in the subject, and wherein the ADPKD is caused by or involves a mutation in the PKD1 gene in the subject.

3. The method of claim 1, wherein the method is a method of treating, ameliorating and/or preventing the PCLD in the subject, and wherein the PCLD is caused by or involves a germline mutation of the PKD1 gene, the PKD2 gene, the PRKCSH gene, the SEC63 gene, the GANAB gene, the ALG8 gene, the ALG9 gene, the SEC61B gene, or the DNAJB11 gene in the subject.

4. The method of claim 1, wherein the compound comprises:
CRISPR components that disrupt the genomic DNA sequence that encoding the first uORF, the second uORF, the third uORF, and/or the fourth uORF, or an expression vector expressing the CRISPR components; or
an antisense oligonucleotide (ASO) that blocks the translation of the first uORF, the second uORF, the third uORF, and/or the fourth uORF, or an expression vector expressing the ASO.

5. The method of claim 4, wherein the compound comprises the CRISPR components or the expression vector expressing the CRISPR components, and wherein the CRISPR components disrupt the initiation codon of the first uORF, the second uORF, the third uORF, and/or the fourth uORF.

6. The method of claim 4, wherein the compound comprises the ASO or the expression vector expressing the ASO, and wherein the portion of the PKD1 mRNA complementary to the ASO extends to 5 nucleotides away or less from the initiation codon of the first uORF, the second uORF, the third uORF, or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF, or the fourth uORF.

7. The method of claim 4, wherein a length of the ASO is 10 nucleotides or longer, such as 11 nucleotides or longer, 12 nucleotides or longer, 13 nucleotides or longer, 14 nucleotides or longer or 15 nucleotides or longer.

8. The method of claim 4, wherein a length of the ASO is 30 nucleotides or shorter, such as 29 nucleotides or shorter, 28 nucleotides or shorter, 27 nucleotides or shorter, 26 nucleotides or shorter or 25 nucleotides or shorter.

9. The method of claim 4, wherein at least one of the following applies:
(a) the ASO is fully complementary with one sequence set forth in SEQ ID NOs: 14-61,
(b) the ASO is fully complementary with one sequence set forth in SEQ ID NOs: 62-109,
(c) the ASO is fully complementary with one sequence set forth in SEQ ID NOs: 110-157,
(d) the ASO comprises the nucleotide sequence CAUGGCGGGCGCGGGG (SEQ ID NO: 158), the nucleotide sequence CAUGGCCCCGCCGUCC (SEQ ID NO:159).

10. The method of claim 4, wherein the ASO comprises a modified nucleobase, a modified sugar group, or a modified linkage.

11. The method of claim 10, wherein at least one of the following applies:
(a) the ASO comprises the modified sugar group, and the modified sugar group comprise a 2′-O-methylation modified sugar group, such as a 2′-O-methylation modified ribose group,
(b) the ASO comprises the modified linkage, and the modified linkage comprise a phosphorothioate (PS) linkage.

12. The method of any one of claim 1, wherein the subject is a mammal, such as a human.

13. The method of claim 1, wherein the compound comprises the ASO or the expression vector expressing the ASO, and wherein a concentration of the ASO in kidney or lung of the subject ranges from about 1 nm to about 100 nm.

14. A method of increasing PKD1 expression in a cell, comprising:
contacting with the cell an effective amount of a compound that suppresses the translation of the first upstream open reading frame (uORF), the second uORF, the third uORF, and/or the fourth uORF of the PKD1 gene.

15. The method of claim 14, wherein the cell has a mutation in the PKD1 gene, the PKD2 gene, the PRKCSH gene, the SEC63 gene, the GANAB gene, the ALG8 gene, the ALG9 gene, the SEC61B gene, or the DNAJB11 gene.

16. The method of claim 14, wherein the cell is in a tissue or a subject.

17. The method of claim 14, wherein the cell is a kidney cell in a subject diagnosed with autosomal dominant polycystic kidney disease (ADPKD) or a liver cell in a subject diagnosed with polycystic liver disease (PCLD).

18. The method of claim 14, wherein the compound comprises:
CRISPR components that disrupt the genomic DNA sequence that encoding the first uORF, the second uORF, the third uORF and/or the fourth uORF, or an expression vector expressing the CRISPR components; or
an antisense oligonucleotide (ASO) that blocks the first uORF, the second uORF, the third uORF and/or the fourth uORF, or an expression vector expressing the ASO.

19. The method of claim 18, wherein the compound comprises the CRISPR components or the expression vector expressing the CRISPR components, and wherein the CRISPR components disrupt the initiation codon of the first uORF, the second uORF, the third uORF and/or the fourth uORF.

20. The method of claim 18, wherein the compound comprises the ASO or the expression vector expressing the ASO, and wherein the portion of the PKD1 mRNA complementary to the ASO extends to 5 nucleotides away or less from the initiation codon of the first uORF, the second uORF, the third uORF or the fourth uORF, such as extend to 4 nucleotides away or less, extend to 3 nucleotides away or less, extend to 2 nucleotides away or less, extend to 1 nucleotide away or less, reaches the boundary of the initiation codon, reaches 1 nucleotide or more of the initiation codon, reaches 2 nucleotides or more of the initiation codon, or reaches the entirety of the initiation codon of the first uORF, the second uORF, the third uORF or the fourth uORF.

21. The method of claim 18, wherein a length of the ASO is 10 nucleotides or longer, such as 11 nucleotides or longer, 12 nucleotides or longer, 13 nucleotides or longer, 14 nucleotides or longer or 15 nucleotides or longer.

22. The method of claim 18, wherein a length of the ASO is 30 nucleotides or shorter, such as 29 nucleotides or shorter, 28 nucleotides or shorter, 27 nucleotides or shorter, 26 nucleotides or shorter or 25 nucleotides or shorter.

23. The method of claim 18, wherein at least one of the following applies:
(a) the ASO is fully complementary with one sequence set forth in SEQ ID NOs: 14-61,
(b) the ASO is fully complementary with one sequence set forth in SEQ ID NOs: 62-109,
(c) the ASO is fully complementary with one sequence set forth in SEQ ID NOs: 110-157,
(d) the ASO comprises the nucleotide sequence CAUGGCGGGCGCGGGG (SEQ ID NO: 158), the nucleotide sequence CAUGGCCCCGCCGUCC (SEQ ID NO:159).

24. The method of claim 18, wherein the ASO comprises a modified nucleobase, a modified sugar group or a modified linkage.

25. The method of claim 24, wherein at least one of the following applies:
(a) the ASO comprises the modified sugar, and the modified sugar group comprise a 2′-O-methylation modified sugar group, such as a 2′-O-methylation modified ribose group,
(b) the ASO comprises the modified linkage, and the modified linkage comprise a phosphorothioate (PS) linkage.

26. The method of claim 14, wherein the compound comprises the ASO or the expression vector expressing the ASO, and wherein a concentration of the ASO contacted with the cell ranges from about 1 nm to about 100 nm.
